(19) United States
(12) Patent Application Publication
(10) Pub. No.: US 2023/0166092 A1
(43) Pub. Date: Jun. 1, 2023

(54) ULTRASOUND MEDIATED NON-INVASIVE DRUG DELIVERY POROUS CARRIERS

(71) Applicant: MuPharma Pty Ltd, Malvern, Victoria (AU)

(72) Inventors: Harry Unger, Toorak (AU); Mark Unger, Malvern (AU); Nicolas Hans Voelcker, Blackwood (AU)

(21) Appl. No.: 17/927,617

(22) PCT Filed: May 28, 2021
(86) PCT No.: PCT/AU2021/050530

§ 371 Date: Nov. 23, 2022

(30) Foreign Application Priority Data
May 28, 2020 (AU) 2020901747

(51) Int. Cl. A61M 37/00

(52) U.S. Cl. CPC A61M 37/0092; A61M 2205/0294

(57) ABSTRACT

The present invention relates to agent delivery systems for application to biological tissues. More specifically, the present invention relates to devices and methods for the non-invasive delivery of agents (e.g. pharmaceuticals and the like) into and across biological tissues using ultrasound and nanoporous carriers.

### CROSS-REFERENCE TO RELATED APPLICATION

The present application claims priority from Australian provisional patent application number 2020901747, the entire content of which is incorporated herein by cross-reference.

### TECHNICAL FIELD

The present invention relates to agent delivery systems for application to biological tissues. More specifically, the present invention relates to devices and methods for the non-invasive delivery of agents (e.g. pharmaceuticals and the like) into and across biological tissues using ultrasound and nanoporous carriers.

### BACKGROUND

Upon administration of given pharmaceutical drug, the active ingredient responsible for the desired therapeutic effect generally needs to move across biological barrier/s to reach target tissue or cells. In many cases, only a minor portion of the drug administered reaches the therapeutic target, while the remainder distributes in healthy tissues. This compromises the therapy and often culminates in undesired adverse effects.

Many existing methods of delivering drugs to patients utilise oral administration or injection. However, it is well recognised that these methods have reduced efficacy for some therapies. For example, oral delivery is a commonly regarded as a convenient and preferred route for drug delivery. Numerous existing drugs and newly-identified chemical entities suffer from poor aqueous solubility, inadequate dissolution throughout the gastrointestinal tract, and consequently reduced bioavailability when they are administered orally. Oral drug delivery is also limited by an inability to deliver larger therapeutic molecules (e.g. proteins). Conversely, delivery by injection allows the delivery of macromolecules, but is hampered by its invasive nature and inappropriate use (e.g. unsafe practices, incorrect technique etc. Collectively, tablets and injections are unable to adequately meet many therapeutic needs including, for example, targeting, tight dosage control at the level of the target tissue or cells, broad applicability to macromolecules, and on-demand activation. While not all pharmaceuticals require these abilities for effective use there are many that do.

There is a need for new drug delivery systems that can effectively improve existing therapies by providing benefits such as, for example, any one or more of increased drug carrier loading accuracy and predictability, controlled release of drugs for dosage accuracy and/or elimination of drug waste and/or off-target tissue drug delivery, enhanced ultrasound transmission to cells/tissue and/or uptake, and/or reduced damage (i.e. invasiveness) to tissue in drug delivery.

### SUMMARY OF THE INVENTION

The present invention addresses at least one problem with existing agent delivery systems by providing devices and methods for the non-invasive delivery of agents into biological tissues using a combination of ultrasound and nanoporous carriers.

While not limiting and not intending to be bound by theory, it is proposed that the use of agent carriers having a high density of nanoscale features (e.g. nanoscale channels and/or nanoscale pores and/or nanoscale porous networks) increases the surface area (which can impart ultrasound to tissue) to volume (agent containing capacity) ratio of such agent carriers. This reduces the weight of an agent carrier and may in turn reduce the power needed to achieve the desired sonophoretic (i.e. ultrasound-mediated) effect for delivery of the agent through tissue, and may thus reduce the heat and stress applied to the tissue. Additionally, the high surface area of nanoscale porous features increases the radiating contact area of ultrasound again reducing the amount of power needed to facilitate delivery via sonophoresis and making the process less disruptive to the treated tissue. In some instances, the speed and concentration of agents delivered to tissues can be considerably increased using the devices and methods of the present invention. Moreover, preferred porous carriers of the present invention (e.g. nanoporous silicon) can be made according to a porosity density which confers a desired surface area to volume ratio. Further, the pore density and/or pore size i.e., the average pore diameter, may be tuned in order to suit the molecular size of a relevant agent.

Additionally or alternatively, using the porous devices of the present invention alters the wettability of the agent carrier's surface and as such can serve to eliminate or reduce the meniscus on the top surface of a tip of the device when loaded with an agent-containing liquid. Such a property is beneficial from the therapeutic perspective as a meniscus at the tip may interfere with dosages, experimental accuracy and can lead to wastage of the agent because when such meniscus is applied to tissue, it may be extruded away from the tip, outside of the region of ultrasonic excitation. Reduction or elimination of this meniscus formation overcomes these issues and greatly assists in the precise volumetric loading the substrate which is not trivial at all when working with small volumes of 5-50 μl, for example.

The present invention provides devices and methods utilising ultrasound as a means of facilitating the non-invasive delivery of agents from porous nanoscale structures (e.g. nanoscale channels) into or through a target tissue. Existing devices typically utilise iontophoresis to permeate tissue surfaces which have shown limited efficacy and have demonstrated limited market penetration.

The devices and methods of the present invention may rely entirely on ultrasound to deliver agents into or through the target tissue, and have no requirement to utilise electromotive forces or differences in electric potential to deliver the agent (e.g. iontophoresis (ionisation), iontophoresis, electrophoresis, nanoscale electrophoresis, electroosmosis, cataphoresis, electroendosmosis, electrorepulsion and the like).

The devices and methods can be used in methods for the delivery of agents into or through target tissues for the prevention and/or treatment of conditions/diseases and/or for any other purpose. For example, as described herein the devices and methods may be used for the delivery of agents into or through various tissues including the eye, skin and mucosal surfaces. In some embodiments, the devices may be used to deliver agents into or through mucosal surfaces and thereby induce an immune response. The immune response induced in these embodiments of the invention can be a mucosal immune response, a systemic immune response, or both. Preferably, at least a mucosal immune response is induced, and optionally a systemic immune response is also induced. It is considered that by selectively configuring the operational parameters of the agent applicator presently described, the amount of agent delivered to a selected depth or one or more layers of a tissue may be controlled. For example, in some embodiments of the present and previous aspects of the invention, there is provided delivery of the agent to induce at least a mucosal immune response by controlling the delivery of the agent such that majority of the agent is delivered into the epithelial and sub-epithelial layer of the mucous membrane.

Accordingly, in some embodiments of the invention, delivery of the agent induces at least a mucosal immune response. The agent may be applied using the operational parameters described herein, and a sufficient dose of agent may remain resident in the mucous membrane, at least temporarily, in order to induce an immune response in the mucous membrane. More specifically, a sufficient dose of agent may remain resident at least temporarily in one or more of the epithelial or sub-epithelial layers of the mucous membrane.

The agent carriers and methods of the present invention aim, among other things, to control the depth of delivery of a given agent (e.g. a drug) into tissue. Depending on the desired therapeutic effect of a particular drug, it may be beneficial that the amount of systemic delivery of a drug is limited. Some examples where limiting the range of depth of delivery of a drug into tissue can be useful include the superficial mucosal delivery of vaccines to induce strong mucosal immunity and, in the example of the eye, the delivery of riboflavin-5-phosphate sodium salt to the superficial half thickness of the cornea to enable treatment of keratoconus (conical cornea) by corneal collagen cross-linking using ultraviolet light.

Embodiments of the present invention, by utilising a non-invasive drug delivery system that can control the depth of penetration of drugs into tissues, provide a novel approach and solution to a range of unmet medical needs.

In one embodiment, the present invention provides a device, comprising:

an agent carrier comprising an agent transfer surface for delivery of an agent into a tissue, wherein the agent carrier comprises or is acoustically couplable to a piezoelectric substrate; an electrode electrically couplable to the piezoelectric substrate; and

a controller electrically couplable to the electrode and configured to apply an electrical signal to the electrode to propagate an acoustic wave on and/or in the piezoelectric substrate which is capable of delivering the agent from the agent carrier into the tissue.

The agent carrier may be provided in the form of a consumable applicator tip adapted for one-time use.

The agent carrier may comprise a plurality of nanoscale channels extending partially or wholly through the agent carrier to the agent transfer surface enabling retention of the agent and/or transportation of the agent to the tissue. The porosity of the plurality of nanoscale channels may account for up to 60%, 70%, 80% or 85% of the total agent carrier volume. The plurality of nanoscale channels and/or pores at the tissue contacting surface of the agent carrier which are in fluid communication with the nanoscale channels may range in maximum width (e.g. diameter) from more than 1 nm to less than 1 micrometer (μm) such as, for example, between 1 nm and 999 nm, between 5 nm and 999 nm, between 10 nm and 999 nm, between 50 nm and 999 nm, between 1 nm and 50 nm, between 5 nm and 500 nm, between 10 nm and 500 nm, between 50 nm and 500 nm, between 1 nm and 55 nm, from between 1 nm and 50 nm, from between 1 nm and 45 nm, from between 1 nm and 40 nm, from between 1 nm and 35 nm, from between 5 nm and 55 nm, from between 5 nm and 50 nm, from between 5 nm and 45 nm, from between 5 nm and 40 nm, from between 5 nm and 35 nm, from between 10 nm and 55 nm, from between 10 nm and 50 nm, from between 10 nm and 45 nm, from between 10 nm and 40 nm, and from between 10 nm and 35 nm. At a given cross section of the agent carrier, the plurality of nanoscale channels may be provided in an amount of at least: 5000, 6000, 7000, 8000, 9000, 10000, 11000, 12000, 13000, 14000, 15000, 20000, 25000, 50000, 75000 or 100000 channels/cm² of the agent carrier body,

In some embodiments of the present invention, the device uses ultrasonic power for the delivery of agents into tissue in the range 0.05 to 5.25 Wcm⁻². This may be appropriate for applications involving delivery of agents to mucous membranes, eyes and other delicate tissues. Higher intensity ultrasound may be needed in some applications for less-delicate tissues including skin, nails and tooth enamel. In these cases, it may be beneficial to pulse or modulate the duty cycle of the ultrasonic energy to prevent tissue damage, (e.g. from thermal effects) and/or to prevent damage to the agent. As will be appreciated by persons skilled in the art, the ultrasound may be applied over one or more frequency bands or over a frequency spectrum having several bands. In some embodiments, the band(s) may correspond to a resonant frequency of the agent applicator device including the agent carrier body, and optionally one or more harmonics of the resonant frequency.

In some embodiments of the present invention, ultrasound transmitted by the device is of a frequency of between 20 kHz to 100 kHz, for example between 20 kHz and 40 kHz. Ultrasound signals of this frequency when provided with sufficient intensity can (among other phenomena) cause micro cavitation events in tissues, which enhances their permeability. This may be appropriate for applications involving delivery of agents to mucous membranes, eyes and other delicate tissues. However, in other embodiments the device may have a resonant frequency lower than this, and the devices and methods describe herein may be operated with a primary resonant frequency at the tip of the agent carrier body of around 10 kHz. Devices suitable for use in the methods of the present invention may be operated at primary frequencies in any one or more of the following frequency bands, a band centred at or about 10 kHz; 20 kHz, 22 kHz, 27 kHz, 28 kHz, 28.19 kHz, and 38 kHz and/or frequency bands of 20-25 kHz, 25-30 kHz, 38-40 kHz, 40-45 kHz, 40 to 60 kHz. These primary frequencies may produce harmonic frequencies including in the bands of 40-80 kHz and 140-160 kHz which also may assist in causing micro cavitation events in tissues, that enhances their permeability.

The piezoelectric substrate of the device may comprise a single crystal piezoelectric material, a thin-film piezoelectric material, or a combination thereof. The piezoelectric substrate may comprise any one or more of lithium niobate, tourmaline, single-crystal quartz, and/or lead zirconate titanate.

The electrical signal applied by the controller may generate a primary acoustic excitation frequency on and/or in the piezoelectric substrate in a range of 1 MHz to 10 GHz. The primary acoustic excitation frequency may correspond to the resonant frequency of the piezoelectric substrate.

The electrical signal applied by the controller may generate a primary acoustic excitation frequency on and/or in the piezoelectric substrate in a range of 1 MHz to 100 GHz of any wave type. For example, the primary acoustic excitation frequency may be more than 10⁶ Hz, more than 10⁷ Hz, more than 10⁸ Hz, more than 10⁹ Hz, more than 10¹⁰ Hz, or more than 10¹¹ Hz. The primary acoustic excitation frequency may be, for example, between 10⁶ Hz and 10⁷ Hz, between 10⁶ Hz and 10⁸ Hz, between 10⁶ Hz and 10⁹ Hz, between 10⁶ Hz and 10¹⁰ Hz, between 10⁷ Hz and 10⁸ Hz, between 10⁷ Hz and 10⁹ Hz, between 10⁷ Hz and 10¹⁰ Hz, between 10⁸ Hz and 10⁹ Hz, between 10⁸ Hz and 10¹⁰ Hz, or between 10⁹ Hz and 10¹⁰ Hz. The primary acoustic excitation frequency may correspond to the resonant frequency of the piezoelectric substrate and/or the spatial arrangement of excitation transducers electrodes.

The device (e.g. the device controller) may further comprise an acoustic generator capable of generating one or more secondary acoustic excitation frequencies of any wave type (including square, sine sawtooth) or combination thereof capable of modulating the primary acoustic excitation on and/or in the piezoelectric substrate. The secondary acoustic excitation frequency may be less than or equal to the primary acoustic excitation frequency. For example, secondary acoustic excitation frequency acoustic excitation frequency may be 1 Hz to 100 kHz, 1 Hz, less than 10 Hz, less than 10² Hz, less than 10³ Hz, less than 10⁴ Hz, less than 10⁵ Hz, less than 10⁶ Hz, less than 10⁷ Hz, less than 10⁸ Hz, less than 10⁹ Hz, less than 10¹⁰ Hz, or less than 10¹¹ Hz. The supplementary, alternative or otherwise additional acoustic frequency may, for example, be between 1 Hz and 10 Hz, between 1 Hz and 10² Hz, between 1 Hz and 10³ Hz, between 1 Hz and 10⁴ Hz, between 1 Hz and 10⁵ Hz, between 1 Hz and 10⁶ Hz, between 10 Hz and 10² Hz, between 10 Hz and 10³ Hz, between 10 Hz and 10⁴ Hz, between 10 Hz and 10⁵ Hz, between 10 Hz and 10⁶ Hz, between 10³ Hz and 10⁴ Hz, between 10³ Hz and 10⁵ Hz, between 10³ Hz and 10⁶ Hz, between 10⁴ Hz and 10⁵ Hz, between 10⁴ Hz and 10⁶ Hz, between 10⁵ Hz and 10⁶ Hz, between 10⁶ Hz and 10⁷ Hz, between 10⁶ Hz and 10⁸ Hz, between 10⁶ Hz and 10⁹ Hz, between 10⁶ Hz and 10¹⁰ Hz, between 10⁷ Hz and 10⁸ Hz, between 10⁷ Hz and 10⁹ Hz, between 10⁷ Hz and 10¹⁰ Hz, between 10⁸ Hz and 10⁹ Hz, between 10⁸ Hz and 10¹⁰ Hz, or between 10⁹ Hz and 10¹⁰ Hz. The wave type, frequency level, number and duration of additional frequencies may vary throughout the duration in which the primary acoustic excitation signal is applied to tissue. When applied to tissue the acoustic frequency signal may make it more permeable.

The acoustic wave propagated on and/or in the piezoelectric substrate may not be a bulk (lamb) wave. The acoustic wave may be a surface acoustic wave. The acoustic wave may be a Rayleigh surface acoustic wave.

The device may be incapable of utilising electromotive force to transport a charged agent into and/or through a tissue in contact with the agent transfer surface of the device.

Additionally or alternatively, the device may be incapable of generating or maintaining a difference in electric potential between the agent transfer surface of the device and the tissue surface in contact with it to consequently induce transport of the agent from the device into the tissue.

Additionally or alternatively, the device may be incapable of:

(i) utilising repulsive electromotive force to transport a charged agent into and/or through the tissue in contact with the agent transfer surface; and/or

(ii) permeating the tissue by any of iontophoresis (ionization), iontophoresis, electrophoresis, microelectrophoresis, electroosmosis, cataphoresis, electroendosmosis, and electrorepulsion.

The device may further comprise the agent.

The device may include the following features:
the agent carrier may comprise the piezoelectric substrate,
the piezoelectric substrate may comprise the agent transfer surface, and
the agent may be present on the agent transfer surface.

The device may include the following features:
the agent transfer surface may be functionalised (e.g. chemically), and/or
the agent may be lyophilised on the agent transfer surface,
to thereby retain the agent on the agent transfer surface.

The agent carrier of the device may comprise any one or more of: a plurality of nanoscale channels and a reservoir, or a combination thereof.

The agent carrier of the device may have volumetric retention capabilities and comprise any one or more of: a non-porous solid material which has been treated such that nanoscale channels and reservoirs, or a combination thereof are created. The fluid contained in porous agent carriers is in contact with itself so that there is a continuous fluid medium. The fluid contained in the non-porous nanoscale-machined material may be a continuous fluid medium.

The agent carrier of the device may comprise a network and/or multiplicity of plurality of nanoscale channels, extending at least partially or wholly through the agent carrier to the agent transfer surface enabling retention (e.g. volumetric retention) of the agent and/or transportation of the agent to the tissue.

The nanoscale channels of the device may be any one or more of cylindrical, cone or other shapes including random shapes. In some embodiments, some or all of the nanoscale channels of the device may directly interconnect.

The agent carrier of the device may comprise a stack of layers, and the stack of layers may comprise:

a first layer comprising the agent transfer surface; and

at least one other layer,

wherein holes formed in one layer of the plurality of layers are aligned with holes in an adjacent layer and in an arrangement facilitating a plurality of holes in a plurality of layers to cooperate to form the nanoscale channels.

The nanoscale channels of the device may extend from the interior of the agent carrier body and terminate as pores at the agent transfer surface.

The channels described above can in principle be formed on any surface including flat, concave or convex surfaces including on any nano-scale structures formed on such flat, concave or convex surfaces.

The agent carrier of the device may be formed from any one or more of a plastic or other type of polymer (including epoxy resin), a metal, silicon, porated silicon, germanium, and/or ceramic. The agent carrier may be formed from silicon.

The agent carrier of the device may be produced by three-dimensional (3D) printing of material, such as for example, polymeric material, metallic material, ceramic material and combinations thereof.

The agent carrier may be fabricated using any one or more of the following techniques; electrochemical dissolution, net shape manufacturing, near net shape manufacturing, additive manufacturing, nanofabrication, stereo-photolithography. The agent carrier may embody the different morphological aspects of a stochastic etch and/or electrochemical dissolution process. The agent carrier may also be designed and manufactured using pre-determined designs tailored to the end application such as, Computer Aided Design/Manufacture CAD/CAM techniques.

Given the nanoscale dimensions of the channels and pores of the agent carrier, agents suitable for delivery by the device typically have a maximum width of less than 1 μM such as, for example, less than: 900 nm, 800 nm, 700 nm, 600 nm, 500 nm, 400 nm, 300 nm, 200 nm, 100 nm, 50 nm, 40 nm, 30 nm, 20 nm, 10 nm, or 5 nm. The agent of the device may comprise a solid or a combination of a solid and a liquid. The agent may comprise a solid comprising powder, granules, or a combination thereof. The agent may be lyophilised. The agent may comprise a therapeutic agent, a prophylactic agent, a diagnostic agent, a cosmetic agent, or any combination thereof. The agent may be selected from the group consisting of: a protein, a peptide, a polypeptide, an antibody (including monoclonal and polyclonal antibodies), an immunogenic agent, a vaccine, a biomimetic, a biosimilar, a biomaterial, a macromolecule, a small molecule, a sugar, a nucleic acid, a drug, a nanoparticle, and any combination thereof.

In another embodiment, the present invention provides a method for delivering an agent to an internal layer within a target tissue, the method comprising:

contacting the target tissue with the agent transfer surface of a device of the present invention, and

applying an electrical signal to the electrode of the device to propagate acoustic waves on and/or in the piezoelectric substrate of the device, and thereby deliver the agent through the agent transfer surface to the internal layer of the target tissue. The target tissue may be intact tissue, and the agent transfer surface may be configured to inhibit or prevent mechanical penetration of a surface of the target tissue when in contact with it during standard use of the device.

“Intact tissue” as referred to herein will be understood to include undamaged tissue but also damaged tissue that retains sufficient integrity to support contact with the tissue contacting surface during standard use of the device.

As referred to herein, “standard use” of the device will be understood to require that tissue in contact with the device is not mechanically penetrated, pierced or destroyed by any part of the device itself, or acoustically pierced or destroyed by ultrasonic waves emanating from the device.

The target tissue may be skin. The target tissue may be mucosal tissue and the agent transfer surface may be configured to inhibit or prevent mechanical penetration of an intact epithelial layer of the mucosal tissue during standard use of the device. The target tissue may also be ocular tissue and the agent transfer surface may be configured to inhibit or prevent mechanical penetration of an intact conjunctiva or corneal epithelial layer (or both) during standard use of the device.

In another embodiment, the present invention provides a method for inducing mucosal immunity in a subject, the method comprising:

contacting a target mucosal tissue of the subject with the agent transfer surface of a device of the present invention, and

applying an electrical signal to the electrode of the device to propagate acoustic waves on and/or in the piezoelectric substrate of the device, and thereby deliver the agent through the agent transfer surface into the target mucosal tissue,

wherein delivery of the agent into the target mucosal tissue induces the mucosal immunity. The target mucosal tissue may be intact and the agent transfer surface may not penetrate an intact epithelial layer of the target mucosal tissue during standard use of the device.

In another embodiment, the present invention provides an agent for use in a method of preventing or treating a disease in a subject, wherein the agent is present in a device comprising:

a piezoelectric substrate;

an agent carrier comprising an agent transfer surface for delivery of an agent into a tissue, wherein the agent carrier comprises or is acoustically couplable to a piezoelectric substrate;

an electrode electrically couplable to the piezoelectric substrate; and

a controller electrically couplable to the electrode and configured to apply an electrical signal to the electrode,

wherein the method comprises using the controller to apply the electrical signal to the electrode of the device to propagate an acoustic wave on and/or in the piezoelectric substrate, and thereby deliver the agent through the agent transfer surface into a target tissue to thereby prevent or treat the disease. The target tissue may be intact tissue and the agent transfer surface may be configured to inhibit or prevent mechanical penetration of a surface of the target tissue when in contact with it during standard use of the device. The target tissue may be skin. The target tissue may be mucosal tissue and the agent transfer surface may be configured to inhibit or prevent mechanical penetration of an intact epithelial layer of the mucosal tissue during standard use of the device. The target tissue may also be ocular tissue and the agent transfer surface may be configured to inhibit or prevent mechanical penetration of an intact conjunctiva or corneal epithelial layer (or both) during standard use of the device.

The device may be a device according to any embodiment of the present invention.

The device may comprise a plurality of nanoscale channels extending partially or wholly through the agent carrier to the agent transfer surface enabling retention of the agent and/or transportation of the agent to the tissue. At least 80%, at least 90%, at least 95% or all of the plurality of nanoscale channels may have a maximum width (e.g. diameter) of at or below: 500 nm, 400 nm, 300 nm, 200 nm, 100 nm, 55 nm, 25 nm or 10 nm. The porosity of the plurality of nanoscale channels may account for up to 60%, 70%, 80% or 85% of the total agent carrier volume. The plurality of nanoscale channels may range in maximum width (e.g. diameter) from between 1 nm and 55 nm, from between 1 nm and 50 nm, from between 1 nm and 45 nm, from between 1 nm and 40 nm, from between 1 nm and 35 nm, from between 5 nm and 55 nm, from between 5 nm and 50 nm, from between 5 nm and 45 nm, from between 5 nm and 40 nm, from between 5 nm and 35 nm, from between 10 nm and 55 nm, from between 10 nm and 50 nm, from between 10 nm and 45 nm, from between 10 nm and 40 nm, and from between 10 nm and 35 nm. At a given cross section of the agent carrier, the plurality of nanoscale channels may be provided in an amount of at least: 5000, 6000, 7000, 8000, 9000, 10000, 11000, 12000, 13000, 14000, 15000, 20000, 25000, 50000, 75000 or 100000 channels/cm² of the agent carrier body,

The subject referred to in the above embodiments may be suffering from a disease, may be exhibiting one or more symptoms of a disease, or may be capable of contracting a disease.

The methods referred to in the above embodiments may comprise delivering the agent into or through any one or more of: epithelium, sub-epithelium, mucosa, sub-mucosa, mucous membrane vasculature, nasal septum, cornea, corneal epithelium, Bowman's membrane, corneal stroma, corneal endothelium, conjunctiva, Tenon's fascia, episclera, sclera, choroid, choriocapillaris, Bruch's membrane, retinal pigment epithelium, neural retina, retinal blood vessels, internal limiting membrane, vitreous humour, skin epidermis, skin dermis, teeth and nails, a component of the gastro-intestinal system, a component of the genito-urinary, a component of the reproductive system (e.g. vagina, uterus), a component of the respiratory system, a component of the ocular system, a component of the auditory system, an eye, an ear, testes and a lip.

The methods referred to in the above embodiments may comprise delivering the agent into a target tissue that is one of: mammalian target tissue, human target tissue or cell cultures.

In another embodiment, the present invention provides use of an agent in the manufacture of a medicament for preventing or treating a disease in a subject, wherein the medicament is loaded in a device comprising:

a piezoelectric substrate;

an agent carrier comprising an agent transfer surface for delivery of an agent into a target tissue, wherein the agent carrier comprises or is acoustically couplable to a piezoelectric substrate;

an electrode electrically couplable to the piezoelectric substrate; and

a controller electrically couplable to the electrode and configured to apply an electrical signal to the electrode to propagate acoustic waves on and/or in the piezoelectric substrate which is capable of delivering the agent from the device into the tissue to thereby prevent or treat the disease.

The device may be a device according to any embodiment of the present invention.

The device may comprise a plurality of nanoscale channels extending partially or wholly through the agent carrier to the agent transfer surface enabling retention of the agent and/or transportation of the agent to the tissue. At least 80%, at least 90%, at least 95% or all of the plurality of nanoscale channels may have a maximum width (e.g. diameter) of at or below: 500 nm, 400 nm, 300 nm, 200 nm, 100 nm, 55 nm, 25 nm or 10 nm. The porosity of the plurality of nanoscale channels may account for up to 60%, 70%, 80% or 85% of the total agent carrier volume. The plurality of nanoscale channels may range in maximum width (e.g. diameter) from between 1 nm and 55 nm, from between 1 nm and 50 nm, from between 1 nm and 45 nm, from between 1 nm and 40 nm, from between 1 nm and 35 nm, from between 5 nm and 55 nm, from between 5 nm and 50 nm, from between 5 nm and 45 nm, from between 5 nm and 40 nm, from between 5 nm and 35 nm, from between 10 nm and 55 nm, from between 10 nm and 50 nm, from between 10 nm and 45 nm, from between 10 nm and 40 nm, and from between 10 nm and 35 nm. At a given cross section of the agent carrier, the plurality of nanoscale channels may be provided in an amount of at least: 5000, 6000, 7000, 8000, 9000, 10000, 11000, 12000, 13000, 14000, 15000, 20000, 25000, 50000, 75000 or 100000 channels/cm² of the agent carrier body,

In another embodiment, the present invention provides use of an agent in the manufacture of a medicament for preventing or treating a disease in a subject, wherein the medicament is prepared for use in a device comprising:

a piezoelectric substrate;

an agent carrier comprising an agent transfer surface for delivery of an agent into a target tissue, wherein the agent carrier comprises or is acoustically couplable to a piezoelectric substrate;

an electrode electrically couplable to the piezoelectric substrate; and

a controller electrically couplable to the electrode and configured to apply an electrical signal to the electrode to propagate an acoustic wave on and/or in the piezoelectric substrate which is capable of delivering the agent from the device into the tissue to thereby prevent or treat the disease.

The device may be a device according to any embodiment of the present invention.

The device may comprise a plurality of nanoscale channels extending partially or wholly through the agent carrier to the agent transfer surface enabling retention of the agent and/or transportation of the agent to the tissue. At least 80%, at least 90%, at least 95% or all of the plurality of nanoscale channels may have a maximum width (e.g. diameter) of at or below: 500 nm, 400 nm, 300 nm, 200 nm, 100 nm, 55 nm, 25 nm or 10 nm. The porosity of the plurality of nanoscale channels may account for up to 60%, 70%, 80% or 85% of the total agent carrier volume. The plurality of nanoscale channels may range in maximum width (e.g. diameter) from between 1 nm and 55 nm, from between 1 nm and 50 nm, from between 1 nm and 45 nm, from between 1 nm and 40 nm, from between 1 nm and 35 nm, from between 5 nm and 55 nm, from between 5 nm and 50 nm, from between 5 nm and 45 nm, from between 5 nm and 40 nm, from between 5 nm and 35 nm, from between 10 nm and 55 nm, from between 10 nm and 50 nm, from between 10 nm and 45 nm, from between 10 nm and 40 nm, and from between 10 nm and 35 nm. At a given cross section of the agent carrier, the plurality of nanoscale channels may be provided in an amount of at least: 5000, 6000, 7000, 8000, 9000, 10000, 11000, 12000, 13000, 14000, 15000, 20000, 25000, 50000, 75000 or 100000 channels/cm² of the agent carrier body,

The medicament may be capable of inducing a mucosal immune response and/or a systemic immune response when administered through the device.

The agent of any of the above embodiments may comprise a solid or a combination of a solid and a liquid. The agent may comprise a solid comprising powder, granules, or a combination thereof. The agent may be lyophilised. The agent may comprise a therapeutic agent, a prophylactic agent, a diagnostic agent, a cosmetic agent, or any combination thereof. The agent may be selected from the group consisting of: a protein, a peptide, a polypeptide, an antibody (including monoclonal and polyclonal antibodies), an immunogenic agent, a vaccine, a biomimetic, a biosimilar, a biomaterial, a macromolecule, a small molecule, a sugar, a nucleic acid, a drug, a nanoparticle, and any combination thereof.

The agent of any of the above embodiments may be delivered to a target depth within the tissue and/or at a specific rate of delivery using the controller of the device to regulate the duration, frequency and/or amplitude of the acoustic waves propagated on and/or in the piezoelectric substrate of the device. The displacement amplitude at the tissue contact surface area of the agent carrier may be in a range of 100 to 1375 nm. The target tissue depth may be in a range of 10 μm to 5 mm.

Delivery of the agent into the target tissue according to any of the above embodiments may induce a mucosal immune response and/or a systemic immune response.

According to another embodiment of the present invention, there is provided a device, comprising:

a piezoelectric substrate;

a source of an agent on or acoustically couplable to the piezoelectric substrate; and

an electrode electrically couplable to the piezoelectric substrate;

a controller electrically couplable to the electrode and configured to apply an electrical signal to the electrode to propagate an acoustic wave on and/or in the piezoelectric substrate that is sufficient to deliver the agent from the source to under a surface of an area of tissue.

The device may be a device according to any embodiment of the present invention.

The device may comprise a plurality of nanoscale channels extending partially or wholly through the agent carrier to the agent transfer surface enabling retention of the agent and/or transportation of the agent to the tissue. At least 80%, at least 90%, at least 95% or all of the plurality of nanoscale channels may have a maximum width (e.g. diameter) of at or below: 500 nm, 400 nm, 300 nm, 200 nm, 100 nm, 55 nm, 25 nm or 10 nm. The porosity of the plurality of nanoscale channels may account for up to 60%, 70%, 80% or 85% of the total agent carrier volume. The plurality of nanoscale channels may range in maximum width (e.g. diameter) from between 1 nm and 55 nm, from between 1 nm and 50 nm, from between 1 nm and 45 nm, from between 1 nm and 40 nm, from between 1 nm and 35 nm, from between 5 nm and 55 nm, from between 5 nm and 50 nm, from between 5 nm and 45 nm, from between 5 nm and 40 nm, from between 5 nm and 35 nm, from between 10 nm and 55 nm, from between 10 nm and 50 nm, from between 10 nm and 45 nm, from between 10 nm and 40 nm, and from between 10 nm and 35 nm. At a given cross section of the agent carrier, the plurality of nanoscale channels may be provided in an amount of at least: 5000, 6000, 7000, 8000, 9000, 10000, 11000, 12000, 13000, 14000, 15000, 20000, 25000, 50000, 75000 or 100000 channels/cm² of the agent carrier body,

The piezoelectric substrate may comprise a single crystal piezoelectric material, a thin-film piezoelectric material, or any combination thereof.

The source may comprise the piezoelectric substrate, a channel, a reservoir, a body, or any combination thereof.

The agent may comprise a liquid, a solid, a powder, or a combination thereof.

The agent may comprise a therapeutic agent, a prophylactic agent, a diagnostic agent, a cosmetic agent, or any combination thereof.

The electrode may comprise an interdigital transducer, a plate electrode, an electrode layer, or any combination thereof.

The acoustic wave may comprise a surface acoustic wave, a lamb wave, or a combination thereof.

The controller may be further configured to generate an ultrasonic wave to frequency modulate the surface acoustic wave, the lamb wave, or any combination thereof.

The area of tissue may comprise epithelial tissue, sub-epithelial tissue, or any combination thereof.

Another embodiment of the present invention provides a method, comprising non-invasively delivering an agent to a controllable depth under a surface of an area of tissue using the device described above.

The method may comprise a prophylactic method, a therapeutic method, a diagnostic method, a cosmetic method, or any combination thereof.

A further embodiment of the present invention provides a method, comprising conferring one or both of mucosal or systemic immunity by delivering an agent to epithelial or sub-epithelial tissue using the device described above.

Without limitation, it will be recognised that the present invention relates at least in part to the following listed exemplary embodiments:

Embodiment 1. A device, comprising:

an agent carrier comprising an agent transfer surface for non-invasive delivery of an agent into a tissue, and a plurality of nanoscale channels extending partially or wholly through the agent carrier to the agent transfer surface enabling retention of the agent and/or transportation of the agent to the tissue, wherein the agent carrier comprises or is acoustically couplable to a piezoelectric substrate;

an electrode electrically couplable to the piezoelectric substrate; and

a controller electrically couplable to the electrode and configured to apply an electrical signal to the electrode to propagate an acoustic wave on and/or in the piezoelectric substrate which is capable of delivering the agent from the agent carrier into the tissue.

Embodiment 2. The device of embodiment 1, wherein at least: 50%, 60%, 70%, 80%, 90%, 95%; or all of the plurality of nanoscale channels, have a maximum width exceeding the maximum width of the agent by no more than: 1.2 fold (1.2×), 1.5-fold (×1.5), two-fold (×2), three-fold (×3), four-fold (×4), five-fold (×5), ten-fold (×10), twenty-fold (×20), thirty-fold (×30), forty-fold (×40), or fifty-fold (×50).

Embodiment 3. The device of embodiment 1 or embodiment 2, wherein at least: 50%, 60%, 70%, 80%, 90%, 95%; or all of the plurality of nanoscale channels, have a maximum width exceeding the maximum width of the agent by no more than: 1%, 2%, 3%, 4%, 5%, 10%, or 20%.

Embodiment 4. The device of any one of embodiments 1 to 3, wherein:

the plurality of nanoscale channels terminate as pores at the agent transfer surface; and the pores have a maximum width exceeding the maximum width of the agent by no more than: 1.2 fold (1.2×), 1.5-fold (×1.5), two-fold (×2), three-fold (×3), four-fold (×4), five-fold (×5), ten-fold (×10), twenty-fold (×20), thirty-fold (×30), forty-fold (×40), or fifty-fold (×50).

Embodiment 5. The device of any one of embodiments 1 to 4, wherein:

the plurality of nanoscale channels terminate as pores at the agent transfer surface; and

the pores have a maximum width exceeding the maximum width of the agent by no more than: 1%, 2%, 3%, 4%, 5%, 10%, or 20%.

Embodiment 6. The device of any one of embodiments 1 to 5, wherein at least: 50%, 60%, 70%, 80%, 90%, 95%; or all of the plurality of nanoscale channels, have a maximum width of below: 65 nm, 55 nm, 50 nm, 24 nm or 10 nm; or a maximum width of between 160 nm and 999 nm, 160 nm and 300 nm, 160 nm and 450 nm, 160 nm and 600 nm, 160 nm and 750 nm, 160 nm and 900 nm, or 160 nm and 999 nm.

Embodiment 7. The device of any one of embodiments 1 to 6, wherein the plurality of nanoscale channels range in maximum width (e.g. diameter) from between 1 nm and 55 nm, from between 1 nm and 50 nm, from between 1 nm and 45 nm, from between 1 nm and 40 nm, from between 1 nm and 35 nm, from between 5 nm and 55 nm, from between 5 nm and 50 nm, from between 5 nm and 45 nm, from between 5 nm and 40 nm, from between 5 nm and 35 nm, from between 10 nm and 55 nm, from between 10 nm and 50 nm, from between 10 nm and 45 nm, from between 10 nm and 40 nm, and from between 10 nm and 35 nm.

Embodiment 8. The device of any one of embodiments 1 to 7, wherein the porosity of the plurality of nanoscale channels accounts for up to 60%, 70%, 80% or 85% of the total agent carrier volume.

Embodiment 9. The device of any one of embodiments 1 to 8, wherein:

the plurality of nanoscale channels extend from the interior of the agent carrier body and terminate as pores at the agent transfer surface.

Embodiment 10. The device of any one of embodiments 1 to 9, wherein at least 80%, at least 90%, at least 95% or all of the pores have a maximum width below: 65 nm, 55 nm, 50 nm, 24 nm or 10 nm.

Embodiment 11. The device of any one of claims 1 to 10, wherein the plurality of nanoscale channels are provided in an amount of at least: 5000, 6000, 7000, 8000, 9000, 10000, 11000, 12000, 13000, 14000, 15000, 20000, 25000, 50000, 75000 or 100000 channels/cm² of the agent carrier body.

Embodiment 12. The device of any one of embodiments 1 to 11, wherein the plurality of nanoscale channels is fabricated using any one or more of: silicon, porated silicon, germanium, graphene, synthetic polymer or a combination thereof.

Embodiment 13. The device of any one of embodiments 1 to 11, wherein the plurality of nanoscale channels is fabricated using three-dimensional (3D) printing of a material selected from the group consisting of: polymeric material, metallic material, ceramic material, and any combination thereof.

Embodiment 14. The device of any one of embodiments 1 to 13, further comprising an acoustic generator capable of generating a secondary acoustic excitation frequency capable of modulating a primary acoustic excitation frequency generated by the piezoelectric substrate, wherein the secondary acoustic excitation frequency is less than or equal to the primary acoustic excitation frequency.

Embodiment 15. The device of any one of embodiments 1 to 14, wherein the device:

does not comprise an electrode for contacting the tissue surface, and/or

is not configured to utilise repulsive electromotive force to transport a charged agent into and/or through the tissue in contact with the agent transfer surface.

Embodiment 16. The device of any one of embodiments 1 to 15, wherein:

the agent carrier comprises the piezoelectric substrate,

the piezoelectric substrate comprises the agent transfer surface, and

the agent is present on the agent transfer surface.

Embodiment 17. The device of embodiment 16, wherein the agent is functionalised and/or lyophilised on the agent transfer surface.

Embodiment 18. The device of any one of embodiments 1 to 17, wherein the device is non-invasive, and the agent transfer surface does not comprise microneedles.

Embodiment 19. A method for delivering an agent to an internal layer within a target tissue, the method comprising:

contacting the target tissue with the agent transfer surface of the device of any one of embodiments 1 to 18, and

applying an electrical signal to the electrode of the device to propagate acoustic waves on and/or in the piezoelectric substrate of the device, and thereby deliver the agent from the agent transfer surface to the internal layer of the target tissue.

Embodiment 20. The method of embodiment 19, wherein the method comprises delivering the agent into or through any one or more of: epithelium, sub-epithelium, mucosa, sub-mucosa, mucous membrane vasculature, nasal septum, cornea, corneal epithelium, Bowman's membrane, corneal stroma, corneal endothelium, conjunctiva, Tenon's fascia, episclera, sclera, choroid, choriocapillaris, Bruch's membrane, retinal pigment epithelium, neural retina, retinal blood vessels, internal limiting membrane, vitreous humour, a component of the gastro-intestinal system, a component of the genito-urinary, a component of the reproductive system (e.g. vagina, uterus, testes), a component of the respiratory system, a component of the ocular system, a component of the auditory system, an eye, an ear, and a lip.

Embodiment 21. The method of embodiment 19 or embodiment 20, wherein:

the target tissue is intact tissue, and

the agent transfer surface is configured to inhibit or prevent mechanical penetration of a surface of the target tissue and to prevent piercing or destruction of the tissue by ultrasonic waves emanating from the device, when in contact with the tissue during standard use of the device.

Embodiment 22. The method of any one of embodiments 19 to 21, wherein the target tissue is mucosal tissue, or the eye.

Embodiment 23. The method of embodiment 22, wherein the mucosal tissue is intact, the agent transfer surface does not penetrate an intact epithelial layer of the mucosal tissue during standard use of the device, and wherein delivery of a therapeutically effective amount of the agent into the mucosal tissue induces an immune response in the subject.

Embodiment 24. The method of embodiment 23, wherein the immune response is at least a mucosal immune response.

Embodiment 25. The method of embodiment 24, wherein the mucosal immune response is induced by controlling the amount of agent delivered into an epithelial layer of the mucosal tissue, or into the epithelial and sub-epithelial layers of a mucous membrane.

Embodiment 26. The method of embodiment 23, wherein the immune response is a systemic immune response.

Embodiment 27. The method of embodiment 26, wherein delivery of the agent to induce a systemic immune response is by controlling the amount of agent delivered into and through the epithelial and sub-epithelial tissue.

Embodiment 28. The method of embodiment 22, wherein the target tissue is the eye, and the method comprises contacting the agent transfer surface with corneal epithelium and delivering a target amount of the agent into the cornea of the eye.

Embodiment 29. The method of embodiment 28, wherein:

the agent is delivered for the treatment of myopia or keratoconus,

the agent is a therapeutically effective amount of any one or more of riboflavin-5-phosphate sodium salt, glutaraldehyde, grape seed extract, and/or genipin, and

the method further comprises exposing the cornea to ultraviolet light following delivery of the therapeutic amount of the agent to the cornea for a time period sufficient to induce collagen crosslinking in the cornea.

Embodiment 30. The method of embodiment 29, further comprising repeating the delivery of the therapeutically effective amount and the exposure to ultraviolet light within 1, 2, 3, 4, 5, 6, 7, 14, 21, 28, 42 or 60 days.

Embodiment 31. The method of embodiment 28, wherein:

the agent comprises a therapeutic amount of the agent for treating a condition or disease upon delivery to the posterior segment of the eye,

and the therapeutically effective amount of the agent
is delivered through the corneal epithelium, Bowman's membrane, Corneal stroma, Descemet's membrane and Corneal endothelium, into aqueous humor,
circulates within the aqueous humor through the pupil and around the lens into the posterior chamber,
contacts one or more of: vitreous humor, ciliary body blood vessels, uveal blood vessels in the pars plana, and
is distributed via the choroidal vasculature to the posterior segment of the eye.

Embodiment 32. The method of embodiment 22, wherein:

the agent comprises a therapeutic amount of the agent for treating a condition or disease upon delivery to the posterior segment of the eye,

and the therapeutically effective amount of the agent
is delivered through the conjunctiva overlying the sclera, and the sclera,
enters the uveal tract of the eye,
is distributed via the choroidal vasculature to the choroid and retina in the posterior segment of the eye.

Embodiment 33. The method of embodiment 31 or embodiment 32, wherein the therapeutically effective amount of the agent comprises anti-Vascular Endothelial Growth Factor (anti-VEGF) agents, nucleic acids, and/or an anti-inflammatory drug, and is delivered for the treatment of Age Related Macular Degeneration, Diabetic Eye Disease, or Posterior Choroiditis.

Embodiment 34. The method of any one of embodiments 19 to 33, wherein propagating the acoustic wave comprises generating ultrasonic power in the range 0.05 to 5.25 Wcm⁻², or 0.05 to 0.7 Wcm⁻², for the delivery of the agents into the target tissue.

Embodiment 35. The method of any one of embodiments 19 to 34, comprising generating a primary acoustic excitation frequency on and/or in the piezoelectric substrate of less than 1 mHz, between 35 kHz and 50 kHZ, 35 kHz and 55 kHZ, or above 1 mHz.

Embodiment 36. The method of embodiment 35, further comprising generating one or more secondary acoustic excitation frequencies on and/or in the piezoelectric substrate to thereby modulate the primary acoustic excitation on and/or in the piezoelectric substrate.

Embodiment 37. The method of embodiment 36, wherein the secondary acoustic excitation frequency is less than or equal to the primary acoustic excitation frequency.

Embodiment 38. The device of any one of embodiments 1 to 18, wherein the delivering comprises transportation of the agent through the nanoscale channels by the acoustic waves to the agent transfer surface.

Embodiment 39. The device of any one of embodiments 1 to 18 or 38, wherein the delivering comprises continuous operation of the device over a time period of more than: one minute, two minutes, three minutes, four minutes, 5 minutes or 10 minutes.

Embodiment 40. The method of any one of embodiments 19 to 37, wherein the delivering comprises transportation of the agent through the nanoscale channels by the acoustic waves to the agent transfer surface.

Embodiment 41. The method of any one of embodiments to 19 to 37 or 40, wherein the delivering comprises continuous operation of the device over a time period of more than: one minute, two minutes, three minutes, four minutes, 5 minutes or 10 minutes.

Embodiment 42. The device of any one of embodiments 1 to 18, 38 or 39, wherein the device comprises an internal reservoir in fluid communication with the nanoscale channels and comprising some or all of the agent.

Embodiment 43. The method of any one of embodiments 19 to 37, 40 or 41, wherein the device comprises an internal reservoir in fluid communication with the nanoscale channels and comprising some or all of the agent.

### BRIEF DESCRIPTION OF DRAWINGS

Embodiments of the invention will now be described by way of example only with reference to the accompanying drawings, in which:

FIG. 1A shows a schematic cross-sectional block diagram of an applicator device according to one embodiment, that being applied to a tissue surface and provides an illustration of the overall components of one exemplary applicator device.

FIG. 1B shows a more detailed cross-sectional view of the agent carrier body of the embodiment shown in FIG. 1A.

FIG. 1C shows a similar agent carrier body to that of FIG. 1B that includes an ultrasonic transducer.

FIG. 2 provides a cross sectional block diagram of an embodiment of a handle assembly of the applicator device and its basic component parts.

FIG. 3 is a cross sectional view through an agent carrier that takes the form of a single use applicator tip.

FIGS. 4k, 4B, and 4C provide illustrations of various embodiments of a single layer agent carrier body with different nanoscale channel, and or reservoir arrangements.

FIG. 4D provides an illustration of an embodiment of a first surface and a tissue contact surface of a single layer agent carrier body.

FIG. 5 is a schematic diagram of a device according to one embodiment where an acoustic wave is applied to a solid having nanoscale fabricated features on it that contains the agent for drug delivery according to the present invention;

FIG. 6 is a schematic diagram of a device according to one embodiment where an acoustic wave is applied to a fluid couplant in contact with a solid having nanoscale fabricated features on it that contains the agent for drug delivery according to the present invention;

FIG. 7 is a schematic diagram of a device according to one embodiment where an acoustic wave is applied to a solid having nanoscale fabricated features on it that contains the agent for drug delivery according to the present invention.

FIG. 8 is a photograph of a series of silicon wafers each treated with different critical fabrication parameters.

FIG. 9 shows cross-section SEM images of the pSi tips prepared by applying an electrical current density to a silicon wafer of (a) 83 mA·cm⁻² for 150 min and (b) 50 mA·cm⁻² for 240 min, respectively. Insets: top-view SEM images of the pSi tips showing the opening of the nanopores after sacrificial layer dissolution (scale bar 50 nm).

FIG. 10 shows histograms of the pore size distribution for the pSi tips etched using a current density of (a) 83 mA·cm⁻² for 150 min, producing an average pore size of 21.9 nm (σ=12.7 nm), and (b) 50 mA·cm⁻² for 240 min with an average pore size of 16.2 nm (σ=8.3 nm), respectively.

FIG. 11 provides photographs of sessile water drops on (a) as prepared and (b) thermally oxidized pSi tips loaded with 1 μL of water. Surfaces (c) and (d) were treated using the two-step oxidation approach (i.e., thermal and ozone oxidation). Surface (c) was loaded with 7 μL of PBS solution, whereas surface (d) was loaded using 7 μL PBS solution containing ethanol (2.5%, v/v) showing a homogeneous diffusion of the solution into the porous scaffold.

FIG. 12 shows bright field, Cy5 fluorescence and merged CLSFM images of pSi membranes. (a) Control membrane tested with buffer, and (b) and (c) pSi membranes tested with fluorescently tagged Ab. Red arrows indicate the top surface of each membrane.

FIG. 13 shows CSLFM images of the (a) top and (b) open-ended bottom membrane surface detached from the crystalline silicon substrate. The pSi membrane was tested with a buffer solution containing the fluorescently tagged Ab.

FIG. 14 (a) Photographs of pSi tips and (b) experimental setup for ultrasound mediated delivery experiments.

FIG. 15 provides photographs of agarose hydrogel cubes tested with pSi tip loaded with FITC (0.1 mM) and applied for 30 s with (‘On’) and without (‘Off’) ultrasound: (a) and (c) top-views photographs of the agarose cubes under ambient and UV light, respectively; (b) cross-section photograph showing the diffusion of FITC into the medium and; (d) transverse intensity profile (in pixel counts) of agarose samples tested with and without ultrasound.

FIG. 16 (a) Top and (b) cross-section photographs of agarose hydrogel cubes tested with the pSi tip loaded with FITC-tagged Ab (1.3 μM) with (‘On’) and without (‘Off’) ultrasound application.

FIG. 17 (a) Photographs of the cadaver eye in the embedding medium matrix (top) and cryosection sample of the eye tissue prior to imaging analysis (bottom). Red rectangle encloses the ROI where Ab-loaded pSi tip was applied with ultrasound assistance; (b) fluorescence image showing the Cy5 emission of the ultrasound delivered Ab into the scleral tissue of the eye. Red arrows point out the length of the RIO tested with the pSi tip.

FIG. 18 (a) Photographs of the cadaver eye in the embedding medium matrix (top) cryosection sample of the eye tissue prior to imaging analysis (bottom). Red rectangle encloses the ROI where Ab-loaded pSi tip was applied without ultrasound assistance; (b) fluorescence image showing negligible Cy5 emission in the scleral tissue of the eye. Red arrows point out the length of the ROI tested with the pSi tip.

FIG. 19 shows an expected result where all Ga-68 is bound to Avastin with no evidence of antibody aggregation. A Non-labelled Avastin HPLC trace. B Ga-68 labelled Avastin trace showing a single radioactive peak with an identical elution profile to cold Avastin (retention time: 7.34 min). Size exclusion column with a mobile phase of 0.15 M phosphate buffer (pH 7) at 0.35 mLmin⁻¹.

FIG. 20 shows an example layout of data in accordance with (prophetic) embodiments of the invention.

FIG. 21 shows an example layout of data for activity at application site in accordance with (prophetic) embodiments of the invention.

FIG. 22 shows an example of an expected result of positive anti-human antibody fluorescent signal present within the deeper eye tissues post ultrasonic delivery of Avastin.

FIG. 23 shows (i) a pore histogram chart of the morphological features observed in (ii) the top-view SEM image of the porous surface of an exemplary device of the present invention.

FIG. 24 relative reflectivity spectrum for a pSi surface (a) in air and (b) with its pores filled with ethanol (EtOH). (c) FFT peak (amplitude) and EOT values of the pSi surface before and after filling the pores. pSi surface was anodised using a current density of 50 mA·cm⁻² for 120 s.

FIG. 25 shows top and cross-sectional SEM images and pore size histograms of pSi surfaces prepared by applying an electrical current density to a silicon wafer of (a-c) 57 mA·cm² for 180 min, (d-f) 64 mA·cm² for 180 min, (g-i) 79 mA·cm² for 120 min, and (j-l) 100 mA·cm⁻² for 120 min, respectively.

FIG. 26 shows calibration curves for (a) the etching rate (Vₑ) and (b) porosity based on the calculated physical properties of the surfaces listed in Table 6. Experimental data were fitted using linear regression (dashed line).

FIG. 27 shows structural stress caused by the porous skeleton volume expansion during the annealing treatment (400° C., 1 h in air) for (a) Surface 1, (b) Surface 2, and (c) Surface 3 (surfaces fabricated as listed in Table 5).

FIG. 28 shows pore availability for admission of Avastin. (i) Pore histogram chart of the morphological features observed in (ii) the top-view SEM image of the porous surface.

FIG. 29a-c shows data from driving one type of transducer in the device at 27 Vpp.

FIG. 30a-c shows data from driving one type of transducer in the device at 30 Vpp.

FIG. 31a-b shows the estimated irradiance of the tip in W/Cm².

FIG. 32 shows an image of multiple substrates in a wafer configuration before dicing.

FIG. 33 is a graph showing results of an absorbance analysis—Standard insulin induced proliferation in cells.

FIG. 34 is a histology image of a negative control confirming the low level autofluorescence emanating from erythrocytes in a section stained with DAPI alone.

FIG. 35 is a histology image of a test section presented with areas of yellow colour which was determined to provide confirmation of immunoreactivity towards human-Ig (and thus Avastin).

FIG. 36 (a-h) are histology images taken from Rabbit 1, right eye—displaying some staining within the conjunctiva.

FIG. 37 (a-j) are histology images taken from Rabbit 1, left eye.

FIG. 38 (a-l) are histology images taken from Rabbit 2, right eye.

FIG. 39 (a-m) are histology images taken from Rabbit 2, left eye.

FIG. 40 (a-r) are histology images taken from Rabbit 3, right eye.

FIG. 41 (a-h) are histology images taken from Rabbit 3, left eye.

FIG. 42 shows expected results of a prophetic Example of the present application. Coomassie Brilliant Blue staining of saline buffer samples separated by SDS PAGE. Saline buffer samples containing BSA as contained in the device tip operated with ultrasound for increasing durations. “C” Control represents a Saline buffer sample containing BSA contained in the device tip for 90 seconds without ultrasound. “N” is known protein standard. Tip outer diameter 9.55 mm. All tips contained 2 mg/mL BSA.

FIG. 43 shows expected results of a prophetic Example of the present application. Coomassie Brilliant Blue staining of saline buffer samples separated by SDS PAGE. Saline buffer samples containing BSA as contained in the device tip operated with ultrasound for increasing durations. “C” Control represents a Saline buffer sample containing BSA contained in the device tip for 90 seconds without ultrasound. “N” is known protein standard. Tip outer diameter 9.55 mm. All tips contained 4 mg/mL BSA.

FIG. 44 shows expected results of a prophetic Example of the present application. Coomassie Brilliant Blue staining of saline buffer samples separated by SDS PAGE. Saline buffer samples containing Avastin as contained in the device tips operated with ultrasound for increasing durations. “C” Control represents a Saline buffer sample containing Avastin contained in the device tip for 90 seconds without ultrasound. Tip outer diameter 9.55 mm. All tips contained 25 mg/mL Avastin.

FIG. 45 shows Ga-68 is bound to Avastin with no evidence of free Ga-68 and <5% antibody aggregation. A) Radiolabelled Avastin HPLC trace (retention time 9:04 min). B) UV absorption trace of Avastin (retention time 8:49 min). The small difference in the retention time is due to the placement of the detectors in series. C) RadioTLC trace showing a single peak at the origin due to radiolabelled Avastin. Free Ga-68 elutes with the solvent front.

HPLC utilised a size exclusion column with a mobile phase of 0.15 M phosphate buffer (pH 7) at 0.35 mLmin-1. RadioTLC utilised a mobile phase of 1 M sodium citrate (pH 5.5).

FIG. 46 shows regions of interest for quantitation of PET results. The MRI, CT and PET images were manually overlaid and ROIs incorporating the positive PET region for each eye (red and light green) and the whole eye (green and pink) were calculated and drawn. Above is a single representative coronal slice from rabbit 3 showing the ROIs.

FIG. 47 shows Avastin deposition is consistent during the 60 minutes of PET imaging. The mean of the PET signal in the total positive PET regions of interest for the left and right eyes from Rabbit 3 is plotted in 10 minute intervals over the course of the 60 minute imaging time. The blue circles indicate the mean activity from the left eye which was treated with 5 minutes of ultrasound and the black symbols indicate the mean activity from the right eye which was treated with the device for 5 minutes with no ultrasound applied. This is independent of any area measurement.

FIG. 48 shows Avastin deposition is increased following ultrasound and remains at the upper quadrant of the eye. The image shows fused CT, MRI and PET images from a coronal view from the top of the head downwards. The 4 slices from left to right start at the uppermost portion of the eyes and progress at 1 mm intervals to the central area of the eye. All rabbit treatments are as in Table 2 and show the data from the 50-60 minute time post application for each rabbit.

FIG. 49 shows ultrasound increases deposition of Avastin on the eye. A: The area of the Eye that showed PET signal >2 times background, B: The total activity of Avastin at each eye was quantitated from the mean Bq/ml signal times the area in the total ROI for each eye from all three rabbits. C: The total amount of Avastin (Bq/ml) remaining at the eye multiplied by the specific activity of the Ga68 labelled Avastin.

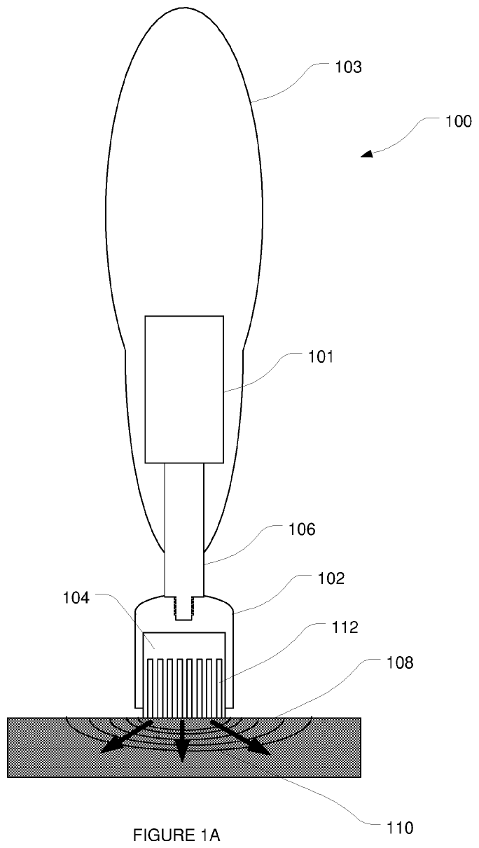

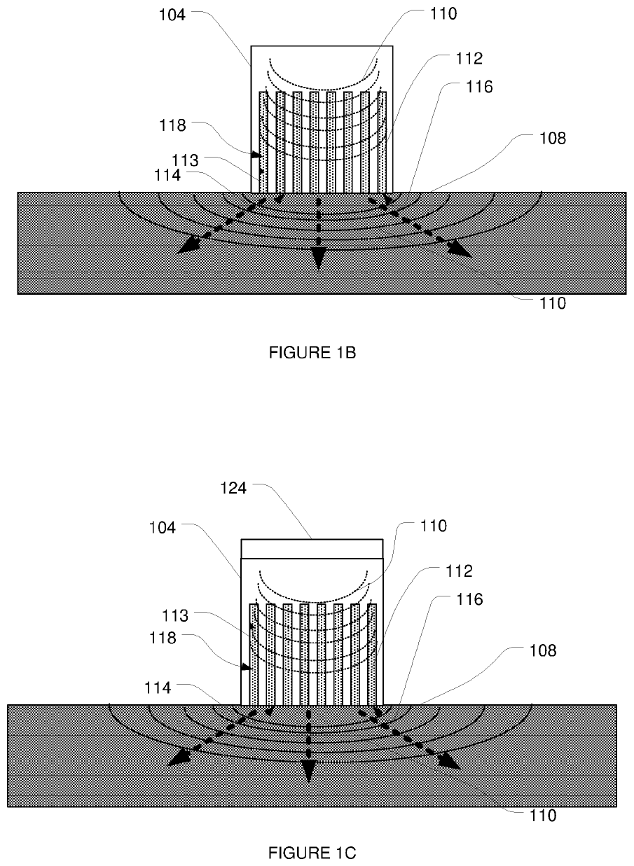

### DESCRIPTION OF EMBODIMENTS

The present invention provides devices and methods for the non-invasive delivery of an agent into tissue. The devices propagate acoustic waves from a piezoelectric transducer which is used as a transportation stimulus to deliver an agent into the tissue, where such acoustic waves do not cause piercing of the tissue or otherwise destroy any layer of the tissue. The device comprises a plurality of nanoscale channels, and an agent transfer surface of the device that does not mechanically penetrate, pierce or otherwise destroy any layer of tissue to which it is applied. Nor does the device acoustically pierce or destroy any part of the tissue. As used herein, the term “nanoscale” will be understood to mean less than 1000 nM and at or more than 1 nM such as, for example, between 1 nM and 2 nM, between 2 nM and 50 nM, between 50 nM and 100 nM, between 1 nM and 200 nM, between 1 nM and 300 nM, between 1 nM and 400 nM, between 1 nM and 500 nM, between 1 nM and 600 nM, between 1 nM and 700 nM, between 1 nM and 800 nM, between 1 nM and 900 nM, between 1 nM and 999 nM, between 100 nM and 999 nM, between 100 nM and 900 nM, between 200 nM and 999 nM, between 300 nM and 999 nM, between 400 nM and 999 nM, between 500 nM and 999 nM, between 600 nM and 999 nM, between 700 nM and 999 nM, between 800 nM and 999 nM, between 300 nM and 700 nM, between 400 nM and 800 nM, between 500 nM and 700 nM.

As used herein, the term “non-invasive” will be understood to mean that the device or method of delivering agents into tissue does not mechanically penetrate, pierce or destroy any part of the tissue, or acoustically pierce or destroy any part of the tissue.

Devices

Devices according to the present invention generally comprise an agent carrier which may comprise or be acoustically couplable to a piezoelectric substrate. The agent carrier also comprises an agent transfer surface for delivery of an agent into a tissue. The devices may also comprise an electrode electrically couplable to the piezoelectric substrate and a controller electrically couplable to the electrode. The controller may be configured to apply an electrical signal to the electrode to propagate an acoustic wave on and/or in the piezoelectric substrate which is capable of delivering the agent from the agent transfer surface into the tissue.

The acoustic wave may temporarily increase the permeability of a tissue in contact with the agent transfer surface of the device to thereby facilitate the entry of agent into the tissue. Without being limited by theory, the mechanisms for the entry of the agent into the tissue may include cavitation, fluidic jetting, physical vibration of cells making their surface membranes more permeable, and opening the inter-cellular spaces and cell to cell complexes whose adhesions hold adjacent cell walls together. The agent loaded in the device may be transported by, released by, and actively delivered into and/or through the tissue solely by virtue of the acoustic wave generated during operation of the device.

A device for drug delivery according to embodiments of the present invention may generally comprise an electroacoustic transducer on a piezoelectric substrate. The electroacoustic transducer may be controlled by a controller. A source of an agent may be fluidly couplable to, or physically contactable with, tissue 18, and acoustically couplable to the piezoelectric substrate 14. Further or alternatively, the source 16 of the agent may comprise the piezoelectric substrate 14 itself (i.e., the agent is disposed directly on the piezoelectric substrate 14). The source 16 may comprise a fluid comprising the agent, for example, a liquid containing the therapeutic agent.

The primary acoustic excitation frequency and power on and/or in the piezoelectric substrate of the device may depend on the piezoelectric transducer used, target depth of delivery and in some embodiments may exceed 1 mHz and/or 0.5 cm Wcm⁻². Supplementary, alternative or otherwise additional acoustic excitation frequencies of any wave type (including square, sine sawtooth) capable of modulating the primary acoustic excitation on and/or in the piezoelectric substrate may also be used and, for example may be less than (including in the range of 20 kHz-60 kHz) or equal to the primary acoustic excitation frequency.

Referring to FIG. 6, one embodiment of the device 10 may further comprise a fluid couplant 22 interposed between the fluid source 16 and the piezoelectric substrate 14.

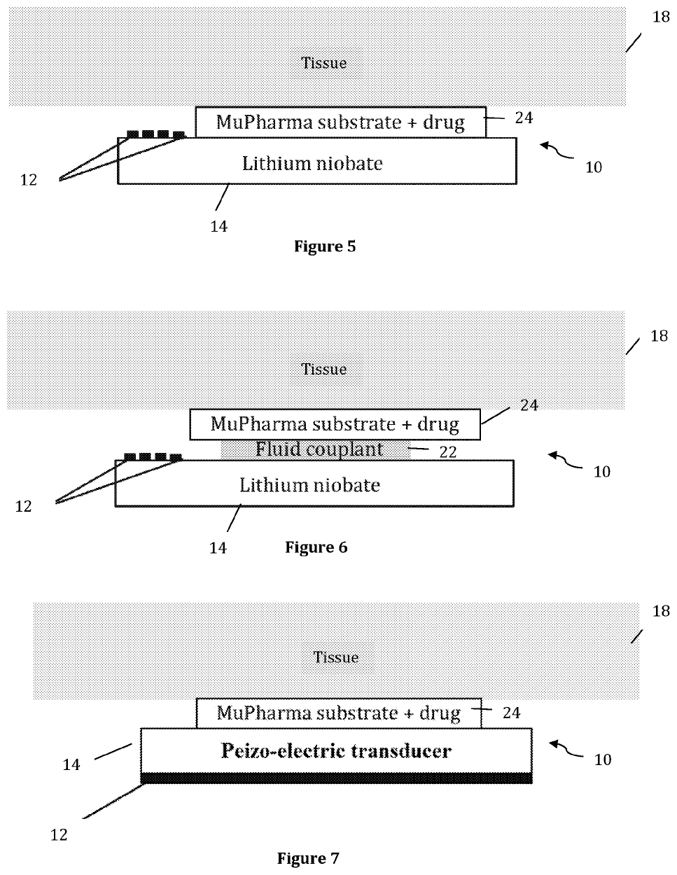

The electroacoustic transducer 12 may comprise interdigital transducers (IDTs), plate electrode, or an electrode layer. The piezoelectric substrate 14 may, for example, comprise a lithium niobate (LiNbO₃) substrate. The controller may, for example, be a programmable microcontroller. As illustrated in FIGS. 5 to 7, the agent 16 may be contained in a reservoir, such as a nanoscale fluidic reservoir or fluid nanoscale channels formed on an appropriate substrate (e.g. silicon, porous silicon, germanium, polymeric material). The nanoscale channels may extend from a reservoir of the agent partially or wholly through the device. Other alternative or equivalent materials, components and arrangements may also be used for the electroacoustic transducer 12, the piezoelectric substrate 14, the fluid source 16, and the controller.

The controller of the device 10 may be configured to apply signals (which may include RF signals) to the electroacoustic transducer 12 to controllably generate acoustic waves that fluidly couple with and drivingly transport, the agent to controllably deliver the agent to and into tissue 18. The controllable delivery of the agent across an epithelial membrane 18 may elicit a systemic immune response or a mucosal immune response (or both) in a subject. Preferably, at least a mucosal immune response is induced, and optionally a systemic immune response is also induced.

The acoustic waves generated by the device 10 may have a frequency corresponding to the resonant frequency of the piezoelectric substrate 14. The acoustic waves may comprise Rayleigh waves or bulk acoustic waves such as flexural, plate (e.g., Lamb) or thickness mode waves. The controller may be configured to control frequency or amplitude of the acoustic waves to control depth or rate of delivery of the therapeutic agent. The delivery depth may be in a range of 10 μm to 5 mm, for example, the depth of any of epithelial, dermal, intradermal, subdermal, mucosal epithelial, intramucosal, and submucosal tissue.

The device 10 in addition to generating a megahertz or higher range acoustic wave frequency may further comprise an acoustic frequency generator (not shown) to simultaneously, either continuously or intermittently, generate another acoustic frequency signal to modulate the megahertz or higher range acoustic wave frequency. The controller may further comprise a kilohertz range acoustic frequency generator. The kilohertz range acoustic frequency signal may have a frequency in a range of 1 Hz to 100 kHz. While it is not intended to be bound to any particular theory, it is believed that the modulation of the device megahertz or higher range frequency by a kilohertz range acoustic frequency signal may enhance, permit or otherwise facilitate the megahertz or higher range acoustic frequency signal mediated delivery of the agent to certain depths of tissue.

The epithelial membrane 18 may form part of a subject's mouth, rectum or other parts of the gastro-intestinal system, genito-urinary and reproductive system including the vagina and uterus, respiratory system, skin, conjunctiva, eye and ocular system and the ear and auditory system. The subject may be a human or an animal.

In some embodiments of the present invention, the agent carrier of the device may comprise a number or a network of nanoscale channels surrounded by rigid walls for retention and/or delivery of various agents. The agent carrier of the device may include more than: 10000, 20000, 30000, 40000, 50000, 750000, 100000, 200000, 300000, 400000, 500000, 600000, 700000, 800000, 900000; individual nanoscale channels. The nanoscale channels may be in fluid communication with a reservoir of the agent and extend partially or wholly through the device to its agent transfer surface. The nanoscale channels may extend from within the interior of the agent carrier to the agent transfer surface of the agent carrier.

At a given cross section of the agent carrier, the plurality of nanoscale channels may be provided in an amount of at least: 5000, 6000, 7000, 8000, 9000, 10000, 11000, 12000, 13000, 14000, 15000, 20000, 25000, 50000, 75000 or 100000 channels/cm² of the agent carrier body,

The nanoscale channels may range in maximum width (e.g. diameter) from between 1 nm to 999 nm, 2 nm to 999 nm, 5 nm to 999 nm, 10 nm to 999 nm, 50 nm to 999 nm, or 100 nm to 999 nm (e.g. 10 nm-800 nm, 10 nm-700 nm, 10 nm-600 nm, 10 nm-500 nm, 10 nm-400 nm, 10 nm-300 nm, 10 nm-200 nm, 10 nm-100 nm, 10 nm-75 nm, 10 nm-50 nm, 10 nm-25 nm; 50 nm-800 nm, 50 nm-700 nm, 50 nm-600 nm, 50 nm-500 nm, 50 nm-400 nm, 50 nm-300 nm, 50 nm-200 nm, 50 nm-100 nm, 50 nm-75 nm, 100 nm-900 nm, 100 nm-800 nm, 100 nm-700 nm, 100 nm-600 nm, 100 nm-500 nm, 100 nm-400 nm, 100 nm-300 nm, 100 nm-200 nm, 100 nm-150 nm, 200 nm-900 nm, 200 nm-800 nm, 200 nm-700 nm, 200 nm-600 nm, 200 nm-500 nm, 200 nm-400 nm, 200 nm-300 nm, 200 nm-250 nm, 300 nm-900 nm, 300 nm-800 nm, 300 nm-700 nm, 300 nm-600 nm, 300 nm-500 nm, 300 nm-400 nm, 300 nm-350 nm, 400 nm-900 nm, 400 nm-800 nm, 400 nm-700 nm, 400 nm-600 nm, 400 nm-500 nm, 500 nm-900 nm, 500 nm-800 nm, 500 nm-700 nm, 500 nm-600 nm), when measured transverse to the direction of delivery of the agent.

Additionally or alternatively, the nanoscale channels may have a length of between approximately 0.3 mm to 3 mm (e.g. 0.5 mm. 0.75 mm, 1 mm, 1.25 mm, 1.5 mm, 1.75 mm, 2 mm. 2.25 mm, 2.5 mm, 2.75 mm, between 0.5 mm and 3 mm, between 0.5 mm and 2.5 mm, between 0.5 mm and 2 mm, between 1 mm and 3 mm, or between 1.5 mm and 3 mm). Any suitable cross-sectional and/or longitudinal geometry can be employed (e.g. cylindrical, conical etc.).

The nanoscale channels may terminate as pores at the agent transfer surface. During use of the device the agent may travel through the nanoscale channels where it egresses through the pores of the agent transfer surface and into the tissue with which the agent transfer surface is in contact. A wide variety of shapes and sizes of pores may be utilised. The pores may, for example, be in the order of 1 nm to 999 nm in width, or 2 nm to 999 nm in width. The nanoscale channels may extend from the pores in the agent transfer surface at least partially or fully through the agent carrier body.

The agent transfer surface may include of more than: 10000, 20000, 30000, 40000, 50000, 750000, 100000, 200000, 300000, 400000, 500000, 600000, 700000, 800000, 900000; individual pores.

According to some embodiments of the present invention, the nanoscale channels and/or pores of the device can be fabricated to a width according to the size of the agent to be delivered, thereby improving and/or maximising the surface area (which can impart ultrasound to tissue) to volume (which can hold more agent) ratio of the device. This in turn may reduce the power (and in turn the heat and stress on the target tissue) needed to achieve the desired sonophoretic effect during agent delivery into tissue.

By way of non-limiting example, the maximum width of the nanoscale channels (e.g. 50%, 70%, 80%, 85%, 90%, 95% or all the nanoscale channels of the device) and/or the pores (e.g. 50%, 70%, 80%, 85%, 90%, 95% or all the pores of the device) may exceed the maximum width of the agent by no more than 1.2 fold (1.2×), no more than 1.5-fold (×1.5), no more than two-fold (×2), no more than three-fold (×3), no more than four-fold (×4), no more than five-fold (×5), no more than ten-fold (×10), no more than twenty-fold (×20), no more than thirty-fold (×30), no more than 40-fold (×40), no more than fifty-fold (×50), no more than 100-fold, no more than 500-fold, or no more than 1000-fold.

Additionally or alternatively, the maximum width of the nanoscale channels (e.g. 50%, 70%, 80%, 85%, 90%, 95% or all the nanoscale channels of the device) and/or pores (e.g. 50%, 70%, 80%, 85%, 90%, 95% or all the pores of the device) may exceed the maximum width of the agent by no more than 1%, no more than 2%, no more than 3%, no more than 4%, no more than 5%, no more than 10%, no more than 20%, no more than 30%, no more than 40%, or no more than 50%.

As referred to herein, the “maximum width” of a given nanoscale channel will be understood to be the distance between the two most spatially separated points within a horizontal plane oriented perpendicular to the central vertical axis of the channel. In the context of a nanoscale channel of non-uniform width, the spatially separated points of the horizontal plane are measured where the channel is widest.

As referred to herein, the “maximum width” of a given pore at the agent transfer surface of the device/agent carrier will be understood to be the distance between the two most spatially separated points on the external perimeter of the pore.

As referred to herein, the “maximum width” of a given agent will be understood to be the distance between the two most spatially separated points in the two- or three-dimensional structure of the agent.

In some embodiments, at least 26%, at least 30%, at least 40%, at least 52%, at least 60%, at least 70%, at least 80%, at least 90%, at least 95% or all of the plurality of nanoscale channels may have a maximum width (e.g. diameter) of below 66 nm, below 55 nm, below 50 nm, below 24 nm or below 10 nm.

Devices according to the present invention may comprise a unitary agent carrier. Alternatively, the agent carrier may be formed from a plurality of layers assembled together in a stacked fashion. The stack of layers may comprise an agent transfer surface layer, and at least one other layer. The agent transfer surface layer may have holes extending through it to define at least a portion of nanoscale channels in the body. In some embodiments a plurality of the layers has holes formed therein to enable agent to be transported from one layer to the next. Holes formed in one layer of the plurality of layers may be partially or completely aligned with holes in an adjacent layer so that a plurality of holes in a plurality of layers cooperate to form the nanoscale channels. In some embodiments the holes decrease in diameter and increase in number from the first layer to the tissue-containing layer. The nanoscale channels may have a varying cross-section along their length.

In some embodiments one or more reservoirs for storing the agent is partially or completely formed in the agent carrier of the device. The agent reservoir/s may comprise a void formed within the agent carrier body. Additionally or alternatively, the agent reservoir/s may be separate component/s in fluid communication with the agent carrier body. In some embodiments, the agent reservoir/s may be in fluid communication with one or more pores existing in the agent transfer surface. Additionally or alternatively, the reservoirs may be in fluid communication with one or more of the nanoscale channels. The nanoscale channels may extend partially or wholly through the device. The nanoscale channels and/or agent reservoir/s are generally defined by internal exposed surfaces within the agent carrier body. The internal exposed surfaces may be configured to possess predetermined hydrophilic, hydrophobic, and/or electro-conductive properties (e.g. chemical functionalisation of channel, reservoir and/or protrusion walls using, for example, materials such as polymers, natural polysaccharides, antibodies, peptides, tunable surfactants, alkyl chains and the like).

Any suitable material may be used to fabricate the nanoscale delivery devices of the present invention, non-limiting examples including silicon, porated silicon, germanium, graphene, polymeric material. In some embodiments three-dimensional (3D) printing may be used to produce the nanoscale delivery devices.

Methods for Agent Delivery

Embodiments of the present invention provide methods and related devices that are useful for ultrasound mediated targeted drug delivery. Advantageously, embodiments of the ultrasound devices of the invention may be used in methods of treatment of diseases or disorders, or used in methods of immunisation to elicit or stimulate immune responses.

Embodiments of the present invention involve subjecting an agent to an acoustic excitation to controllably deliver an agent to a preferred depth range in tissues. The agent can be a fluid or carried in a fluid medium, e.g. by being dissolved, suspended or dispersed in a fluid medium, such as water, oil, an emulsion, a gel or the like. The agent can also be in a solid form such as a powder. The agent can be housed within, and delivered from, a variety of materials.

In certain embodiments, the acoustic excitation may enhance penetration of the agent into the tissue by among other things, increasing the rate or depth (or both) of movement of an agent into tissue that would otherwise without the acoustic excitation, diffuse into tissue at a slower rate or to a lesser depth (or both). The acoustic excitation may alternatively permit or enable penetration of the agent into the tissue by among other things, enabling the movement of an agent into tissue that would otherwise, without the acoustic excitation, not be able to move into tissue at all or would diffuse in amounts less than that required to obtain the desired effect.

Embodiments of the present invention utilises among other things, drug-containing devices utilising acoustic wave devices comprising a piezoelectric material to produce a surface (SAW) and/or bulk (BAW) acoustic wave by utilising one or more acoustic frequencies, that are applied directly to target tissue for the purpose of delivering drugs primarily to specific groups of target cells located at specific depths in or near target tissue. The energy imparted to molecules or particles contained in such devices by acoustic waves alone or acoustic waves modulated by other frequencies facilitates their delivery to the target tissue cells that lie in, or immediately below, the epithelial surface.

Direct apposition of the drug containing surface of the device to mucosal tissues, serves to mechanically minimise contact with mucous and enzymes that are resident on the surface of such tissue and retard the inflow of mucous and enzymes from surrounding areas. This ensures that the dose is delivered accurately and minimises the problems associated with local mucous drug clearance and local enzymatic degradation. It therefore solves one or more of the problems encountered and associated with intranasal and pulmonary mucosal drug delivery by vapors and sprays.

The agent to be delivered can include one or more molecules or particles or one or more molecules and particles in any combination. To give but a few examples, the agent can include chemically synthesised substances, biologics like proteins, amino acids, peptides, polypeptides, vaccines, nucleic acids, monoclonal and polyclonal antibodies, as well as nanoparticles or molecular machines. In preferred embodiments the agent is a pharmaceutical or pharmaceutical composition. The pharmaceutical or one or more active pharmaceutical components of a pharmaceutical composition may be, without limit, any one of: a synthesised compound, a naturally occurring compound, or a biopharmaceutical. The purpose of the delivery of the pharmaceutical or pharmaceutical composition to the biological tissues can be for any desired clinical reason including: treating, curing or mitigating a disease, condition, or disorder; attenuating, ameliorating, or eliminating one or more symptoms of a particular disease, condition, or disorder; preventing or delaying the onset of one or more of a disease, condition, or disorder or a symptom thereof; diagnosing a disease, condition, or disorder, or any agent intended to affect the structure or any function of the body. In other embodiments the agent can be an agent used for cosmetic purposes such as for cleansing, beautifying, promoting attractiveness, or altering the appearance of the body. The agent could also be a marker agent used for creating human or machine perceptible makings, e.g. ink or other. Other types of agents may also be used.

As demonstrated in the Examples herein, the inventors have demonstrated delivery of the angiogenis inhibitor Avastin (bevacizumab) into target tissue using the device and methods of the present invention. Given the known difficulties of topical Avastin delivery and the successful outcomes demonstrated using the device and methods described herein, the skilled addressee will readily acknowledge that the delivery of other agents into various tissues using the device and methods is both achievable and predictable.

Additionally, the Examples herein demonstrate the delivery of Avastin (bevacizumab) into the conjunctiva, an ocular mucosal tissue. The skilled person will acknowledge that it is therefore both achievable and predictable that the device and methods described herein can be used to deliver agents into other ocular tissues and other mucosal tissues, such as, for example, the buccal mucosa.

The acoustic excitation is the driving force for moving the agent through and/or from the device, and may enhance or enable the penetration of the agent from the device into tissue.

In preferred embodiments, the tissue can be any human or animal biological tissue, including mucous membranes, skin, nails and teeth. Preferably, the tissue is oral mucosa or ocular tissue. In other embodiments, the tissue is any plant tissue.

The delivery depth of the agent into tissue may, for example, be in a range of 10 μm to 5 mm. Accordingly, the delivery depth of the agent into tissue may be in a range of 50 μm to 5 mm, 100 μm to 5 mm, 200 μm to 5 mm, 300 μm to 5 mm, 400 μm to 5 mm, 500 μm to 5 mm, 600 μm to 5 mm, 700 μm to 5 mm, 800 μm to 5 mm, 900 μm to 5 mm, 1 mm to 5 mm, 2 mm to 5 mm, 3 mm to 5 mm, 4 mm to 5 mm, 10 μm to 4 mm, 50 μm to 4 mm, 100 μm to 4 mm, 200 μm to 4 mm, 300 μm to 4 mm, 400 μm to 4 mm, 500 μm to 4 mm, 600 μm to 4 mm, 700 μm to 4 mm, 800 μm to 4 mm, 900 μm to 4 mm, 1 mm to 4 mm, 2 mm to 4 mm, 3 mm to 4 mm, 10 μm to 4 mm, 50 μm to 4 mm, 100 μm to 4 mm, 200 μm to 4 mm, 300 μm to 4 mm, 400 μm to 4 mm, 500 μm to 4 mm, 600 μm to 4 mm, 700 μm to 4 mm, 800 μm to 4 mm, 900 μm to 4 mm, 10 μm to 3 mm, 50 μm to 3 mm, 100 μm to 3 mm, 200 μm to 3 mm, 300 μm to 3 mm, 400 μm to 3 mm, 500 μm to 3 mm, 600 μm to 3 mm, 700 μm to 3 mm, 800 μm to 3 mm, 900 μm to 3 mm, 10 μm to 2 mm, 50 μm to 2 mm, 100 μm to 2 mm, 200 μm to 2 mm, 300 μm to 2 mm, 400 μm to 2 mm, 500 μm to 2 mm, 600 μm to 2 mm, 700 μm to 2 mm, 800 μm to 2 mm, 900 μm to 2 mm, 10 μm to 1 mm, 50 μm to 1 mm, 100 μm to 1 mm, 200 μm to 1 mm, 300 μm to 1 mm, 400 μm to 1 mm, 500 μm to 1 mm, 600 μm to 1 mm, 700 μm to 1 mm, 800 μm to 1 mm, or 900 μm to 1 mm.

The controlled delivery of the therapeutic agent across an epithelial membrane may elicit an immune response in a subject. The immune response induced in these aspects of the invention can be any one of a mucosal immune response, a systemic immune response, or both.

The acoustic excitation may comprise surface acoustic waves, bulk acoustic waves (e.g., flexural, plate (e.g., Lamb), or thickness mode waves), or combinations thereof.

To control depth or rate of delivery of the agent, the device may further comprise controlling operating parameters including (but not limited to) any one or more of the following:
application pressure;
acoustic frequency;
acoustic power level;
acoustic waveform;
acoustic application duration;
acoustic application duty cycle;
acoustic direction;
the material that houses the drug; and/or
the characteristics and ultrastructure of the agent transfer surface of the material

In some embodiments, the operational parameters are selected to deliver a chosen amount of agent to a selected depth within tissue. The person skilled in the art will appreciate that the optimal operational parameters needed to achieve a desired effect or response by application of agent to specific types of tissue can be determined by any combination of laboratory testing, other non-clinical means and by clinical investigations in animal models and human subjects.

Another way to control the depth or rate of delivery in the case of bulk transduction of the agent is for the device to include using a stack of one or more of each type of acoustic wave generating devices which serves to increase vibration amplitude and thus energy and power.

The methods of the present invention may involve delivering the agent to or beyond any one or more of the following tissues or tissue layers:

Mucous Membrane:
Epithelium
Sub-epithelium (lamina propria)

Mucosa:
Sub-mucosa
Mucous membrane vasculature

Cornea:
Corneal epithelium
Bowman's membrane
Corneal stroma
Descemet's membrane
Corneal Endothelium

Conjunctiva

Tenon's Fascia

Episclera

Sclera

Choroid

Choriocapillaris

Bruch's membrane

Retinal Pigment Epithelium

Neural retina

Retinal blood vessels

Internal Limiting Membrane

Vitreous humour

Aqueous humour

Skin
Epidermis
Dermis
Blood vessels

Teeth; and/or

Nails.

The target delivery site of a tissue utilised in the methods of the present invention may be defined as either being a particular layer or layers of a tissue, or alternatively be defined as a depth range. For example, the delivery of the agent may be defined in terms of being delivered to the Bowman's membrane of the cornea (i.e. a layer) or may be defined in terms of being delivered to a depth of approximately 5 to 150 (i.e. a depth range). The skilled person would be aware of what depth any given target layer is in any given tissue.

Delivery of Agents to a Mucosal Surface for Inducing an Immune Response

The immune response induced in these aspects of the invention can be a mucosal immune response, a systemic immune response, or both. Preferably, at least a mucosal immune response is induced, and optionally a systemic immune response is also induced. It is considered that by selectively configuring the operational parameters of the agent applicator presently described, the amount of agent delivered to a selected depth or one or more layers of a tissue may be controlled.

For example, in some embodiments of the invention, there is provided delivery of the agent to induce at least a mucosal immune response by controlling the delivery of the agent such that the majority of the agent is delivered into the epithelial and sub-epithelial layer of the mucous membrane. Accordingly, in some embodiments of the invention, delivery of the agent induces at least a mucosal immune response. The agent may be applied using the operational parameters described herein, and preferably a sufficient dose of agent remains resident in the mucous membrane, at least temporarily, in order to induce an immune response in the mucous membrane. More specifically, a sufficient dose of agent remains resident at least temporarily in one or more of the epithelial or sub-epithelial layers of the mucous membrane.

It is to be understood that any immune response arising from the devices and methods described herein is intended to:
only be induced by agents formulated to be immunogenic for a desired prevention or treatment of a disease or diseases; and
only prevent or treat such disease or diseases;

It is also to be understood that in delivering agents into and/or through tissues, the devices and methods preferably do not denature, cleave, break or otherwise damage the agent-, which may, among other things, generate an undesired immune response.

The tissue may contain or comprise of an epithelial membrane which may be a mucosal membrane or a cutaneous membrane. For example, the mucous membrane may form part of a subject's ocular conjunctiva, mouth, rectum or other parts of the gastro-intestinal system, genito-urinary and reproductive system including the vagina and uterus, the respiratory system including the nasal mucosa, larynx, pharynx, bronchi and lungs. The cutaneous membrane is skin. The tissue may also be the cornea, the tympanic membrane of the ear, teeth and nails.

The methods of embodiments of the invention described herein can also include one or more of the steps of:
loading the porated silicon material or reservoir with agent;
providing the porated silicon material or reservoir holding the agent;
bringing an agent transfer surface of the device into direct or indirect contact with said tissue; and
dispensing the agent from the device to the tissue surface, wherein the step of dispensing the agent preferably includes generating an acoustic signal to cause or facilitate transportation of the agent to the tissue-contacting surface.

By indirect contact it would be understood that a substance such as a gel may be interposed between the agent transfer surface of the device and the tissue in order to optimise transmission of the acoustic signal.

As would be understood by the skilled person, the delivery of agent to one selected layer may not be absolute. For example, the operational parameters of the device may be configured to deliver a sufficient amount of the agent and by ‘sufficient amount’ it would be understood to comprise an amount of riboflavin-5-phosphate-sodium in the anterior corneal stroma sufficient to, in the case of the treatment of keratoconus as an example, crosslink collagen using UV-A light. However some of the agent may also be delivered to Descemet's membrane. This small amount of ‘overflow’ is not contemplated to be delivery to both the corneal stroma and Descemet's membrane in accordance with the invention. Rather, if it is intended that a sufficient amount of agent be delivered to both the corneal stroma and Descemet's membrane the specific operational parameters of the agent applicator would need to be configured in order to specifically achieve delivery of a sufficient amount of the agent to all desired layers. Similarly, delivery of the agent through, for example, the corneal stroma and Descemet's membrane may result in some of the agent remaining in either or both of those layers; but for the purposes of the invention, a sufficient amount of agent will be delivered to the underlying tissue.

In some embodiments, delivery of an agent induces immunity against infections.

Delivery of Agents to the Eye and Treatment of Eye Conditions/Diseases

In some embodiments of the invention, the devices and methods may be used to deliver agent into the eye of a subject. The subject may, for example, be a human subject, a mammalian subject, or any other animal to which the device may effectively applied for the non-invasive delivery of an agent into the eye. Delivery of the agent into the eye may be facilitated by contacting the device (specifically the agent transfer surface of the device) with any one or more of the corneal epithelium, corneal limbus and/or the conjunctiva overlying the sclera. The device may be used to propagate acoustic waves facilitating delivery of the agent to the interior of the eye by transport of the agent through the device and delivery of the agent through the corneal epithelium, corneal limbus and/or the conjunctiva overlying the sclera.

In applications where the agent transfer surface of the device is applied to the corneal epithelium, the agent may be delivered through the epithelium and where after passing through the corneal endothelium, it can enter the aqueous humour in the anterior chamber. The agent may be circulated within the aqueous which circulates in the anterior chamber, through the pupil and around the lens into the posterior chamber. The agent in the posterior chamber aqueous may contact the vitreous humour and blood vessels of the ciliary body and uveal blood vessels in the pars plana and, from there, be distributed via the choroidal vasculature to the posterior segment of the eye.

In applications where the agent transfer surface of the device is applied to the corneal limbus or the conjunctiva overlying any part of the sclera, the agent may penetrate though the conjunctiva and sclera to the choroidal vasculature and be transported through it posteriorly via the choroid capillary network (the chorio-capillaris) to the retina that lies internal to it separated from the choriocapillaris by Bruch's Membrane and the Retinal Pigment Epithelium which is the principal barrier to the entry of agents to the retina.

It is noted that in the context of delivering therapeutically effective agents into the eye the devices and methods of the present invention provide advantages over conventional/known methods.

The corneal epithelium is the major barrier to the entry of drugs into the cornea and eye. In the case of smaller agents (e.g. 500 Dalton or less, soluble) some are capable of passive diffusion through the cornea and/or sclera in therapeutic amounts, where conventional non-invasive delivery methods use eye drops or wafers (which include polymers). The wafer is physically held between the surface of the eye and the internal surface of the eye lid in the superior or inferior “fornix” (a cul de sac formed between the eyelids and the eye whose surface is covered by conjunctiva) whereby the agent can slowly leech out the drug. Eye drops commonly need to be applied 4-5 times a day. There is a lack of compliance commonly associated with eye drops including after application not closing the eye gently or not closing the eye for the period as required which both result in a significant reduction in the amount of drugs delivered to the eye. Wafers require a medical professional to insert them and can cause discomfort and irritation to the eye and infections. Compliance with the use of wafers is very much less than compliance with eyedrops. These methods/devices rely of the production of tears and for the patient to blink each of which may significantly vary in the population. While some small molecule drugs through eye drops and wafers may reach the posterior segment tissue including retina, various clearance mechanisms in the eye and adsorption into tissue preceding the posterior segment result in the amount of drug delivered to this area being significantly lower than that delivered to the cornea and anterior segment. On this basis, only mild inflammatory or infectious diseases disease in the posterior segment are commonly treated (as possible) through eye drops or wafers. Severe infections and inflammation in the posterior segment require that these small molecules are delivered by intra-vitreal injection to achieve a concentration that is therapeutically effective in this area. Any acute vision threatening disease is not suitable for treatment with eyedrops or wafers. Large molecules, including immunoglobulins and immunoglobulin fragment molecules used to treat severe vision threatening diseases including Macular Degeneration, Diabetic Macular Edema and Retinal Vein Occlusions cannot be delivered by eyedrops and these conditions are treated currently by delivering drugs by intra-vitreal injections.

The devices and methods of the present invention partially or wholly alleviate some or all of these shortcomings.

In general, the amount of drug delivered to the cornea and/or sclera by the devices of the present invention is greater and more rapid than can be achieved by using eye drops or wafers inserted in the cul-de-sac. The devices of the present invention are also capable of delivering therapeutically significant amounts of drugs to the choroid and ultimately to the retina which cannot be achieved by drops or wafers inserted in the cul-de-sac.

The amount of drug delivered to the cornea and/or sclera by the devices of the present invention is also predictable as the devices are directly applied against the tissue and operated for a certain period, the amount of drug remaining in the device following treatment can be measured, and delivery of the drug is not reliant on patient compliance or a minimum production rate of tears or blink rate. The device overcomes the barrier effect of the corneal epithelium.

The devices of the present invention also need to be applied less frequently than eye drops and not continuously applied over an extended time period like wafers in the cul del sac. The devices of the present invention device may include software that can monitor usage and compliance.

In the case of larger agents (e.g. more than 500 Dalton and/or are insoluble) which are incapable of passive diffusion through the cornea and/or sclera in therapeutic effective amounts, to the best of the inventors' knowledge there is no conventional non-invasive delivery method or device currently available to deliver such agents into the eye. Conventional delivery of these drugs is through intraocular (into the vitreous cavity) injection. Other methods include surgically implantable slow release wafers into the interior of the eye.

The devices and methods of the present invention partially or wholly alleviate some or all of these shortcomings.

In addition to the advantage of being non-invasive, the amount of drug delivered into the eye that is required for treating a disease in the choroid or retina is potentially less than the amount required by conventional methods as following initial delivery through the conjunctiva/sclera, the drug is transported through the blood supply of the eye predominantly to the target tissue site and as is not diverted in relevant amounts away from the eye through various clearance mechanisms or absorbed or adsorbed into surrounding tissues that do not require treatment. A reduction in the amount of drug required delivered to the eye is advantageous as it reduces any side effects or risks associated with the drug including when it is cleared into the systemic circulation. For example “Avastin” (Bevacizumab) which is used to treat the wet form of age related macular degeneration can cause stroke through the drug entering the systemic circulation. Additionally, it may reduce the cost of both treatment and manufacture. Furthermore, conditions and diseases of the eye such as, for example, Wet Age Related Macular Degeneration, Diabetic Macular Edema (DME) and infectious and inflammatory diseases of the choroid create breaks in Bruch's Membrane and retinal pigment epithelium (RPE) which permits neo-vascular and leaky choroidal vessels to enter the retina causing local haemorrhage and subsequent scarring. The most effective therapeutic target tissue for therapeutic agents is the choroid since the natural blood flow may carry the agent to the region of the retina where its integrity has been breached by neo-vascular tissue originating from the choroid.

In some embodiments, the devices and methods of the present invention can be used to treat conditions/diseases of the eye in a subject by delivering a therapeutically effective amount of an agent to a tissue. The subject may be any one or more of an animal subject, a mammalian subject, or a human subject. The condition/disease may be any that benefits from the non-invasive delivery of a therapeutic amount of an agent to a target tissue/component within the eye.

The term “therapeutically effective amount” as used herein will be understood to mean an amount of a given agent or mixture of agents that when administered to a subject, will have the intended therapeutic effect. The intended or full therapeutic effect may occur by administration of one dose of the agent or agent mixture, or alternatively may occur after administration of a series of doses. Thus, a therapeutically effective amount may be administered in one or more doses/administrations. The precise therapeutically effective amount needed for a subject will depend upon factors including, for example, the subject's age, size, health, the nature, location, and extent of the condition/disease, and/or the therapeutics or combination of therapeutics selected for administration. The skilled worker can readily determine a therapeutically effective amount of a given agent by routine experimentation.

By way of non-limiting example, the devices and methods of the present invention can be used for the treatment of keratoconus and myopia.

Keratoconus is corneal condition where, due to laxity of the corneal stroma's collagenous infrastructure, the cornea gradually becomes an increasing conical shape that causes irregular astigmatism which when it progresses cannot be corrected by spectacles or soft contact lenses. Historic treatment has been using hard contact lenses and if these become unsuccessful, corneal transplant surgery must be performed in order to regain useful vision.

Corneal collagen cross-linking is currently being used as a treatment modality option to halt the progression of keratoconus by stiffening the collagen ultrastructure of the corneal stroma. Corneal collagen cross-linking (CXL) requires riboflavin-5 phosphate sodium to be within the corneal stroma. The barrier effect of the corneal epithelium retards the entry of riboflavin-5 phosphate sodium. The majority of current techniques surgically remove the corneal epithelium so as to enable the delivery of riboflavin-5 phosphate sodium to the stroma. Despite the barrier being removed, riboflavin-5 phosphate sodium containing drops must be applied every one or two minutes (usually for a period of 30 minutes) before the corneal stroma contains a sufficient concentration of riboflavin-5 phosphate sodium for the next stage of treatment being exposure to Ultraviolet Light-A can proceed. Following riboflavin-5 phosphate sodium absorption, the cornea is exposed to UV light (typically 365-370 μm) for a time period of 30 minutes to induce collagen crosslinking. After treatment, the cornea is at risk of infection because the epithelium has been removed and the resulting ulcer must heal by the epithelium growing back to cover the defect which takes several days. To limit the severe pain, a “bandage” soft contact lens is applied and antibiotic eyedrops are used at least 4 times a day until the ulcer is healed. The Ophthalmologist needs to review the patient to ensure that healing is complete and that no infection has developed.

The devices and methods of the present invention can be used to non-invasively deliver riboflavin-5 phosphate sodium salt (and/or substitutes known in the art such as glutaraldehyde [GD], grape seed extract [GSE], and/or genipin [GE]) into the cornea without removing or significantly weakening the corneal epithelium. For example, the agent transfer surface of a device according to the present invention can be contacted with the corneal epithelium. Acoustic waves propagated on and/or in the piezoelectric substrate of the device can be used to transport riboflavin-5 phosphate sodium through the device and deliver it through the corneal epithelium into the corneal stroma to a target depth. UV exposure can then be ordinarily used to induce collagen crosslinking. The corneal epithelium is not damaged by this non-invasive delivery, and the application can thus be repeated as frequently as necessary to achieve the desired outcome without subjecting a patient to the discomfort and risks associated with removing the corneal epithelium. The riboflavin-5 phosphate sodium can potentially be delivered to the stroma in 3 to 5 minutes which is 6 to 10 times faster that by the invasive conventional method currently used.

Current conventional treatments available for eliminating the dependence on spectacles and contact lenses for myopia (near sightedness) involve invasive excimer laser corneal surgery to effectively flatten the contour of the cornea by laser ablation of the stroma or removal of the lens and replacing it with a plastic intra-ocular lens.

Ortho-Keratology is non-invasive and requires that a patient wears a rigid contact lens (an Ortho-K lens) overnight which is removed on waking. The rigid lens flattens the cornea and temporarily reduces myopia so that the patient can function without a visual aid during the day. The effect of the flattening wears off during the ensuing hours and the rigid contact lens is inserted again the following evening before sleep. The Ortho-K hard contact lens can create corneal ulceration and sleep disturbance if it is uncomfortable. So as to retain the corneal shape created by wearing an Ortho-K hard contact lens for a long period, after removal of the Ortho-K lens, and after removal of the corneal epithelium, riboflavin-5 phosphate sodium/UV-A light collagen cross linking has been used by some investigators in an effort to retain the flattened corneal shape. It is known that collagen cross-linking continues for some hours after the UV-A light treatment phase is complete. It would be advantageous if the Ortho-K lens could be worn immediately after treatment but this cannot be done because there is an ulcer on the eye following the removal of the corneal epithelium. Due to the corneal epithelium being surgically removed or weakened to facilitate riboflavin-5-phosphate-sodium uptake, it is generally not possible to repeat the procedure for a number of months.

The devices and methods of the present invention can be used to non-invasively deliver riboflavin-5 phosphate sodium to the anterior corneal stroma without removal of the corneal epithelium. This can be used in a novel treatment of myopia that includes the following steps:
remove an Orth-K hard contact lens after wearing it overnight;
using the devices and methods of the present invention to non-invasively deliver riboflavin-5 phosphate sodium to the anterior corneal stroma without removal of the corneal epithelium;
performing the conventional UV-A light collagen cross linking procedure; and
immediately following the conventional UV-A light collagen cross linking procedure, reapplying the Orth-K hard contact lens on the cornea for at least two hours (as collagen cross linking continues for at least two hours following cessation of UV-A light exposure). This enables the corneal collagen cross-linking to continue to stiffen the cornea whilst it is being moulded to its ideal shape by the Ortho-K hard lens; and
following the above steps, normal use of the Orth-K hard contact lens can resume. The non-invasive nature of this treatment among other things enables the procedure to be repeated as often as clinically required to be effective.

In addition to the novel treatment for myopia above, the devices and methods of the present invention can be used to non-invasively deliver riboflavin-5 phosphate sodium to the anterior corneal stroma without removal of the corneal epithelium as a novel treatment of keratoconus that includes the following steps:
using the devices and methods of the present invention to non-invasively deliver riboflavin-5 phosphate sodium to the anterior corneal stroma without removal of the corneal epithelium; and.
performing the conventional UV-A light collagen cross linking procedure. This procedure can halt the progression or partly reverse keratoconus.

The non-invasive nature of this treatment among other things enables the procedure to be repeated as often as clinically required to be effective.

Some patients with keratoconus may benefit having an extra step as outlined above of immediately following the conventional UV-A light collagen cross linking procedure, applying an Orth-K hard contact lens on the cornea for at least two hours (as collagen cross linking continues for at least two hours following cessation of UV-A light exposure). This enables the corneal collagen cross-linking to continue to stiffen the cornea whilst it is being moulded to its ideal shape by the Ortho-K hard lens.

The devices and methods of the present invention can be used to non-invasively deliver agents (e.g. therapeutic agents in therapeutically-effective amounts) to the posterior segment of the eye.

For example, in some embodiments the devices and methods of the present invention may be used to deliver a therapeutically effective amount of the agent for treating a condition or disease upon delivery to the posterior segment of the eye by contacting the tissue transfer surface of the device with the corneal epithelium. In this manner, the agent may be delivered into and through the corneal epithelium, Bowman's membrane, corneal stroma and corneal endothelium into aqueous humour. The agent may then circulate within the aqueous humour through the pupil and around the lens into the posterior chamber where it may contact one or more of the vitreous humour, ciliary body blood vessels, uveal blood vessels in the pars plana, and be distributed via the choroidal vasculature to the posterior segment of the eye.

In other embodiments, the devices and methods of the present invention may be used to deliver a therapeutically effective amount of the agent for treating a condition or disease upon delivery to the posterior segment of the eye by contacting the tissue transfer surface of the device with the conjunctiva overlying the sclera. In this manner, the agent may be delivered into and through the conjunctiva overlying the sclera, and the sclera, and then enter the uveal tract of the eye where it can be distributed via the choroidal vasculature to the choroid and retina in the posterior segment of the eye.

Accordingly and again by way of non-limiting example the devices and methods of the present invention can be used for the treatment of conditions/diseases localised in or emanating from the posterior segment of the eye. Acoustic waves propagated on and/or in the piezoelectric substrate of the device can be used to transport drug through the device and through the conjunctiva overlying the sclera and sclera to the choroidal vasculature and be transported through it posteriorly via the choroid capillary network (the chorio-capillaris) to the retina. The devices and methods can be used to non-invasively deliver therapeutically effective amounts of an agent to a target tissue in the posterior segment of the eye (e.g. sclera, fovea, anterior hyaloid membrane, vitreous humor, retina, choroid, optic nerve, optic disc). Non-limiting examples of applicable conditions/diseases include Age Related Macular Degeneration, Diabetic Macular Edema (DME), infectious disease, and inflammatory diseases. Others include inherited diseases of the retina which may potentially be treated by the introduction of RNA and its sub types, DNA, and other biologics to such tissue.

For the purpose of this specification, the word “comprising” means “including but not limited to”, and the word “comprises” has a corresponding meaning. The terms “include”, “for example”, “non-limiting example”, “comprises” and “comprising” will each be understood to be non-exhaustive in relation to the subject matter following them.

The above embodiments have been described by way of example only and modifications are possible within the scope of the embodiments that follow.

The invention will now be described in more detail, by way of illustration only, with respect to the following example. The example is intended to serve to illustrate this invention, and should not be construed as limiting the generality of the disclosure of the description throughout this specification.

### EXAMPLES

The present invention will now be described with reference to specific Examples, which should not be construed as in any way limiting.

### Example One: Non-Invasive Drug Delivery Devices

TABLE 1
Aims and milestones

| | |
|---|---|
| Activity | Description |

Milestone 1

| | |
|---|---|
| Fabrication of pSi tips | Optimisation of critical fabrication |
| (Completed) | parameters for the preparation of pSi |
| | (nanoporated silicon) tip using electro- |
| | chemical etching. |
| Loading properties | Estimation of theoretical volume loading |
| (Completed) | capacity. |
| | Undertake test to measure loading |
| | characteristics using contact angle |
| | loading of the tip by applying ultrasound |
| | generated by the actuator within the |
| | MuPharma device. |
| Mechanical properties | Study of the mechanical properties of pSi |
| (Completed) | tips (loaded and unloaded). Trial to |
| | determine what pSi tip design is most |
| | able to withstand a range of ultrasonic |
| | frequencies and powers. |

Milestone 2

| | |
|---|---|
| Ejection properties | Ejection properties in air. This study aims |
| (Completed) | to understand the potential drug loss |
| | when tips are not in contact with tissue. |

Milestone 3

| | |
|---|---|
| Drug dispersal | Initial study to characterize drug dispersal |
| properties (benchtop) | into an agarose medium as a simulated |
| (Completed) | mucosal membrane using a fluorescently |
| | tagged antibody. This allows for simple |
| | observation and measurement. |
| | Benchtop/ex-vivo studies into pig cadaver |
| | eyes demonstrating that the MuPharma |
| | device (incorporating the pSi tips) can |
| | deliver fluorescently tagged drug |
| | substitutes into biological tissue. |

Milestone 4

| | |
|---|---|
| Avastin delivery study | Live rabbit study to determine whether |
| (In Vivo) | the MuPharma device (incorporating the |
| (Completed/) | pSi substrate) can non-invasively deliver |
| | radiolabelled Avastin to the posterior |
| | segment of the eye as assessed by real- |
| | time Positron Emission Tomography |
| | imaging (PET), Magnetic Resonance |
| | Imaging (MRI) and histology. |

1. Optimisation of Critical Parameters of pSi Tip Fabrication

pSi films were fabricated by electrochemical dissolution of a highly doped, (100)-oriented, boron-doped p-type crystalline silicon wafer (0.0005-0.001 Ω·cm resistivity) using an electrolyte mixture of 48 wt. % hydrofluoric acid (HF) in absolute ethanol (99.9%) in a volumetric ratio of 3:1, respectively. At first, the pSi sacrificial layer was generated by anodisation using the current density values listed in Table 1 for 30 s and dissolved by alkaline dissolution of sodium hydroxide (NaOH, 1M) to avoid the formation of a parasitic layer (with pores of few nm in diameter) restricting the diffusion of the bioanalyte inside the pSi layer. The pSi layers were then etched by applying the electrical current densities and etching times listed in Table 1 to determine the optimum fabrication conditions for thick layers 350 μm) with pore dimensions that allow the diffusion of a fluorescently-tagged antibody (MW 150 kDa). In addition to the latter features, superior structural properties of these films are highly desirable since the pSi layers will serve as a nanoscale reservoir (carrier material) for ultrasound-mediated drug delivery applications and should be structurally robust enough to withstand the application of ultrasound frequencies 50 kHz), without the collapse of the thick porous scaffold.

TABLE 2
Experimental parameters used for the preparation of the pSi tips.

| Current density | Etching | |
|---|---|---|
| (mA · cm⁻²) | time (min) | Results |
| 233 | 30 | Collapse of the structure after fabrication |
| | | (during drying process) |
| 195 | 30 | Collapse of the structure after fabrication |
| | | (during drying process) |
| 162 | 30 | Collapse of the structure after fabrication |
| | | (during drying process) |
| 152 | 30 | Collapse of the structure after fabrication |
| | | (during drying process) |
| 130 | 30 | Collapse of the structure after fabrication |
| | | (during drying process) |
| 100 | 30 | Collapse of the structure after fabrication |
| | | (during drying process) |
| 83-55 | 150 | Detachment of the thick open-ended pSi |
| | | membrane after the antibody-loaded tip |
| | | was allowed to dry in air. |
| 50 | 240 | pSi tips exhibiting optimal structural |
| | | properties capable of resisting |
| | | ultrasound and evaporation in air of the |
| | | loading solution. |

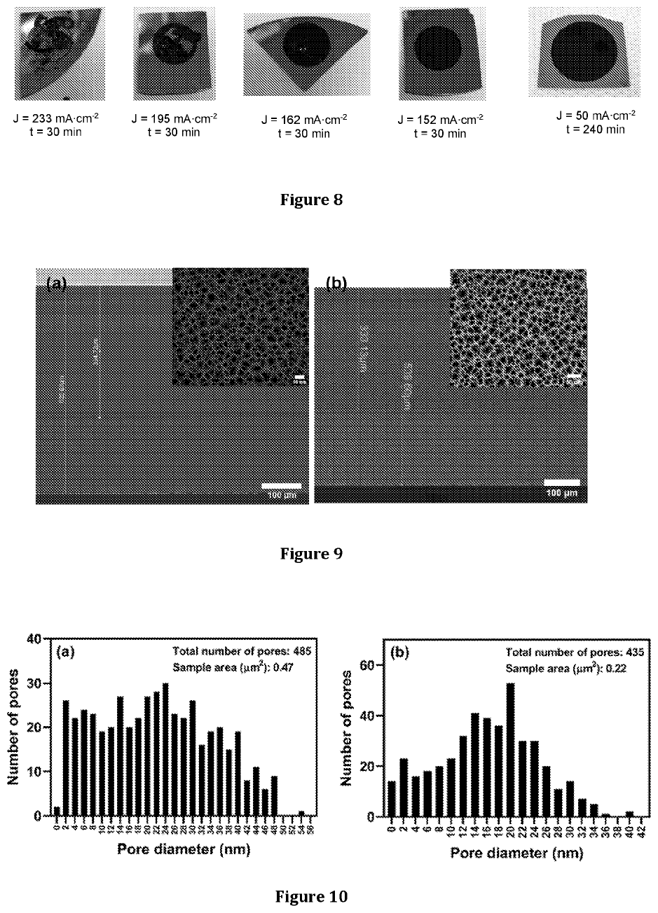

After testing the different fabrication conditions, we found that a current density value of 50 mA·cm⁻² and an etching time of 14,400 s (240 min) resulted in pSi films with a thickness of ˜350 μm displaying superior structural properties (FIG. 8). These pSi tips were able to resist the evaporation (in air) of aqueous-based loading solutions and the application of a range of ultrasonic frequencies (40-55 kHz) and voltages (30-150 V) using the MuPharma device without evidence of structural damage. On the other hand, pSi tips obtained using current densities between 83 mA·cm⁻² and 55 mA·cm⁻² could withstand evaporation of the loading solution if a small volumetric percentage of a surfactant agent (i.e., ethanol) is added to reduce the surface tension of water exerted on the pore walls, thus avoiding the detachment of the thick porous layer from the crystalline silicon substrate.

2. Morphological Characterisation of the pSi Tips

Morphological characterisation of as-prepared pSi films (i.e., pore diameter and thickness) was performed using scanning electron microscopy (SEM). FIG. 9 (a) and (b) show typical outcomes of SEM analysis performed on the cross-section of two pSi layers prepared using current density values of 83 mA·cm⁻² for 150 min and 50 mA·cm⁻² for 240 min applied to a 525 μm thick silicon wafer, respectively. From these images, it can be inferred that the resulting etching depths feature a similar column-like morphology with a thickness of ˜334 μm and ˜333 μm, respectively. The pore depths are continuous from top to bottom throughout the thickness of the pSi layer, indicating that the pores act as individual nanoscale channels (wells) with negligible interconnection between adjacent channels.

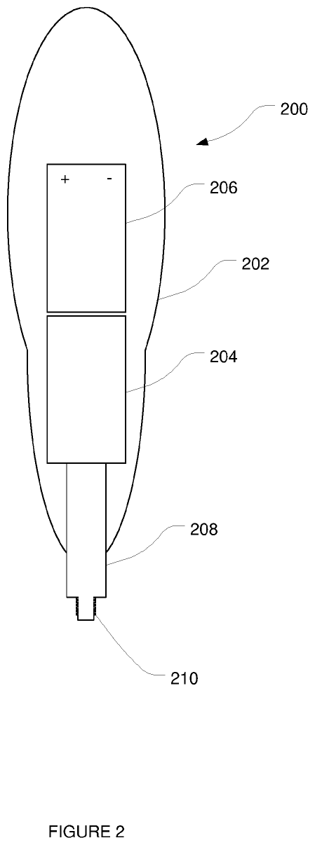

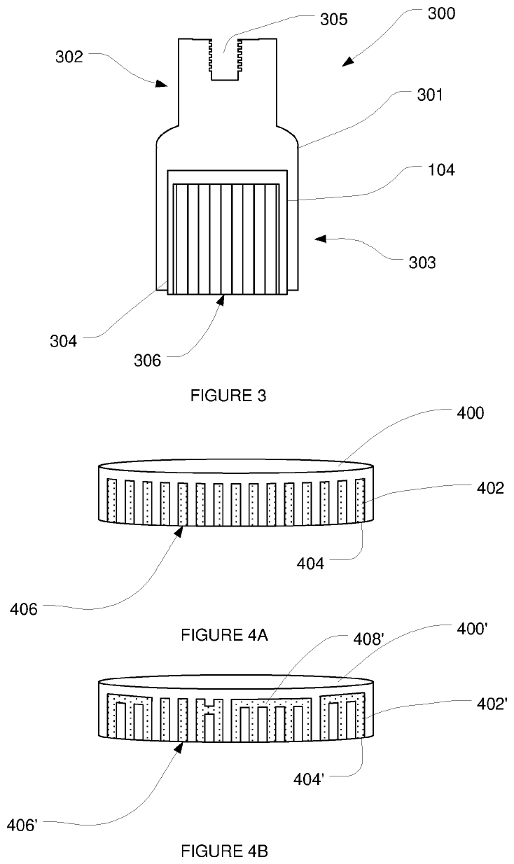

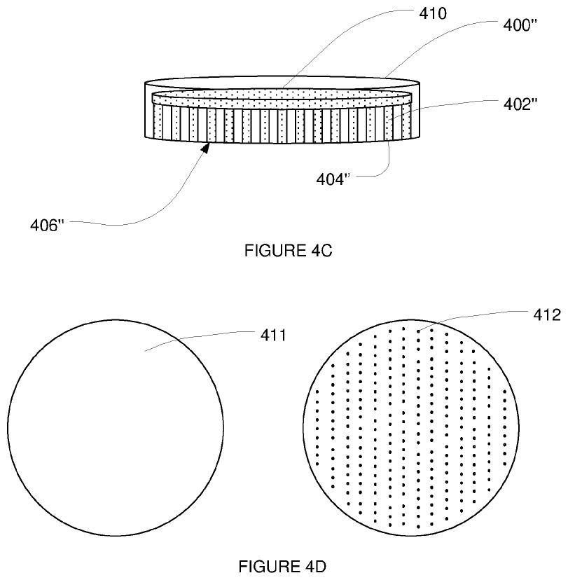

Characteristic pore size distribution and morphology of these pSi tips can be observed in the top-view SEM micrograph presented as insets in FIGS. 2 (a) and (b), respectively. Complete surface opening of these nanopores, which are large enough to allow effective infiltration of biomolecules with characteristic dimensions of a few nanometers, was achieved by the sacrificial layer dissolution prior to etching of the pSi layer. Knowledge of the pore size distribution in a given pSi film is essential to assess the optimal diffusion of the analyte throughout its depth. Pore size histograms obtained using the top-view SEM images are shown in FIGS. 10 (a) and (b), respectively. Thresholding of the grey-scale top-view SEM micrographs of pSi in ImageJ software yields binary images that allow for straightforward differentiation between the pores and the silicon pore wall framework. After noise removal using mathematical morphological openings and closings on the threshold image, pore areas in pixel count were extracted using the Particle Analysis method of ImageJ. The pore areas were then expressed in nm² by application of a conversion factor based on the ratio of the SEM scale bar pixel count to the corresponding length in nm. Finally, effective pore diameters were calculated from the extracted pore areas. FIG. 10 (a) depicts a pore size distribution between 0.8 to 55 nm (σ=12.69 nm) with a mean pore size of 21.9 nm and a total number of 485 nanopores contained in an area of 0.738×0.638 μm² of the pSi surface (i.e., the observable pSi area in the top-view SEM inset of FIG. 9(a)). FIG. 10 (b) presents a pore size distribution ranging from 0.55 to 40.2 nm (σ=8.34 nm) with a mean pore size of 16.2 nm and a total number of 435 nanopores contained in an area of 0.502×0.433 μm² of the etched silicon surface (i.e., observable pSi area in the SEM inset of FIG. 9(b)).

3. Theoretical Volume Loading Capacity

Theoretical volume loading capacity, porosity, and thickness were calculated based on gravimetric analysis of the pSi layer. This is a test that takes advantage of the fact that freshly etched porous silicon dissolves rapidly in aqueous solutions having a basic pH. The gravimetric method is based upon the definition of porosity P as the ratio of the volume of the pores to the total apparent volume of the film:

$$P = \frac{{V}_{pores}}{{V}_{total}} \qquad ( 1 )$$

The gravimetric measurement is performed by weighing the sample before etch (m₁), after etch (m₂), and finally after chemical dissolution of the porous layer (m₃). The volume of the pores is assumed to be equal to the volume of silicon that is removed during the electrochemical etch, which is related to the mass of the wafer before (m₁) and after (m₂) etching:

$${V}_{pores} = \frac{{m}_{1} - {m}_{2}}{{δ}_{Si}} \qquad ( 2 )$$

Where δ_{Si} is the density of elemental silicon. The total apparent volume of the porous film (including voids) can be determined from the mass of the wafer before etching (m₁) and the mass of the wafer after the porous layer has been removed (m₃):²

$${V}_{total} = \frac{{m}_{1} - {m}_{3}}{{δ}_{Si}} \qquad ( 3 )$$

Applying equations (1) and (2) to (3) yields to equation (4]):²

$$P = \frac{{m}_{1} - {m}_{3}}{{m}_{1} - {m}_{2}} \qquad ( 4 )$$

The measurement also yields the thickness of the porous layer (W), which is dependent on the density of the material and the planar area of the wafer that was exposed to the etching solution:²

$$W = \frac{{m}_{1} - {m}_{3}}{A {δ}_{Si}} \qquad ( 5 )$$

where A is the wafer area exposed to HF during the electrochemical etch. The value of δ_{Si} can be taken as 2.33 g ml⁻¹.

Table 3 shows the theoretical calculations for the porosity, thickness and loading capacity using the gravimetric analysis for a pSi tip (area of ˜25 mm²) prepared using a current density of 83 mA·cm⁻² for 150 min (FIG. 9 (a)). Our calculated theoretical value of 357 μm in thickness of the film is in close agreement with the value of 334 μm obtained using SEM analysis. Moreover, we determined that there was a porosity value close to 79% by volume and a theoretical loading capacity of ˜7 μL.

TABLE 3
Theoretical porosity, thickness and loading
capacity determined based on gravimetric analysis.

| | m₁ | m₂ | m₃ |
|---|---|---|---|
| Mass (g) | 0.6543 | 0.57462 | 0.55434 |

| | |
|---|---|
| | Porosity |
| $P = \frac{{m}_{1} - {m}_{2}}{{m}_{1} - {m}_{3}}$ | 0.79 |
| | Thickness (μm) |
| $\begin{matrix}W = \frac{{m}_{1} - {m}_{3}}{A {δ}_{Si}} \\ A = 1.2 {cm}^{2} \\ {δ}_{Si} = 2.33 g / {cm}^{3}\end{matrix}$ | 357 |
| | Spatial volume (μL) |
| V = X * Y * Z | 8.9 |
| X = Y = 5 mm | |
| Z = 0.357 mm | |
| | Loading capacity (μL) |
| Vᵣₑₐₗ = V * P | 7.031 |

Additionally, using the spectroscopic liquid method infiltration (SLIM)², a resulting porosity of around 60-65% was estimated for the pSi tip prepared using a current density of 50 mA·cm⁻² for 240 min.

4. Loading Capacity Tests

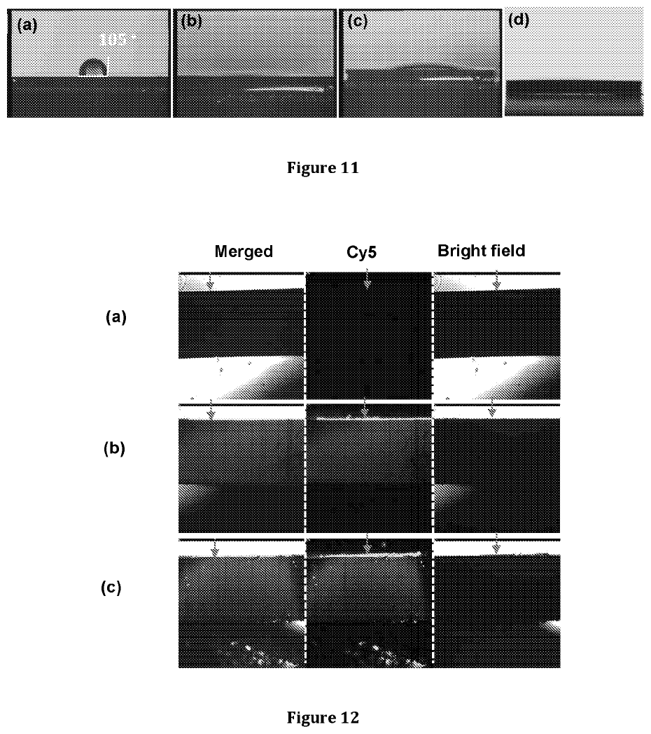

In order to study the loading capacity of the pSi tips, water-based contact angle measurements were carried out. The hydride-terminated surface of freshly etched pSi films displays hydrophobic properties impeding the penetration of aqueous-based solutions into the nanochannels. A conventional chemical strategy to overcome this issue is thermal oxidation of pSi surfaces in air. On this latter point, a two-step oxidation approach was adopted for our nanostructured surfaces: (1) thermal oxidation in air of the pSi tip (400° C., 60 min) followed by (2) ozone oxidation for 30 min (O₂ flow rate of 0.5 L min⁻¹). FIG. 11 shows the contact angle measurements performed on the surfaces of a (a) as-prepared pSi tip, (b) thermally oxidized pSi tip, and (c) and (d) thermally and ozone oxidized pSi tips. For surfaces (a) and (b) after dropping 1 μL of ultrapure water, a photograph was immediately taken. FIG. 11 (a) confirms the hydrophobic characteristics of the as-prepared pSi surface (contact angle of 105°), whereas FIG. 11 (b) shows a hydrophilic surface after thermal oxidation of the pSi tip. Furthermore, for the surface in FIG. 11 (c), a volume of 7 μL (i.e., theoretical loading capacity) of phosphate-buffered saline (PBS) solution was dropped cast on the pSi tip oxidized using the two-step oxidation approach. The formation of a meniscus on the surface implies partial spreading of the test solution. However, when ethanol (2.5%, v/v) was added to the buffer solution (to improve infiltration), the formation of a negligible meniscus on the pSi surface was observed (FIG. 11 (d)). The latter result confirms a homogeneous infiltration of the test solution into the porous matrix. pSi tips used in all the experimental work herein were carefully cut in small pieces with a dimension of 5 mm×5 mm (area of 25 mm²).

In summary, the optimization of critical fabrication parameters for the preparation of pSi tips, as well as the study and characterization of the loading properties of these tips, were completed (Milestone 1).

5. Structural Stability (Mechanical) and Ejection Properties

Superior structural (mechanical) properties of these films are highly desirable, given that pSi tips will serve as a nanoscale reservoir for ultrasound mediated drug delivery applications. Thereby, pSi tips should be structurally robust enough to resist the application of ultrasound frequencies 50 kHz) without the collapse of the porous scaffold. Additionally, the ejection properties of these porous substrates (loaded and unloaded) were observed through an optical microscope and in a slow-motion clip (960 fps), which was recorded using a smartphone secured to one of the eyepieces of the microscope. In these videos, one can observe the pSi tip attached to the MuPharma ultrasonic pen. The tip comprises a brown-like porous layer with ˜334 μm in thickness on top of a crystalline silicon substrate. Ultrasound frequency of transducer (connected to pSi substrate) was 50 kHz (sinusoidal waveform, Vₚₚ=30 V). The first video (attached file ‘video_pSi_1’) recorded the effect of ultrasound applied to an unloaded pSi tip attached to the transducer. From this video, we can observe that the tip is able to resist the maximum ultrasonic frequency we selected for the study using the MuPharma device without collapsing the porous scaffold. Furthermore, if carefully observed, one can notice the oscillation of this system due to the sinusoidal frequency applied. A second video clip (attached file ‘video_pSi_2’) recorded the effect of the ultrasound on the same pSi tip loaded with a methylene blue aqueous solution (˜7 μL). In this video clip, one can observe the volume of the test solution dispensed on the surface of the tip. Slow-motion mode allows appreciating the diffusion of the solution into the porous matrix with the aid of the ultrasound application. The porous structure of the tip was preserved and undamaged upon loading and sonication. By building on these results, we are confident to say that the mechanical properties of our pSi tips are robust enough to endure loading and sonication procedures.

On the other hand, the ejection properties of the tested tips are difficult to observe in the time-lapse video clips due to the limiting factor of the resolution of the instruments that were used. However, we hypothesized that in order to promote an effective diffusion of the analyte hosted within the porous matrix (i.e., from a region of high concentration to an area of low concentration), contact against the receiving medium (i.e., mucosal membrane, tissue) is critical. The sonophoretic effect of ultrasound mediated delivery would enhance the passage of an analyte. With these results in mind, Milestone 2 was completed.

6. Antibody Diffusion Test

To further characterize the diffusion properties of the pSi tips. A drug substituent consisting of a fluorescently labelled antibody (Ab) in buffer, displaying the same molecular weight (150 kDa) of Avastin (Bevacizumab), was prepared. The substituent Ab is tagged with a fluorescent moiety Cy5 (Cyanine-5), which has excitation/emission wavelengths of 633/647 nm, respectively. Images showing the diffusion of the labelled Ab were obtained using confocal laser scanning fluorescence microscopy (CLSFM). A set of three optical images containing: (a) a bright-field mode image, (b) a Cy5 channel fluorescence image, and (c) the resulting merged images of (a) and (b), were collected for each pSi membrane under study. The pSi membranes (˜334 μm in thickness) were carefully detached from the crystalline silicon substrate after oxidation step (thermally and ozone treated) and drop cast with the test Ab solution (˜7 μL). Afterward, the membranes were allowed to dry off in air (˜15 min) and a new fresh cross-section sample of the membrane was obtained prior to CLSFM imaging analysis. FIG. 12 (a) shows the pSi membrane referred to as control after diffusion of the blank solution (PBS buffer). No emission or fluorescence background was observed when the sample was irradiated with the Cy5 laser frequency (excitation wavelength of 633 nm). pSi membranes in FIGS. 12 (b) and (c) were tested using a buffer solution containing the fluorescently tagged Ab (0.19 mg/mL, 1.26 μM). The resulting confocal images verified the characteristic emission of the Cy5 moiety attached to the antibody (emission wavelength of 647 nm). These results demonstrate the diffusion of the drug substituent throughout the depth of the pSi nanochannels. Nonetheless, it is important to note that at the top surface of these membranes, a strong emission of the Ab was clearly noted. We attribute this strong fluorescence to the pre-concentration effect of the Ab due to the formation of a meniscus after the Ab solution was drop cast on the surface of the membrane. When the solution was allowed to evaporate (in air), the Ab contained in solution of the formed meniscus was able to concentrate on the membrane's surface originating a strong emission. For these experiments, the Ab solution did not contain any surfactant agent such as ethanol to improve the wettability of the nanostructure, leading to the formation of the aforementioned meniscus. In all the CLSFM images presented in FIG. 12, the top surface of the tested pSi membranes is indicated by the red arrow.

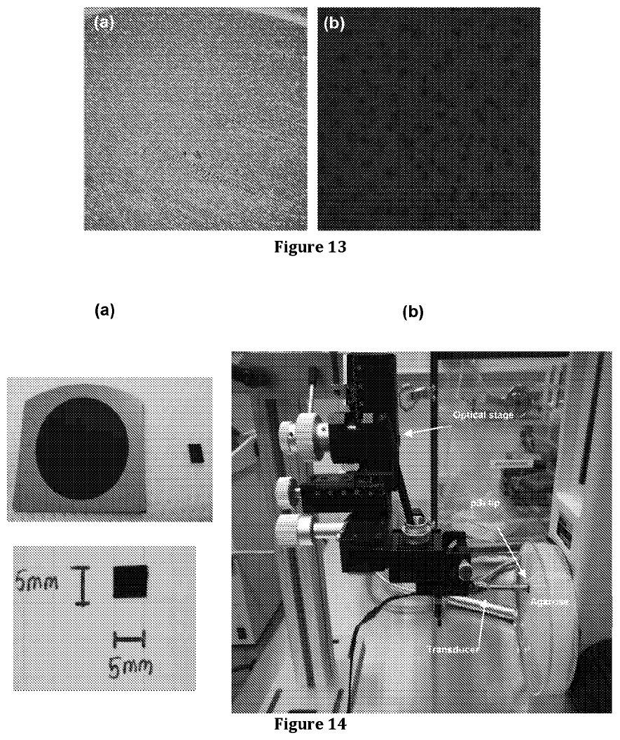

Furthermore, FIG. 13 (a) shows a confocal image showing the strong emission of the Ab at the top surface of one of the pSi membranes presented in FIG. 12. As already discussed above, the strong fluorescence is due to the pre-concentration effect of the Ab contained in the solution meniscus formed at the membrane top surface after spotting and allowing to dry in air. FIG. 13 (b) shows the fluorescence from the open-ended bottom surface of the pSi membrane after adjustments in the laser power during confocal analysis. The observation of this fluorescence confirms the extent of the Ab diffusion throughout the etched depths.

7. Ultrasound Mediated FITC Delivery into Agarose

pSi tips were tested using ultrasound mediated fluorescein isothiocyanate, (FITC, MW 389 Da) delivery into agarose hydrogel. FITC is a strong fluorescent dye with emission/excitation wavelengths of 485/525 nm, respectively. A solution of 0.1 mM FITC prepared in PBS containing DMSO (10%, v/v) was used as the test solution, whereas Agarose hydrogels (1.2% w/v) were used as the receiving medium for the ultrasound mediated FITC delivery experiments. pSi tips (5 mm×5 mm) were diced from a pSi layer with a thickness of ˜333 μm etched using a current density of 50 mA cm⁻² for 240 min for all the experiments (FIG. 14(a)). A photograph of the experimental setup for ultrasound mediated delivery experiments is presented in FIG. 14 (b). A multi-axis optical stage was used to finely control the tip displacement (x-, y- and z-axis), Mμ Pharma ultrasound transducer was secured in the stage and the pSi tip was then mounted on top of the titanium reservoir attached to the ultrasound traducer. It is important to consider a possible ‘dumping effect’ caused by the gap in the interface between the top of the transducer horn and the base of the pSi tip. The pSi tip needs to be firmly secured and air must be excluded to achieve optimum performance. In order to overcome the problem of the gap of air, carbon tape was applied as an intermediate, but this somewhat dampens ultrasonic energy transmission which significantly reduces the delivery performance of the pSi tip.

The ultrasound transducer was operated by applying the voltage and frequency values established for the solid titanium tip (conventional tip) used in previous MuPharma studies, i.e., peak to peak voltage of 30 V, and a sinusoidal waveform with a frequency of 50 kHz. An ultraviolet (UV) lamp was used as the excitation source for FITC. Loading of FITC into the pSi tips was performed as follows, a volume of ˜7 μL was drop cast on top of the pSi surface (thermally and ozone oxidized) and allowed to diffuse into the porous matrix (˜333 μm thick) by operating the ultrasonic transducer for 10 s (×3 times) for a total time of 1 min (tip and transducer positioned vertically in the multi-axis stage) to aid loading into the substrate. Then, the transducer carrying the loaded tip was positioned horizontally to allow pressing of the pSi tip into the agarose hydrogel surface (see FIG. 14 (b)). The tip was then imprinted against the agarose hydrogel by controlling the displacement (˜2 mm) using one of the millimetric optical stage axes. FITC delivery into agarose was tested with and without ultrasound application. Afterward, the imprinted regions were carefully extracted from the main agarose framework contained in the Petri dish in the form of cubes with dimensions of ˜1×1 cm².

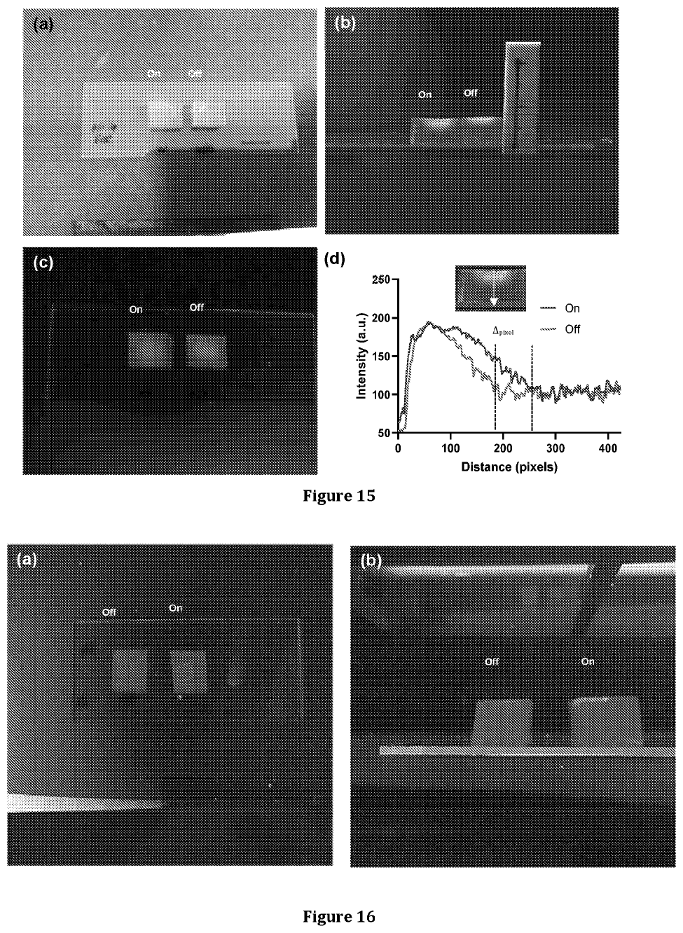

FIGS. 15 (a) and (c) show a top-view photograph of the agarose hydrogel cubes tested with the pSi substrate with and without ultrasound under ambient- and UV-light conditions, respectively. An ultrasound sinusoidal waveform with a frequency of 50 kHz was applied for 30 s for the ‘On’ labelled agarose cube sample, whereas no ultrasound was applied for the control sample (i.e., “Off” labelled agarose cube). A cross-section photograph of these agarose hydrogel cubes under UV light is shown in FIG. 15 (b). The extent of FITC diffusion into agarose hydrogel is more evident in the sample tested with ultrasound (On′ labelled) in comparison to the agarose cube tested without it (Off labelled), as depicted in FIG. 15 (b). The pSi tip with or without ultrasound was applied to the agar surface for a total time of 30 s; however, extended application times could potentially result in an improved ultrasound mediated delivery of FITC into the receiving media.

Additionally, to quantitatively obtain an estimation of the FITC diffusion extent into the agarose samples, a transverse intensity profile was measured under UV light along each tested agarose cube using ImageJ software. Transverse profile was measured (in pixel counts) from top to bottom of each agarose cube (inset of FIG. 15 (d)), producing a proportional intensity value of the image along the measured axis as shown in the profiles of FIG. 15 (d). The penetration depth of FITC in the sample tested with ultrasound resulted in a 65 pixels deeper diffusion (i.e., Δₚᵢₓₑₗ=65) as compared to the sample tested without ultrasound (i.e., control sample) before both intensities profiles reach the background intensity level.

8. Ultrasound Mediated Antibody Delivery into Agarose

Delivery properties of the pSi tips loaded with a FITC-tagged Ab (Bevacizumab, MW 150 kDa) solution were also conducted to investigate the effect of ultrasound on Ab delivery into agarose. In a typical experiment, 7 μL of a PBS buffer containing 0.2 mg/mL (1.3 μM) of a FITC labelled Ab were loaded into the pSi tips as described in the previous section. FIG. 16 (a) shows a top-view photograph of the agarose cubes tested with the Ab solution with and without ultrasound under UV light. An observable fluorescence emission can be noted from the agarose tested with ultrasound in comparison to the negligible emission from the surface of the agarose tested without the ultrasound application. These results are further supported when we inspected the cross-section photograph of these agarose cubes in FIG. 16 (b). From this image, it can be presumed that the application of ultrasound allows a relatively superior diffusion of the Ab into the receiving medium when compared to the delivery approach without the ultrasound application. Ultrasound mediated drug delivery approach hence seems to offer an advantage by facilitating the permeation of a specific drug into the tissue under study.

9. Ultrasound Mediated Antibody Delivery into Enucleated Pig Eyes.

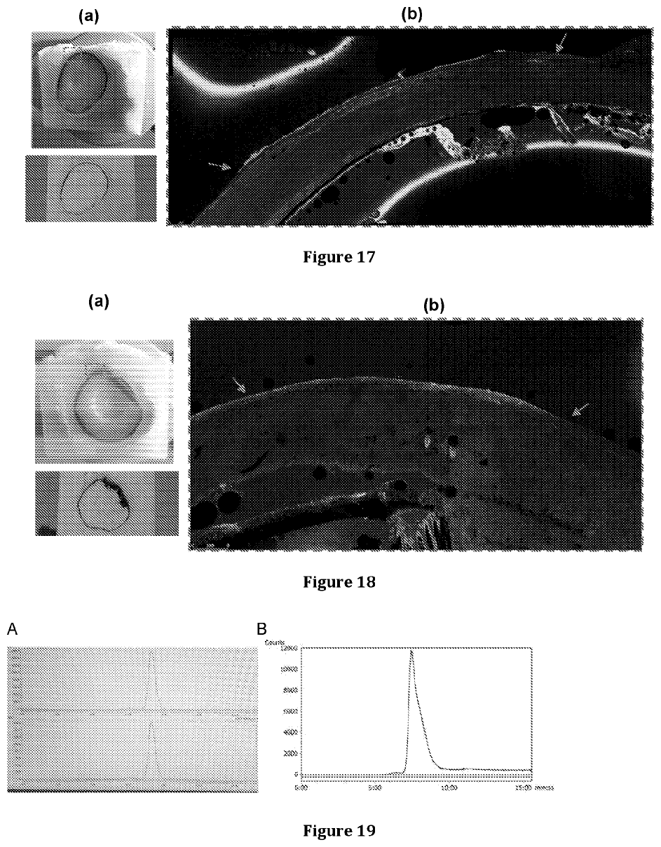

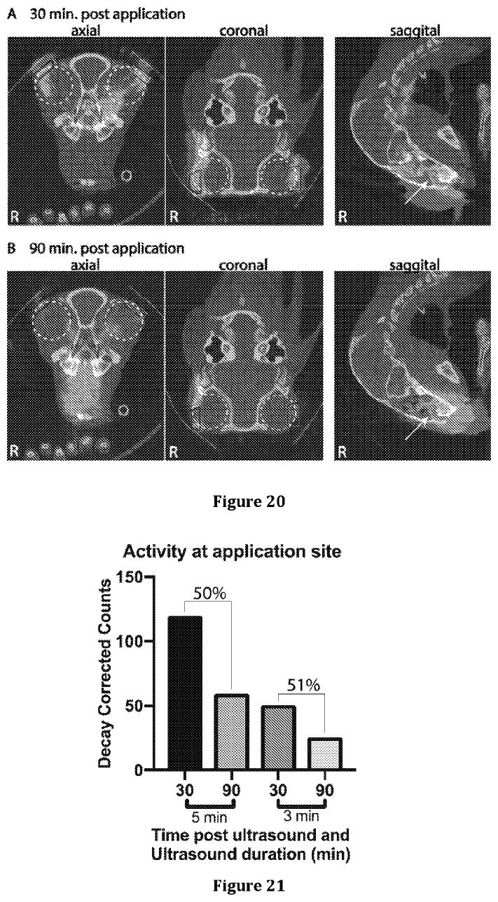

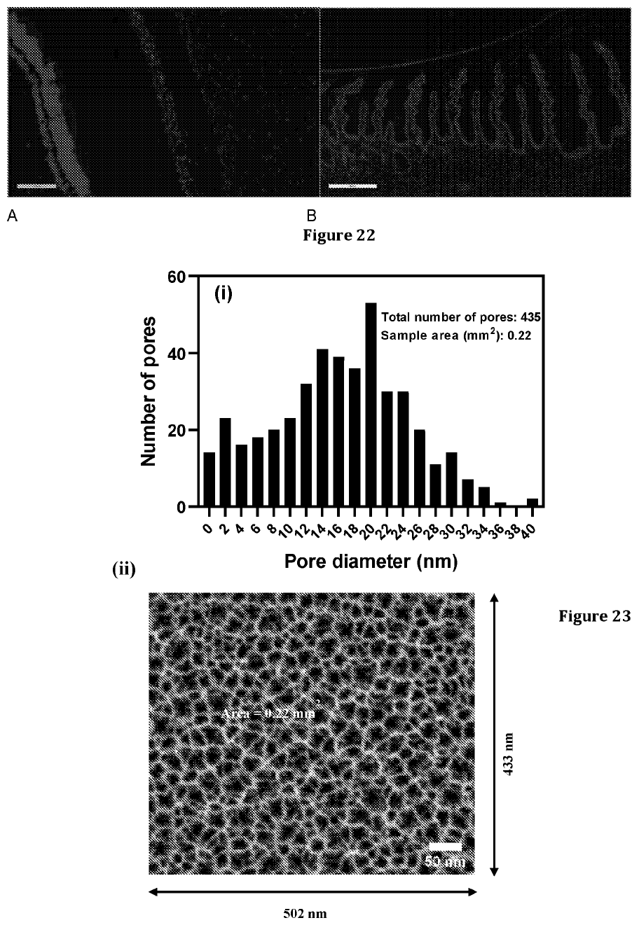

Ex-vivo experiments were performed in cadaver pig eyes. Pig eyes were enucleated approximately 3 h before the experiment. Eyes were transported in a cooled container at approximately 3° C. and were, at all times, regularly lubricated with SYSTANE® Lubricant Eye Gel. The drug substituent solution contained a fluorescently tagged antibody (Bevacizumab, MW 150 kDa) at a concentration of 0.19 mg/mL (˜1.3 μM). Cy5 moiety was bioconjugated to the antibody as the fluorescence label having excitation/emission wavelengths of 633/647 nm, respectively. Cadaver eyes tend to have reduced intra-ocular pressures, so normal saline solution was injected into the vitreous in the posterior segment of the eye in order to emulate a normal live intra-ocular pressure prior to the experiments. pSi tip was then mounted to the frequency ultrasound transducer and loaded as aforementioned in section 6. For each application to the eye, pSi tips were loaded with 10 μL of the Ab test solution. Two eyes were tested, one using ultrasound assisted delivery and the other by pressing the pSi tip against the eye without ultrasound. Ultrasound frequency of transducer (connected to pSi substrate) was 50 kHz, applied for 300 s to the scleral tissue of each eye. After the test, the eye was immediately submerged in a PBS (pH 7.4) solution containing 4% paraformaldehyde (PFA) at 4° C. for 24 h for sample preparation and cryopreservation. Afterward, the fixed eye was placed in 15% sucrose solution in PBS until the eye sunk (6-12 h) and then in 30% sucrose in PBS overnight. Then, the eyeball was embedded in OCT (Optical Cutting Temperature) media, avoiding bubble formation around the specimen and immediately frozen and stored at −80° C. (FIG. 17 (a), upper image). Finally, cryosections samples of the scleral tissue where the pSi tip was applied were prepared on a glass coverslip using mounting media prior to fluorescence microscopy imaging (FIG. 17 (a), bottom image). Red rectangles in the photographs of FIG. 17 (a) and FIG. 18 (a) enclose the region of interest (ROI) on the scleral tissue where the pSi tip was applied with and without ultrasound, respectively. Fluorescence analysis was performed using Fluorescence Lifetime Imaging Microscopy (FLIM). Briefly, two different excitation/emission channels (for FITC and Cy5) were used to visualize the light-emission properties of the Cy5-tagged Ab. Different fluorescence images were scanned around the perimeter of the ROI in the biological samples (×10 objective); once the ROI was properly scanned, the resulting tile images were merged to construct the fluorescence image shown in FIG. 17 (b) and FIG. 17 (b), respectively. From FIG. 17 (a), a strong magenta emission from the Cy5 moiety is plausibly observed across the region where the pSi tip was applied with ultrasound application. On the other hand, the cadaver pig eye tested in the absence of ultrasound showed negligible Cy5 emission, as confirmed by the fluorescence image of FIG. 18 (b). We attribute that the emission noticed in the eye treated with ultrasound is due to the effective permeation of the Ab into the scleral tissue. On the contrary, the eye tested in the absence of ultrasound showed an insignificant uptake of the Ab mainly because the majority of the delivered product remained on the external surface of the tissue, increasing its propensity to be washed in the subsequent steps for sample preparation. By building on the results presented in sections 6 to 9, Milestone 3 was successfully completed.

10. Pore Availability for Admission of Avastin

Avastin (MW 150 kDa) displays a hydrodynamic diameter of approximately 9.16 nm. Therefore, taking into account factors including randomised pore geometry shapes, admission of Avastin into the porous scaffold is expected for pores featuring a diameter above 15 nm. The method followed to estimate the number of pores fulfilling this requirement is presented in FIG. 23. The pore histogram presented in FIG. 23 (i) shows the total number of pores (e.g., 435 pores) in an area of 0.220 mm² enclosed in the observable topography of the top-view SEM image (FIG. 23 (ii)) of the porous surface. A relative percentage of 48% (e.g., 209 pores) from the totality of these feature the dimension required for Avastin diffusion into the nanostructure (Table A2).

TABLE 4
Pore distribution organised as a function of different diameters.

| Pore diameter | |
|---|---|
| (nm) | Frequency |
| 0 | 14 |
| 2 | 23 |
| 4 | 16 |
| 6 | 18 |
| 8 | 20 |
| 10 | 23 |
| 12 | 32 |
| 14 | 41 |
| 16 | 39 |
| 18 | 36 |
| 20 | 53 |
| 22 | 30 |
| 24 | 30 |
| 26 | 20 |
| 28 | 11 |
| 30 | 14 |
| 32 | 7 |
| 34 | 5 |
| 36 | 1 |
| 38 | 0 |
| 40 | 2 |

### Example Two: Optimization and Fabrication of Porous Silicon Tips

This Example describes the fabrication and optimisation of nanoporous tips based on porous silicon (pSi) by means of electrochemical dissolution of heavily doped crystalline silicon wafers. These tips have the advantage (among other things) of a larger surface area, which enables superior capillary action for hosting/loading drugs within the porous substrate to serve as carrier materials for ultrasound-mediated drug delivery applications.

### 1. Background

A first report (MDPP) describing the fabrication, characterisation, and physical properties of a set of nanotips was presented. A heavily doped p-type silicon wafer with a resistivity range between 0.55 to 1.0 mΩcm was used as the starting material in that study. The theoretical loading capacity of such tips was estimated to be of ˜7.0 μL. The surfaces were fabricated applying a current density of 83 mA·cm⁻² for 150 min. However, when these nanostructures were tested, detachment of the porous film from the underlying bulk silicon substrate was observed. The separation of the porous film from the crystalline substrate was attributed to the (mechanical) stress induced upon air-drying of the solution loaded within the nanostructure. Crystalline silicon is a strong but brittle material. When silicon is turned into pSi, the porosity lowers its hardness, stiffness, and fracture strength. If the nanostructure becomes weak, it can not survive standard material processing techniques such as air-drying and post thermal oxidation (1). In addition, when pSi is fabricated via any route that uses liquid, it requires careful drying. Drying stresses due to surface tension of the liquid admitted into pores are primarily responsible for the cracking, flake-off, or collapse of the porous scaffold. Different current densities were then tested to manufacture robust and thick porous layers displaying improved structural properties capable of surviving post-anodisation procedures such as thermal oxidation, dicing of the sample, air-drying, and the application of ultrasound frequencies. A current density of 50 mA·cm⁻² (applied for 240 min) was found to produce such robust and stable nanostructures (as stated in the initial study). However, the decrease in the sample's porosity reduced the loading capacity from 7.0 to 3.8 μL.

The loading capacity of pSi sample is a parameter directly related to the porosity of the film. In the initial report, gravimetric analysis was employed to determine the porosity values of the etched surfaces. Gravimetric analysis is a destructive test that relies on the fact that freshly etched pSi dissolves rapidly in alkaline aqueous solutions (e.g., NaOH or KOH). The gravimetric measurements were performed by weighing the substrates before etch, after etch, and finally after the dissolution of the porous layer (2). Nonetheless, this technique is subject to some errors and limitations. Gravimetry assumes that the pSi film is macroscopically uniform, implying that the thickness of the film is the same across all the sample. This is not always the case, particularly for samples fabricated using long etching times where an observable gradient along the depth of the pores is evident from the center to the edges of the anodised sample. Another common error is the thinning of the film during extended etching procedures. This leads to the dissolution of the top of the surface consisting of fine filaments that dissolve in the electrolyte during the etching process, reducing at some extent the thickness of the layer and yielding to calculated porosities larger than the actual value. Finally, the instrument utilised to measure the mass of the substrates is prone to the introduction of inaccuracies in the readings collected throughout the weighing process of the samples (e.g., insufficient resolution).

Herein, further optimisation of the sample's fabrication was carried out to increase the loading capacities of structurally robust nanoporous tips. A reduced value in the electrical resistivity range of the crystalline silicon wafers (i.e., 0.8 to 1.0 mΩcm vs 0.55 to 1.0 Ωcm) was used to produce the porous surfaces. A non-destructive optical method was employed to calculate with high accuracy the porosity of the surfaces yielding to the construction of calibration curves governing the etching rate and porosity values as a function of the current density applied during the fabrication, to ultimately determine with high precision the physical thickness and loading capacity of the resulting surfaces.

### 2. Materials and Methods

All pSi surfaces were fabricated using heavily doped p-type silicon wafers with a resistivity range of 0.8-1.0 mΩ cm, (100)-oriented, boron (B)-doped, and with a thickness between 500-530 μm. Silicon wafer substrates were etched using an electrolyte mixture of 48 wt. % hydrofluoric acid (HF) in absolute ethanol (99.9%) in a volumetric ratio of 3:1, respectively. Electrochemical anodisation was performed using a Teflon cell by applying an electrical current (driven by a source meter unit) between the flat aluminum foil underneath the Si wafer and a platinum electrode. Briefly, the Si wafer was pre-treated by anodic etching at a specific current density for a particular fabrication condition (listed in Table 5) for 30 s to avoid the formation of a parasitic layer, which restricts the diffusion of the probe into the pSi layer. The pSi sacrificial layer was then dissolved by exposure to NaOH solution (1 M) for 120 s, followed by rinsing with ultrapure water and dried under a flow of nitrogen gas. The pre-treated surfaces were then etched to produce pSi surfaces by applying the current density and etching time values listed in Table 5. Next, the freshly etched pSi substrates were thermally oxidized at 400° C. for 1 h in air using a tubular furnace to confer hydrophilic properties to the anodised surfaces. The optical setup used for the porosity calculation consisted of a tungsten lamp (HL-2000, Ocean Optics) and a CCD spectrophotometer (HR2000+ES, Ocean Optics). A bifurcated optical reflection probe was coupled to both the white light source and the spectrophotometer, while its distal probe end was focused through a collimating lens onto the surface of the porous silicon layer at normal incidence. The spotlight (1 mm in diameter) was then focused on the center of the sample for spectroscopy measurements. Note: the samples characterised for the calculation of the porosity were etched as described above and using the etching conditions listed in Table 5 with an etching time of 120 s for all the etching conditions. Scanning electron microscopy (SEM) was conducted on FEI Nova NanoSEM 430 using an accelerating voltage of 5.0 kV. As-fabricated pSi surfaces were cut into small sections before SEM analysis. For FFT analysis, reflectivity spectra were recorded in the wavelength range of 500 to 1000 nm, with a spectral acquisition time of 100 μs. Typically, 10 spectral scans (1 s integration time) were averaged using SpectraView software (Ocean Optics). FFT values of the recorded reflectivity spectra were processed using IGOR PRO from Wavemetrics Inc.

TABLE 5
Etching conditions used to fabricate the pSi surfaces.

| | Current | Etching |
|---|---|---|
| Surface | density | time |
| No. | (mAcm⁻²) | (min) |
| 1 | 50 | 180 |
| 2 | 57 | 180 |
| 3 | 64 | 180 |
| 4 | 79 | 120 |
| 5 | 100 | 120 |

### 3. Porosity Calculation Using Spectroscopy Liquid Infiltration Method (SLIM)

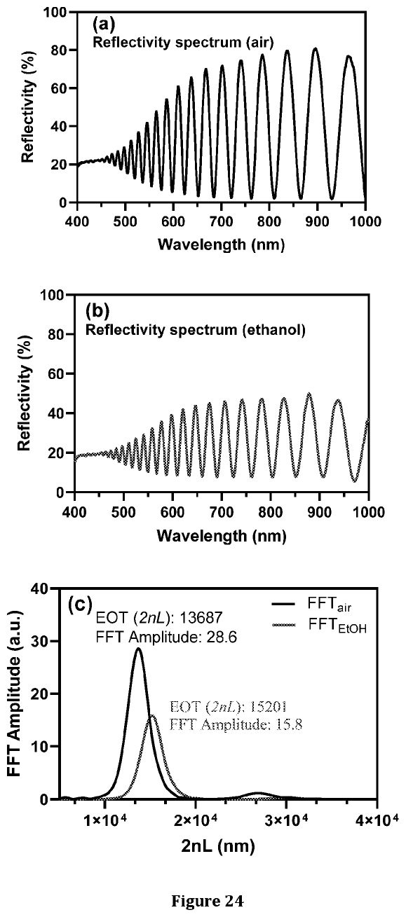

The porosity of the samples was determined using the spectroscopic liquid infiltration method (SLIM). SLIM is a non-destructive method based on spectral measurements of samples. By measuring the (reflectivity) spectrum of a porous film in air (e.g., air-filled pores) and with a liquid of known refractive index (n) filling the internal volume of the pores (i.e., ethanol, n=1.3611), the porosity, thickness, and n of the layer can be determined.² FIGS. 24 (a) and (b) show the reflectivity spectra of Surface 1 in air (n=1.0) and with the pores filled with ethanol (EtOH, n=1.3611), respectively. The fast Fourier transform (FFT) of each reflectivity spectrum yields to the product 2 nL (known as effective optical thickness, EOT) represented as a single-peak whose position and intensity along the x-axis correlate with the n of the pSi layer. Briefly, EOT=2 nL and the parameter thus contains information about the n and thickness (L) of the pSi film. L remains constant, and only n is susceptible to variation upon filling of the pores. When the liquid is admitted into the pores, the n of the porous film increases, producing a spectral shift in the fringe pattern (of the reflectivity spectrum) and, therefore, in the EOT value, as depicted in FIG. 24(c). The values of EOT and FFT (amplitude) are then fit to a two-component Bruggeman model of refractive index yielding values of porosity, thickness, and n of the porous layer. Appendix B provides the governing equations used for the SLIM. The SLIM analysis assumes that the medium inside the pores completely fills the pore volume. SLIM determines the physical properties of a pSi film based on the open porosity (i.e., the pore volume accessible to the solution), while the gravimetric method includes both the open and close porosities (e.g., the pore volume not accessible to the solution) (2). Therefore, SLIM provides a more realistic measure of the accessible volume within the nanostructure. In addition, the non-destructive nature of SLIM makes it ideal for quality control, for example, to verify the reproducibility of the pSi surfaces fabricated. The porosity and n values estimated using SLIM analysis for the pSi surfaces fabricated using the etching conditions listed in Table 5 are summarised in Table 6 (see below).

### 4. Morphological Characterization of the pSi Surfaces

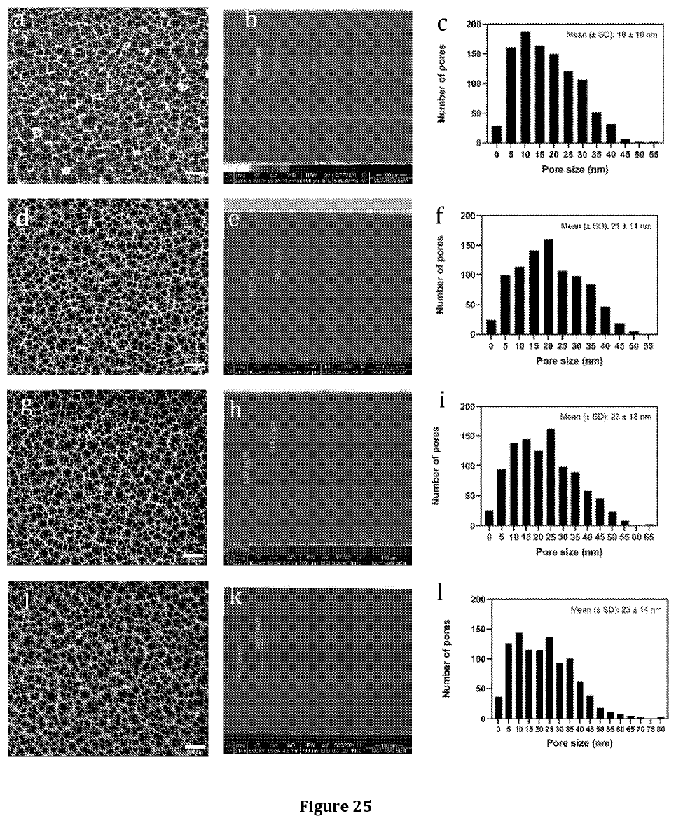

Morphological characterization of as-prepared pSi films (e.g., pore size and physical thickness) was performed via scanning electron microscopy (SEM) analysis. FIGS. 25 (a) and (b) show the top and cross-sectional views of a pSi film prepared using a current density value of 57 mA·cm⁻² for a total time of 180 min (Table 1, Surface 2) to a ˜530 μm thick silicon wafer. From these images, it can be observed a pore depth of ˜364 μm from the total thickness of the wafer and a calculated etching rate of ˜34 nm s⁻¹ (etching rate=pore depth in nm/etching time in s). Characteristic pore size distribution of the substrate is shown in FIG. 2 (c). Pore size histogram was obtained using the top-view SEM image for each surface, respectively. Thresholding of the grey-scale top-view SEM micrograph using ImageJ software yields binary images that allow for straightforward differentiation between the pores and the silicon pore wall framework. After noise removal using mathematical morphological openings and closings on the threshold image, pore areas in pixel count were extracted using the Particle Analysis method of ImageJ. The pore areas were then expressed in nm² by application of a conversion factor based on the ratio of the SEM scale bar pixel count to the corresponding length in nm. Finally, effective pore diameters were calculated from the extracted pore areas (3). FIG. 2 (c) depicts a pore size distribution between 5 to 55 nm with a mean (±SD) pore size of 18±10 nm. FIGS. 2 (d) to (l) show the top and cross-sectional SEM images and the pore size histograms of the pSi surfaces prepared using current densities values of 64 mA·cm⁻² for 180 min, 79 mA·cm⁻² for 120 min, and 100 mA·cm⁻² for 120 min, respectively. The observable pore depth for each substrate was ˜386, 314, and 330 μm, respectively, yielding etching rates of ˜36, 44, and 46 nm s⁻¹. Pore distribution was found to be between 5 to 55 nm, 5 to 65 nm, and 5 to 80 nm for each surface with an average pore size (±SD) of 18±10, 21±11, 23±13, and 23±14 nm, respectively. The pore histograms of FIG. 2 showed a clear trend. As the current density increased, the pore size became larger alongside an increment in the density of pores exhibiting larger dimensions than the average pore size (for each case). Therefore, increasing the porosity and the internal volume (e.g., loading capacity) of the surfaces.

### 5. Calculation of the Loading Capacity (LC) of the pSi Surfaces

The volume of the payload solution admitted within the pores is related to the porosity of the samples. Once the porosity is known for each substrate, a total spatial volume (V) was calculated (i.e., V=X·Y·Z). The spatial volume was obtained considering a dimension of tip of 5×5 mm² (i.e., X=Y=5 mm in length) in all cases, and Z is the depth of the etched pores in mm (i.e., Z=thickness in mm). The loading capacity (LC) in μL was determined by the product of the spatial volume (V) and the fractional porosity of each surface (i.e., LC=V*Porosity). The calculated values of the theoretical loading capacities for each surface (listed in Table 5) and their resulting physical properties are summarised in Table 6.

TABLE 6
Porous surfaces and their physical properties.

| | | | Etching | | | |
|---|---|---|---|---|---|---|
| | Pore | | rate | | | Loading |
| Surface | size | Thickness | (Vₑ) | Refractive | Porosity | capacity |
| No. | [nm]^{a} | [μm]^{a} | [nm s⁻¹]^{a} | index (n)^{b} | [%]^{b} | (LC)[μL]^{c} |
| 1 | 2-40 | 335 | 31 | 2.005 | 51.9 | 4.3 |
| 2 | 5-55 | 364 | 34 | 1.912 | 52.2 | 4.8 |
| 3 | 5-55 | 386 | 36 | 1.911 | 52.3 | 5.0 |
| 4 | 5-65 | 314 | 44 | 1.901 | 54.2 | 4.3/5.2* |
| 5 | 5-80 | 330 | 46 | 1.827 | 57.1 | 4.7 |

^{a}As determined via scanning electron microscope (SEM) analysis.
^{b}As determined by spectroscopic liquid infiltration (SLIM) method.
^{c}Total volume, V = X · Y · Z, where X = Y = 5 mm and Z = thickness of the film (in mm). Loading capacity is LC = V*Porosity.
*Calculated loading capacity for Surface 4 if the sample were fabricated exhibiting a thickness ~386 nm.

### 6. Calibration Curves for Porosity (P) and Etching Rate (Vₑ)

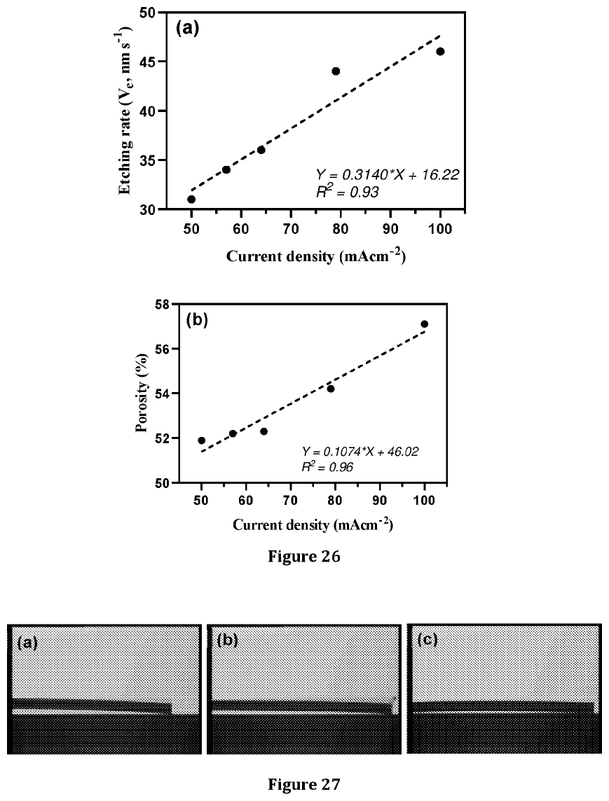

Table 6 summarises the most relevant physical characteristics of the as-fabricated pSi surfaces. By using these values, it is possible to construct calibration curves to calculate the porosity (P) and the etching rate (Vₑ) for specific values of the current density applied during the anodisation process (in the range between 50 and 100 mA·cm⁻²). The morphological features of the porous surfaces are highly dependent on the silicon wafer's parameters, such as the crystal orientation, the dopant element (impurities), and the electrical resistivity. Herein, we present the equations governing the estimation of the porosity (P) and etching rate (Vₑ) as a function of the obtained physical properties of Surfaces 1 to 5 (listed in Table 5). Fabrication of the surfaces is limited (in this case) to the use of heavily doped p-type silicon wafers with a resistivity range between 0.8 to 1 mΩcm. All other parameters remained constant throughout the fabrication process, such as electrolyte composition, orientation, and dopant element of the silicon wafer. FIG. 26 shows the calibration curves obtained for the etching rate and porosity. The current density of 100 mA·cm⁻² is limited (experimentally) to an etching time of 120 min to remain structurally stable. For etching times beyond 120 min, evidence of cracks or collapse was observed. Current densities below 100 mA·cm⁻² can resist thermal oxidation, dicing procedures, and ultrasound application without compromising its structural integrity.

### 7. Thermal Oxidation of the pSi Surfaces

The silicon hydride-terminated surface of freshly prepared pSi films is highly reactive to atmospheric conditions (e.g., oxygen and water), leading to oxidation and hydrolytic corrosion of the porous skeleton. Oxidation causes a significant change in the n of the material (n=3.5 for Si and n=1.4 for SiO₂) and consequently in the EOT values. Similarly, corrosion can lead to changes in porosity and film thickness, reducing EOT and leading to structural collapse. A stabilised/passivated surface is therefore required. Thermal oxidation is a convectional method to stabilise pSi surfaces. The controlled growth of the oxide layer can be used to effectively passivate the nanostructure by creating Si—OH or Si—O—Si terminated surfaces and at the same time rendering them hydrophilic to ease the admission of solution into the pores. The thickness of the oxide layer is adjustable and dependent on the temperature and humidity conditions. Herein, pSi surfaces were subjected to a mild thermal oxidation process of 400° C. for 1 h in air. At temperatures between 250° C. and 440° C. back-bond oxidation is the dominating reaction. Si—Si bonds oxidise by incorporating one oxygen atom into their back-bond, thus leading to SiO_{y}—Si—Hₓ surface species. With regard to thermal oxidation, the structural stress associated with the volumetric expansion of the crystal lattice can be observed to some extent in the porous substrates after the post-annealing treatment, as depicted in the photographs of FIG. 27. Although the surfaces are structurally robust to endure the increase of the skeleton volume by oxidation, it is desirable to mitigate the impact of the thermal stress as it can lead to macroscopic cracking or collapse of the structure. Ozone treatment represents an alternative for reducing the mechanical stress of the structure. However, the hydrophilic properties conferred to the surfaces will be retained temporarily before the surface returns to its initial surface energy conditions. In addition, cutting/dicing the parent pSi sample into small pieces (e.g., tips with dimensions of 5×5 mm²) before the thermal or ozone treatment can also relieve some of the induced structural stress. Finally, annealing of pSi surfaces using a controlled ramping and stabilization time of the temperature can also play an essential role in avoiding macroscopic deformation of the pSi samples.

### 8. Conclusions

Electrochemically anodised pSi layers can display a good macroscale uniformity when still wet but becomes fragile and, in some cases, even disintegrates as it dries in air. Particularly, when the porosity and/or the thickness is increased. Air-drying of the films leads to shrinkage, cracking, and pealing of the porous layer. The latter is caused by the build-up of capillary forces that arise from the pore liquid evaporation. Whether or not a given pSi surface will survive the effects of those stresses relies entirely on its mechanical properties. Our results showed that for films of fixed porosity and pore size densities, the onset of structural cracking and macroscopic collapse was strongly associated with the thickness of the layer. There is an optimum value for both the current density and etching time for which cracking/collapse of the air-dried film is absent. For example, current densities between 64 and 79 mA·cm⁻² and etching times of 180 and 120 min resulted in robust surfaces with payload capacities of up to 5.0 μL without compromising the sample's integrity. In addition, the resistivity range of the wafer also influences the determination of these critical values. Therefore, the fabrication conditions yielding stable pSi surfaces with increase loading capacities can be determined as a function of the silicon wafer specifications (e.g., semiconductor type, resistivity range, and dopant element) and etching conditions (e.g., the electrolyte composition). For instance, by using n-type silicon wafers or low doped p-type silicon wafers combined with specific etching conditions, pore sizes ranging from 30 to 90 nm can be obtained, thus increasing the porosity and loading capacity of the pSi surfaces. However, the sample's thickness must be meticulously determined in order to produce nanostructures capable of resisting structural stress. The latter accounts for the judicious selection of the wafer's resistivity when optimisation is required.

### REFERENCES

1. Canham, L. (Ed.) (2014). Handbook of porous silicon. Berlin, Germany: Springer International Publishing.
2. Sailor, M. J. Porous Silicon in Practice: Preparation, Characterization and Applications; WILEY-VCH Verlag GmbH & Co. KGaA, 2012.
3. Gaur, G.; Koktysh, D. S.; Weiss, S. M. Immobilization of Quantum Dots in Nanostructured Porous Silicon Films: Characterizations and Signal Amplification for Dual-Mode Optical Biosensing. Adv. Funct. Mater. 2013, 23 (29), 3604-3614

### 9. Appendix A. Pore Availability for Admission of Avastin

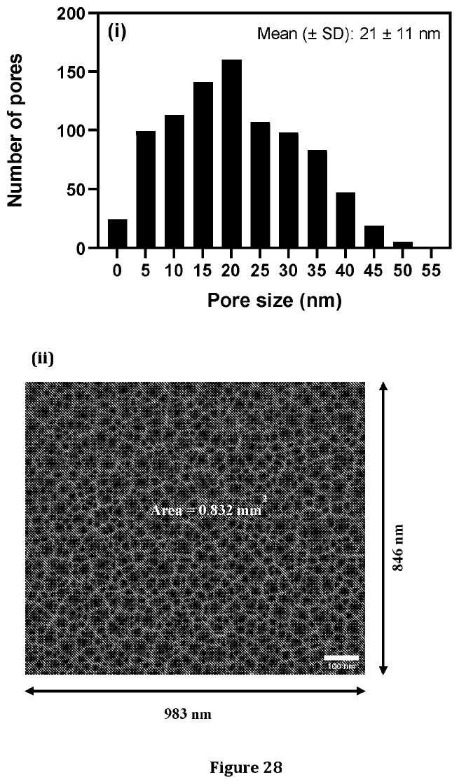

Avastin (MW 150 kDa) displays a hydrodynamic diameter of approximately 9.16 nm (1). Therefore, admission of Avastin into the porous scaffold is expected for pores featuring a diameter above 15 nm. The method followed to estimate the number of pores fulfilling this requirement is presented in FIG. 28. The pore histogram presented in FIG. 28 (i) shows the total number of pores (e.g., 897 pores) in an area of 0.832 mm² enclosed in the observable topography of the top-view SEM image (FIG. 28 (ii)) of the porous surface (Table 7, Surface 3). A relative percentage of 74% (e.g., 661 pores) from the totality of these feature the dimension required for Avastin diffusion into the nanostructure (Table 7).

TABLE 7
Pore distribution organised as a function of different diameters.

| Pore diameter | |
|---|---|
| (nm) | Frequency |
| 0 | 24 |
| 5 | 99 |
| 10 | 113 |
| 15 | 141 |
| 20 | 160 |
| 25 | 107 |
| 30 | 98 |
| 35 | 83 |
| 40 | 47 |
| 45 | 19 |
| 50 | 5 |
| 55 | 1 |

### REFERENCE

(1) Hirvonen, L. M., Fruhwirth, G. O., Srikantha, N., Barber, M. J., Neffendorf, J. E., Suhling, K., & Jackson, T. L. (2016). Hydrodynamic radii of ranibizumab, aflibercept and bevacizumab measured by time-resolved phosphorescence anisotropy. Pharmaceutical Research, 33(8), 2025-2032.

### 10. Appendix B. Spectroscopy Liquid Infiltration Method (Slim)

### Bruggeman Effective Medium Model

The Bruggeman model can predict the porosity and thickness of porous silicon and oxidised porous silicon. For a medium containing two distinct components the relationship is given by:¹

$$P \frac{{n}_{fill}^{2} - {n}_{layer}^{2}}{{n}_{fill}^{2} + 2 {n}_{layer}^{2}} + ( 1 - P ) \frac{{n}_{skeleton}^{2} - {n}_{layer}^{2}}{{n}_{skeleton}^{2} + 2 {n}_{layer}^{2}} = 0$$

Where P is the fractional porosity, n_{fill} is the refractive index of the medium filling the pores (air or liquid to be wavelength independent), nₛₖₑₗₑₜₒₙ is the wavelength dependent refractive index the skeleton that makes up the porous matrix (i.e., Si or SiO₂), and n_{layer} is the wavelength dependent refractive index of the composite porous silicon layer, incorporating both components. This equation can be solved for the refractive index of the composite layer:

$${n}_{layer} = \frac{1}{2} \sqrt{\begin{matrix}2 {n}_{skeleton}^{2} - {n}_{fill}^{2} - 3 {Pn}_{skeleton}^{2} + 3 {Pn}_{fill}^{2} \\ + \sqrt{8 {n}_{skeleton}^{2} {n}_{fill}^{2} + {( {n}_{fill}^{2} - 2 {n}_{skeleton}^{2} + 3 {Pn}_{skeleton}^{2} - 3 {Pn}_{fill}^{2} )}^{2}}\end{matrix}}$$

### Determination of Thickness and Porosity by SLIM

We can define the experimental observables EOTₐᵢᵣ and EOT_{liquid} as:

EOTₐᵢᵣ=n_{layer}L (measured in air)

And

EOT_{liquid}=n_{layer}L (measured in liquid)

Solution of Equation 1 for porosity in terms of Equation 3 and 4 yields:

$$P = 1 - \frac{[ {( \frac{{EOT}_{air}}{L} )}^{2} - {n}_{air}^{2} ] \cdot [ 2 {( \frac{{EOT}_{air}}{L} )}^{2} + {n}_{skeleton}^{2} ]}{[ 3 {( \frac{{EOT}_{air}}{L} )}^{2} ] \cdot [ {n}_{skeleton}^{2} - {n}_{air}^{2} ]}$$

$$And$$

$$P = 1 - \frac{[ {( \frac{{EOT}_{liquid}}{L} )}^{2} - {n}_{liquid}^{2} ] \cdot [ 2 {( \frac{{EOT}_{liquid}}{L} )}^{2} + {n}_{skeleton}^{2} ]}{[ 3 {( \frac{{EOT}_{liquid}}{L} )}^{2} ] \cdot [ {n}_{skeleton}^{2} - {n}_{liquid}^{2} ]}$$

### Example Three: Temperature, Frequency and Displacement of an Agent Carrier Aim

To measure the displacement, power and temperature of the agent carrier when connected to various bulk wave producing transducer types at fixed voltages for 10 minutes of continuous operation.

### Materials and Methods

For all measurements, the diameter of the circular tip of the agent carrier (as used in each of the Examples) that was connected to the transducer was 9.5 mm and weighed approximately 1.59 gram.

### 1. Displacement Measurements—Polytec MSA-400 Micro Systems Analyser

The characterisation completed in this study had been carried out with the aid of a Polytec MSA-400 Micro Systems Analyser located within the class 10,000 cleanroom. The MSA-400 Laser Doppler Vibrometer (LDV) is a precise optical transducer for determining the vibration velocity and displacement at a sample point.

The LDV uses the frequency shift of scattered laser light from a moving surface to calculate the velocity at the sample point. In this study scanning LDV was utilised, this is where the measurement point is moved across a defined grid on the surface of the device. Scanning LDV gives a more complete picture of the out of plane vibrational behaviour of the MμPharma device. The MSA-400 is able to capture displacement data at picometer levels of accuracy up to a frequency response of 24 MHz.

2. Temperature measurements—FLIR Thermal Imaging Camera

Temperature measurements taken throughout this study were conducted using a FLIR i7 thermal imaging camera. The camera has an accuracy of ±2° C. or 2%, can measure temperatures up to 250° C. and temperature differences or 0.10° C. The camera has a range of emissivity settings to choose from depending on the surface finish of the object being studied. In this study the semi-glossy setting was selected, this equates to an emissivity value of 0.8.

### 3. Ultrasonic Driver and Signal Generator

The transducers were powered using an ultrasonic driver/signal generator device that among other things, has high-speed resonance tracking of a series or parallel resonance modes, vibration amplitude control, and analysis functions such as impedance and frequency response measurement. This device displays a load power when driving the transducer this power value was used to estimate the W/Cm² of the agent carrier.

### Results

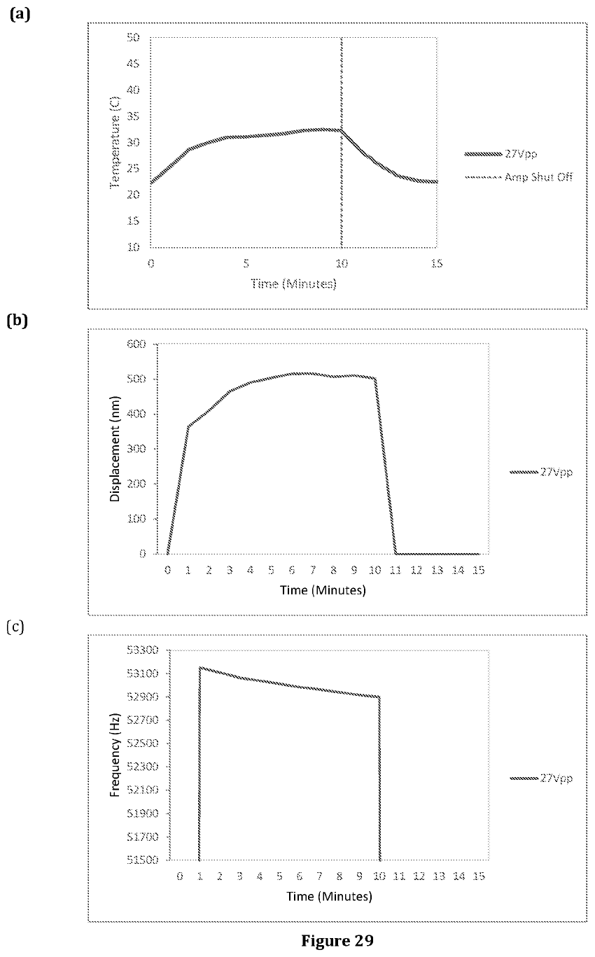

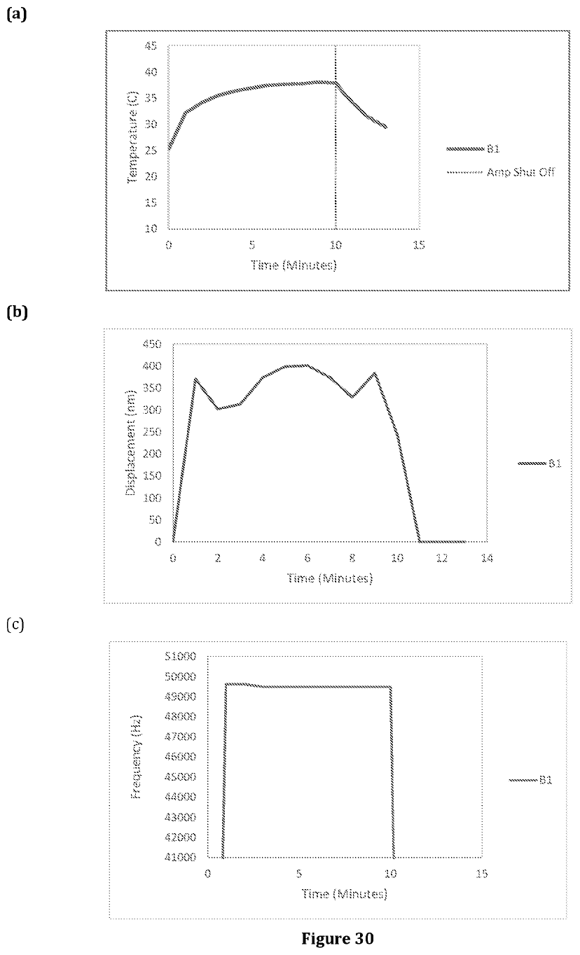

4. The Results of the Experiment are Shown in Table 8, FIG. 29 a-c, and Table 9, and FIG. 30 a-c.

For both trials the data shows a similar trend. There is a sharp increase in temperature over the first minute, just after the device is first turned on. After this initial increase, the temperature gradually plateaus until the signal generator is shut off. Throughout the experiment the frequency can be seen to drop, this is expected as the resonant frequency of a piezoelectric stack is known to reduce as the temperature rises [1].

In specific, FIG. 29 a-c shows data from driving one type of transducer in the device at 27 Vpp. The tip used in this study were milled. Table 8 and FIG. 29a show temperature of the agent carrier over operation time where 1) at 5 minutes of operation, a peak temperature of 31. 1° C. being a temperature rise of 8.8° C. and 2) peak temperature of 32.5° C. at 9 minutes of operation being a temperature rise of 10.2° C. Once the signal generator is shut off the temperature can be seen to decay exponentially. Table 8 and FIG. 29b show the output displacement of the device over operation time where the displacement can be seen to slowly increase until a plateau is seen around 500 nm. Table 8 and FIG. 29c shows the drive frequency calculated (to keep the voltage and the current in phase) by the signal generator over time where a steady drop in frequency from 53151.3 Hz to 52899.4 Hz occurred. A steady drop in resonant frequency is to be expected as the device heats up.

TABLE 8
Transducer voltage = 27 Vpp

| Time | | Displacement | Frequency | |
|---|---|---|---|---|
| (Minutes) | Temp (C.) | (nm) | (Hz) | Jpeg |
| 0 | 22.3 | 0 | 0 | 2407 |
| 1 | 25.4 | 363.9 | 53151.3 | 2408 |
| 2 | 28.7 | 410.1 | 53109.6 | 2409 |
| 3 | 30 | 465.2 | 53064 | 2410 |
| 4 | 31 | 490.3 | 53038.5 | 2411 |
| 5 | 31.1 | 503.5 | 53012.6 | 2412 |
| 6 | 31.4 | 515.2 | 52984.5 | 2413 |
| 7 | 31.7 | 516 | 52963.5 | 2414 |
| 8 | 32.3 | 506.6 | 52939.7 | 2415 |
| 9 | 32.5 | 510.5 | 52915.9 | 2416 |
| 10 | 32.3 | 501.7 | 52899.4 | 2417 |
| 11 | 28.4 | 0 | 0 | 2418 |
| 12 | 25.7 | 0 | 0 | 2419 |
| 13 | 23.6 | 0 | 0 | 2420 |
| 14 | 22.7 | 0 | 0 | 2421 |
| 15 | 22.5 | 0 | 0 | 2422 |

Table 9 and FIG. 30 a-c show data from driving one type of transducer in the device at 30 Vpp. The tips used during this trial were fabricated using Electron Beam Melting (EBM). Table 9 and FIG. 30a show temperature of the agent carrier over operation time where 1) at 5 minutes of operation, a peak temperature of 37° C. being a temperature rise of 11.7° C. and 2) peak temperature of 38.1° C. at 9 minutes of operation being a temperature rise of 12.8° C. After the signal generator is shut off the temperature can be seen to decay exponentially. Table 9 and FIG. 30b shows the output displacement of the device over operation time where the average displacement is 349.38 nm. When compared to the displacement data shown in FIG. 29b, the displacement can be seen to undulate. This undulation may be due to the surface finish of the tips being rougher that that used in the 27 Vpp trial detailed above. This can influence the efficiency of the coupling between the tip and the transducer and potentially lead to a less stable, noisier, displacement value as can be seen in FIG. 30b [1]. Table 9 and FIG. 30c show the drive frequency calculated (to keep the voltage and the current in phase) by the signal generator over time where a drop in frequency from 49630 Hz to 49500 Hz occurred.

TABLE 9
Transducer voltage = 30 Vpp

| | Time | | Displacement | Frequency |
|---|---|---|---|---|
| Jpeg | (Minutes) | Temp (C.) | (nm) | (Hz) |
| 2232 | 0 | 25.3 | 0 | 0 |
| 2233 | 1 | 32.2 | 371.2 | 49630 |
| 2234 | 2 | 34.2 | 302.5 | 49630 |
| 2235 | 3 | 35.6 | 313.6 | 49500 |
| 2236 | 4 | 36.4 | 374.2 | 49500 |
| 2237 | 5 | 37 | 399.2 | 49500 |
| 2238 | 6 | 37.5 | 401.4 | 49500 |
| 2239 | 7 | 37.7 | 374.1 | 49500 |
| 2240 | 8 | 37.8 | 329.2 | 49500 |
| 2241 | 9 | 38.1 | 384.8 | 49500 |
| 2242 | 10 | 37.9 | 243.6 | 49500 |
| 2243 | 11 | 34.1 | 0 | 0 |
| 2244 | 12 | 31.3 | 0 | 0 |
| 2245 | 13 | 29.5 | 0 | 0 |

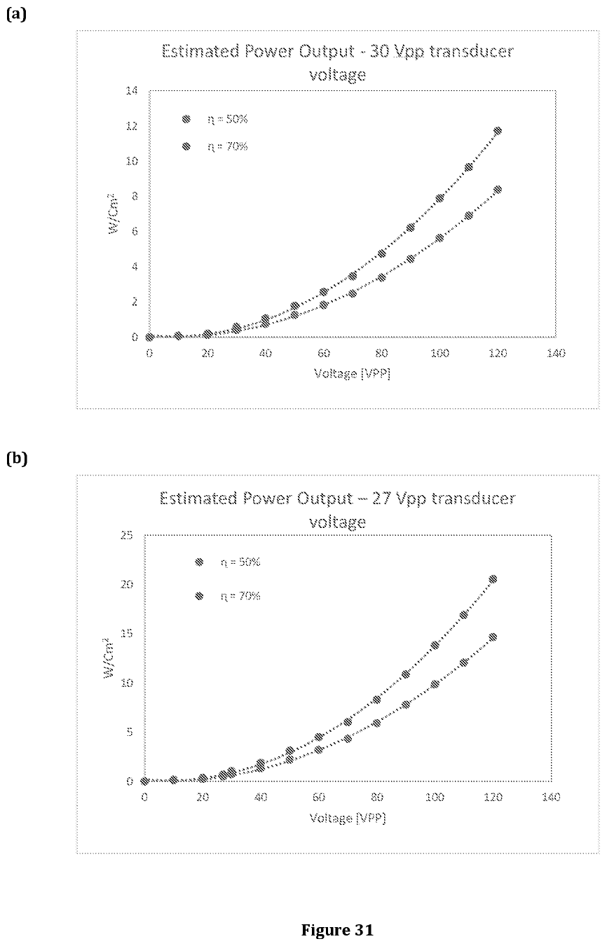

FIG. 31a-b shows the estimated irradiance of the tip in W/Cm². The output power was taken from the signal generator and divided by the surface area of the tip of the device. Ultrasonic energy is lost travelling from the piezoelectric stack to the device tip. This can be attributed to transducer losses, horn stress, interfaces and the thread attachment to the tip [2]. The graphs plotted are based on efficiency estimates of 50% and 70%.

### REFERENCES

[1] Mathieson, Andrew C. (2012) Nonlinear characterisation of power ultrasonic devices used in bone surgery. PhD thesis, University of Glasgow.
[2] Wang, K. et al. “Effect of interfacial preheating on welded joints during ultrasonic composite welding.” Journal of Materials Processing Technology 246 (2017): 116-122.

### Example Four: Analysis of Biological Activity of Insulin Released by Ultrasound

### Aim

The aim of this experiment was to determine whether 5 minutes of Mu Pharma device operation would affect the biological activity of insulin.

### Materials and Methods

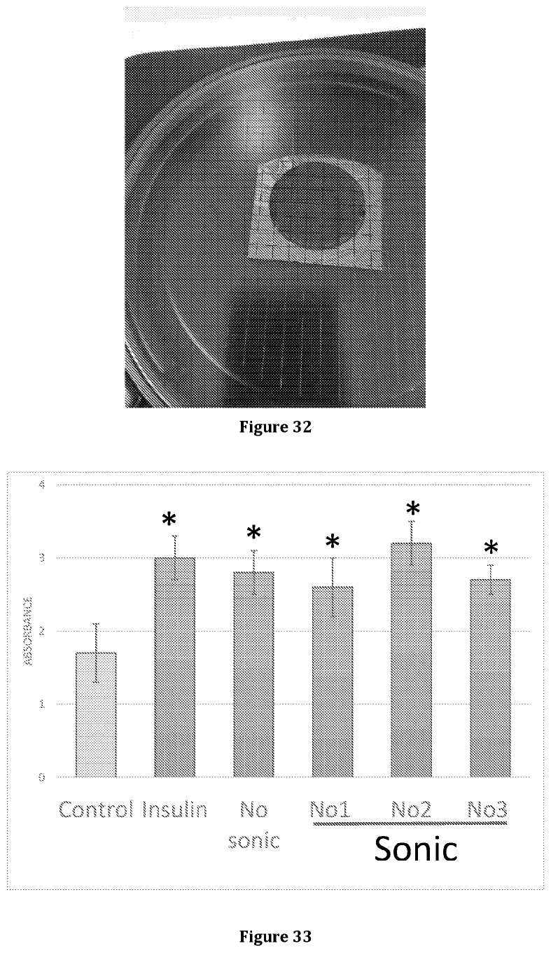

Standard insulin induced proliferation analysis in cells.
Device configuration—When operated, the voltage applied to the device transducer was 27 Vpp and the transducer operating frequency was approximately 50 kHz. (2) The diameter of the circular tip of the device that was connected to the transducer was 9.5 mm. No tip (including substrate) was used on more than one sample. (3) Within such tip, there was a 4.5 mm square shaped silicon substrate of the kind described in Example One above fastened that was loaded with ˜7 ul of Insulin. Multiple substrates (as used in this experiment) in a wafer configuration before dicing and loading was pictured in FIG. 32. (4) The tip of the device did not at any time during operation exceed 38 degrees centigrade as detailed in Example 3 above
   Control sample label: This is a known protein standard.
   Insulin standard: This is the human insulin stock (10 mg/ml), 500 μL.
   No sonic sample label: This is the control sample of a tip loaded with insulin at 10.0 mg/ml released into 90 μL of receiving saline solution.
   Numbered group (1, 2 and 3): These are the three test samples (all using the same method as described)

TABLE 10
Description of Samples

| Sample label | Description |
|---|---|
| No. 1, 2 and 3-three replicates | Insulin concentration of 10.0 mg/ml |
| | loaded into tip and after 5 minute device |
| | operation into 90 μL of receiving saline |
| | solution such solution was collected. |

### Results

Among other conclusions, absorbances of the sonic sample group (No1, No2 and No3) were not significantly different to the no-sonic control sample and the insulin standard, indicating that five minutes of device ultrasound operation does not affect the biological activity of insulin. (FIG. 33).

### Example Five: Live Rabbit Avastin Ocular Delivery of Avastin

### Aim

To histologically determine whether the MuPharma device is capable of non-invasively delivering Avastin into live ocular tissue.

### Materials and Methods

### Groups

(1) For the negative control, the device containing Avastin was applied 1-2 mm away from the corneal limbus without the generation of ultrasound, (2) For the test, the device containing Avastin was applied 1-2 mm away from the corneal limbus with the generation of ultrasound, and (3) For the positive control, direct injection of Avastin into the vitreous (current method of clinical application).

Device configuration

(1) When operated, the voltage applied to the device transducer is as provided in Table 1 below and the transducer operating frequency is approximately 50 kHz. (2) The diameter of the circular tip of the device that was connected to the transducer and was applied to the eye was 9.5 mm. No tip (including substrate) was used on more than one eye. (3) Within such tip, there a 4.5 mm square shaped silicon substrate of the kind described in section 7 of Experiment 1 (“Non-Invasive Drug Delivery Devices”) fastened that was loaded with Avastin. (4) The tip of the device does not at any time during operation exceed 38 degrees centigrade as detailed in Experiment 3 (“Temperature, frequency and displacement of an agent carrier”).

### Tip Application Site

A cautery device was used to mark all eyes of Rabbits 1 and 3 at a location approximately 3 mm away from the edge of the device tip application site. In Rabbit 2, no cautery was used but in order to locate the device tip application site, a suture was placed at the limbus 180 degrees opposite to the site of application.

### Fixation and Processing

Rabbits 1 and 3 eyes were immediately put into fixative after enucleation. The eyes for Rabbit 1 were subsequently processed into paraffin approximately nine months after fixation and those for Rabbit 3 were processed approximately one month after fixation. Rabbit 2 eyes were immediately put into fixative after enucleation and were processed approximately one week later. The eyes from all three animals (i.e., 6 in total) were fixed using formaldehyde. Representative sections from each eye were prepared and stained for routine morphology using the hematoxylin & eosin and PAS methods. Additional representative sections from each eye were subsequently immuno-stained using a fluorophore-conjugated antibody to human immunoglobulin (Ig). The fluorophore used is Alexa Fluor 647 (Molecular Probes). This dye displays a maximum absorption peak at approximately 647 nm and a maximum emission peak at approximately 670 nm (far red of visible spectrum). A fluorescent blue counterstain (DAPI) has also been applied. Additional sections from 4 out of the 6 eyes (Rabbits 1 and 2) were stained only with a blue nuclear counterstain (known as DAPI).

Since Avastin is a recombinant form of human immunoglobulin, any Avastin present within the rabbit tissue should theoretically be labelled with the fluorophore-conjugated antibody to human Ig.

### Imaging

All imaging of actual slides was performed with high-resolution, full-section scans being collected using either a Leica Aperio (for H&E and PAS) or an Olympus VS120 (immunofluorescence) slide scanner. The filter settings were set to enable optimal visualization of Alexa Fluor 647 and DAPI. Moreover, all scans were conducted using the same scanner settings (i.e., using the same gain and exposure times). A “yellow” pseudo-colour (technically a “look-up-table” or LUT) has been digitally applied to the Alexa Fluor 647 channel to provide clearer demonstration of the signal (since “red” and “blue” colour combinations are unsuitable for viewing by individuals with some forms of colour vision impairment). In lay language, this means that fluorescence associated with antibody binding to the tissue should appear “yellow”, rather than the natural “red” colour that might be seen with the naked eye down the microscope. Since it is common for tissue sections to display a degree of autofluorescence, the imaging of representative sections stained only with DAPI will provide a form of control. In other words, any “yellow” signal observed in the absence of the antibody having been applied, can be ignored.

Virtual microscopy of H&E-stained and PAS-stained sections was performed first, using Aperio ImageScope software. All slides were screened to identify those which contained the most complete portion of an intact corneal-limbal junction on both sides of the eye. Once identified, these sections were re-examined (at the highest magnification supported by the initial scan) for evidence of burn marks caused by the cautery device or a suture mark (since the device itself has not been found to induce notable changes in tissue structure). Representative images were captured using ImageScope. Labelled montages of images were created using Adobe Photoshop. Some images were rotated to provide a more consistent orientation. All images have been consistently adjusted to 300 dpi.

Virtual microscopy of slides scanned for fluorescence was performed using Olyvia software (Olympus). Sections obtained from the eye that was labelled as having received an intra-vitreal injection of Avastin (AVAS IV) was used as a positive control. The image histogram of intensity levels for the “antibody channel” (displayed within the image as “yellow”) was adjusted to a minimum value of “20” and a maximum value of “3000”. This same setting was subsequently applied to all other images within the study, including sections that only received the blue DAPI stain. The blue counterstain was also viewed at these same settings, with occasional slight adjustments to enable balance across different sections. Representative images were captured using the “Copy Display to Clipboard” tool and subsequently adjusted to 300 dpi using Photoshop. Some images have been cropped to enable better fit within a single montage. Labelling and montage creation were again completed using Adobe Photoshop.

TABLE 11
Delivery device parameters and tissue fixation/processing period
summary.

| | |
|---|---|
| Ultrasound | Period between tissue fixation |
| duration | and processing |

RABBIT 1

| | | |
|---|---|---|
| Right Eye Delivery | | |
| device | | |
| MuPharma Device | 1 minute 50 | Approximately 9 months between |
| (~7 ul tip pSi | seconds | tissue fixation and processing |
| capacity) 30 Vpp | | |
| applied to | | |
| transducer | | |
| Left Eye Delivery | | |
| device | | |
| MuPharma Device | 4 minutes 30 | Approximately 9 months between |
| (~7 ul tip pSi | seconds | tissue fixation and processing |
| capacity) 30 Vpp | | |
| applied to | | |
| transducer | | |

RABBIT 2

| | | |
|---|---|---|
| Right Eye Delivery | | |
| device | | |
| Intravitreal injection | N/A- | Approximately one week between |
| (~7 ul Avastin) | CONTROL | tissue fixation and processing |
| Left Eye Delivery | | |
| device | | |
| MuPharma Device | 5 minutes | Approximately one week between |
| (~7 ul tip pSi | | tissue fixation and processing |
| capacity) 27 Vpp | | |
| applied to | | |
| transducer | | |

RABBIT 3

| | | |
|---|---|---|
| Right Eye Delivery | | |
| device | | |
| MuPharma Device | 5 minutes | Approximately one month between |
| (~7 ul tip pSi | | tissue fixation and processing |
| capacity) 30 Vpp | | |
| applied to | | |
| transducer | | |
| Left Eye Delivery | | |
| device | | |
| MuPharma Device | N/A- | Approximately one month between |
| (~7 ul tip pSi | CONTROL | tissue fixation and processing |
| capacity) 30 Vpp | Device was | |
| applied to | placed on eye | |
| transducer | for 5 minutes | |
| | without | |
| | ultrasound | |

### Results

### Analysis of Overall Tissue Morphology

Initial screening of H&E-stained and PAS-stained slides revealed satisfactory preservation of the anterior segment for each eye. The posterior segment of eyes was generally less well preserved from fixation/processing with frequent signs of retinal detachment and fragmentation. Some occasional artefactual damage caused by tissue processing and microtomy (e.g., tissue fragmentation, section lifting and folding) was observed, especially in the case of the crystalline lens which is prone to this. Localized cautery burn marks were obvious for Rabbits 1 and 3, ranging from minor changes to the surface epithelium (Right eye for Rabbit 3), up to near perforation of the anterior chamber (AC) at the corneal-limbus in the Right eye for Rabbit 1.

The Right eye of Rabbit 2 was used as a positive control for subsequent analyses of immunofluorescence compared to the Left eye of Rabbit 2. Only images of H&E-stained sections were recorded since no additional information was gained from those stained using the PAS method.

Based upon the above assessment, a table was constructed summarizing: (1) the section level and section number (e.g., 4/1) identified from the H&E-stained section as being optimal for starting the investigation, (2) the fluorescence image number (as named within the collection of images) corresponding to the next slide in the series for each block for starting the investigation, (3) any sections for each animal that were stained using DAPI alone. This information is presented below in Table 2.

TABLE 12
Summary of principal images used to perform the analysis.

| | AVAS IV | | AVAS US | | | |
|---|---|---|---|---|---|---|
| | Rabbit 1 | | Rabbit 2 | | Rabbit 3 | |
| | Right | Left | Right | Left | Right | Left |
| 1. H&E | 4/1* | 1/1 | 5/1 | 1/1 | 4/1 | 5/1 |
| 2. Alexa | 4/2 | 1/2 | 5/2 | 1/2 | 4/2 | 5/2 |
| Fluor | Image 11† | Image 1 | Image 20 | Image 25 | Image 13 | Image 23 |
| 3. DAPI | Image 35 | Image 34 | Image 37 | Image 36 | Not | Not |
| alone | | | | | provided | provided |

*Numbers denote level within the block followed by the section number for the level (as labelled on the slide).
†Image number as provided within the relvant file name-.

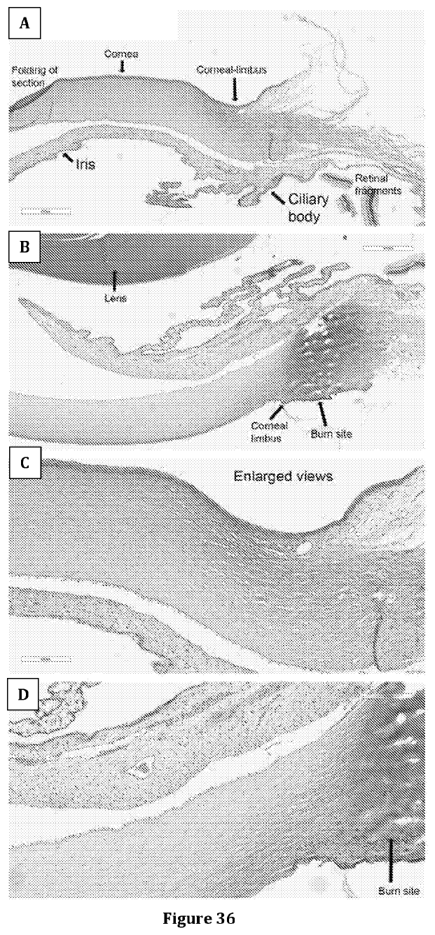

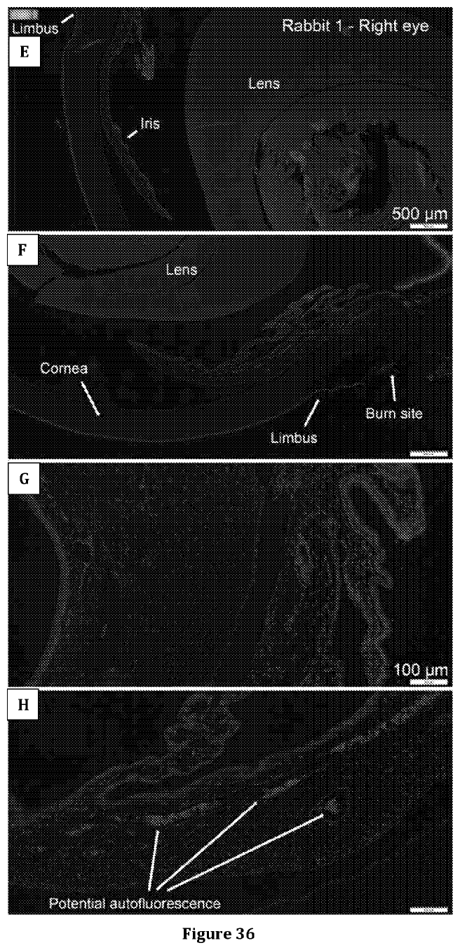

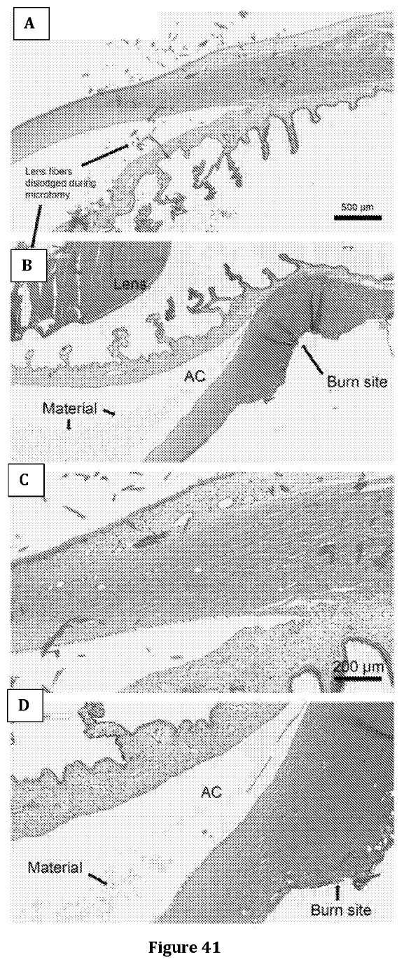

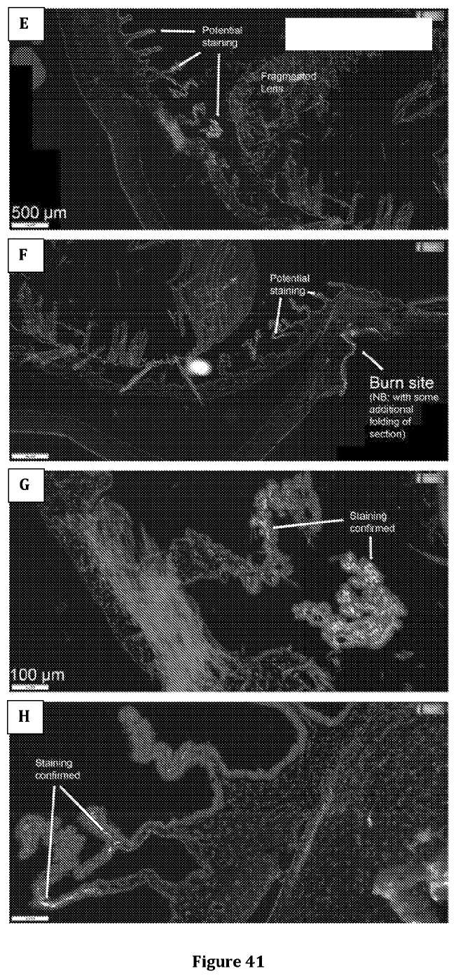

Composite images summarizing the gross morphological features for each eye (as demonstrated by H&E staining) are FIGS. 36-41.

A brief summary for each eye is as follows:

Rabbit 1—Right eye: The morphological features of the anterior segment are well-presented. A full-thickness cautery mark burn is present within the corneal-scleral junction on one side. Some fragments of retinal tissue are also present. This fragmentation is consistent with tissue fixation and processing artefacts. The neural retina was fully detached and frequently fragmented.

Rabbit 1—Left eye: The morphological features of the anterior segment are well-presented. A partial-thickness cautery mark burn is present (˜50% depth) within the corneal-scleral junction on one side. The neural retina was fully detached and fragmented

Rabbit 2—Right eye (AVAS IV): The morphological features of the anterior segment are well-presented. Some red blood cells and non-cellular eosinophilic material can be observed within the anterior chamber (AC). This material is suspected to be mixture of injected Avastin combined with fibrin and serum proteins. The neural retina was partially detached and fragmented.

Rabbit 2—Left eye (AVAS US): The morphological features of the anterior segment are well-presented. Some damage is evident through the corneal-scleral margin on one side. The appearance of this damage is consistent with that caused during insertion of a suture and thus was determined to be the opposite side from which the device was applied. The healthy appearance of tissue on the opposing treated side, suggests that no damage is caused by the US device. The neural retina was partially detached and fragmented.

Rabbit 3—Right eye: The morphological features for the anterior eye segment are again generally well-presented. The corneal epithelium has detached slightly in one place which coincides with distortion of the corneal stroma and so is likely a processing artefact (e.g., compression of tissue within processing cassette). Examination of several sections for this tissue confirmed slight changes in the morphology of the limbal epithelium on one side. These changes are consistent with those associated with a superficial cautery mark burn injury to the epithelial cells. Some eosinophilic material (protein) is present within the anterior chamber. The neural retina was the best preserved of all eyes examined with occasional shrinkage being observed (likely a processing artefact caused by shrinkage of tissue) resulting in either separation of the photoreceptor layer from the retinal pigment epithelium (RPE), or separation of the RPE from the adjacent choroid, or within the choroid itself.

Rabbit 3—Left eye: The morphological features for the anterior eye segment are again generally well-presented. The brittle nature of the fixed and processed lens tissue has resulted in fragments of broken lens fibers covering other parts of the section. A prominent cautery mark is observed on one side through the corneal-scleral junction, extending to a depth of approximately 60%. Some traces of eosinophilic material (protein) are again present within the anterior chamber. The neural retina was better attached than for that observed in Rabbits 1 and 2, with some occasional artificial separation of the photoreceptor layer from the RPE, or between the RPE and the adjacent choroid.

### Analysis of Slides Scanned for Fluorescence

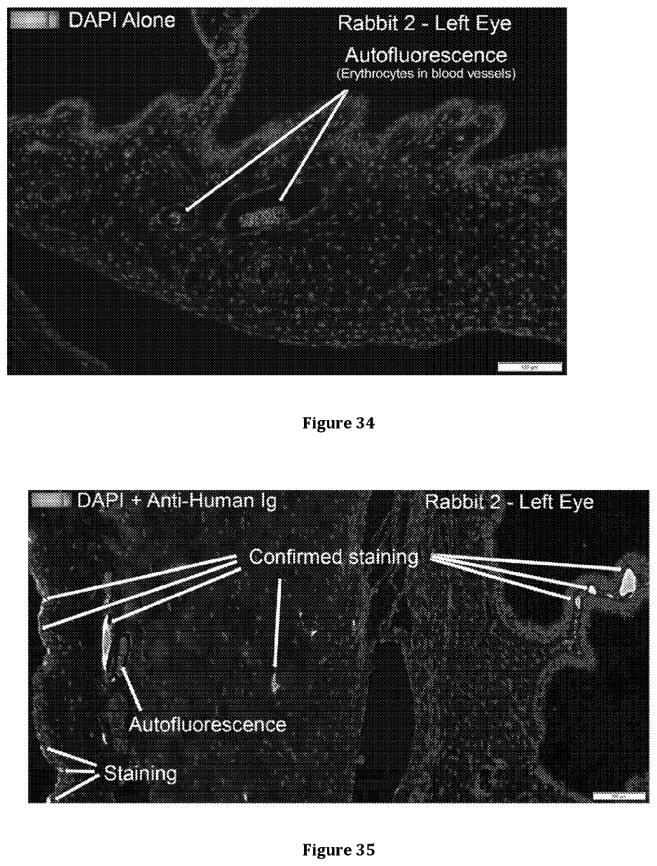

Two control sections were initially evaluated in order to validate the results observed for other sections. The section obtained from the Right eye of Rabbit 2 had reportedly (based upon labelling) received an intra-ocular injection of Avastin (i.e., Positive Control). The fluorescence settings for this image were therefore adjusted to obtain a standardized intensity profile for all other images. The same settings (Min20/Max3000) were subsequently applied to a section derived from the Left eye of Rabbit 2 which had only been stained using the blue fluorescent nuclear stain (i.e., DAPI negative control). The three other sections stained with DAPI-alone contained insufficient tissue to conduct a meaningful comparison. Examination of the negative control confirmed the presence of a low level of autofluorescence emanating from erythrocytes, as demonstrated below in the following high-power image (FIG. 34).

On the basis of this observation, any similar patterns of staining observed in sections from animals treated with the Avastin could be ignored as autofluorescence.

In direct contrast to the image displayed above, some test sections (such as that displayed below in FIG. 35) presented with areas of yellow colour which was determined to provide confirmation of immunoreactivity towards human-Ig (and thus Avastin) based upon the following criteria.

1. The intensity of yellow colour was noticeably higher than that observed for red blood cells (rbc) present in the sections stained with DAPI alone. The difference in intensity was determined using ImageJ image analysis software to be approximately 1.5 to 2-fold greater.

2. The morphology of yellow-coloured material when viewed at highest magnifications was different to that of red blood cells, appearing as either: (a) attached to the surface of tissue as a layer of homogenous material, (b) within the cytoplasm of cells, or (c) filling the lumen of blood vessels. Nevertheless, in the case of staining seen within blood vessels, the overall intensity of colour will be a combination of background (i.e., rbc) as well as Avastin.

Representative fluorescence micrographs for each eye are provided in FIGS. 36-41 (arranged in sequence by Rabbit No./eye). A summary of immunostaining observed within each eye is as follows:

### Rabbit 1—Right Eye:

The 9 month period between tissue fixation and processing of this eye is likely to have reduced immunoreactivity. This tissue appears to be generally negative for human Ig. Only background autofluorescence is observed throughout internal structures including the ciliary body and retina. Occasional brighter patches (possible drying artefact) are observed on the ocular surface.

### Rabbit 1—Left Eye:

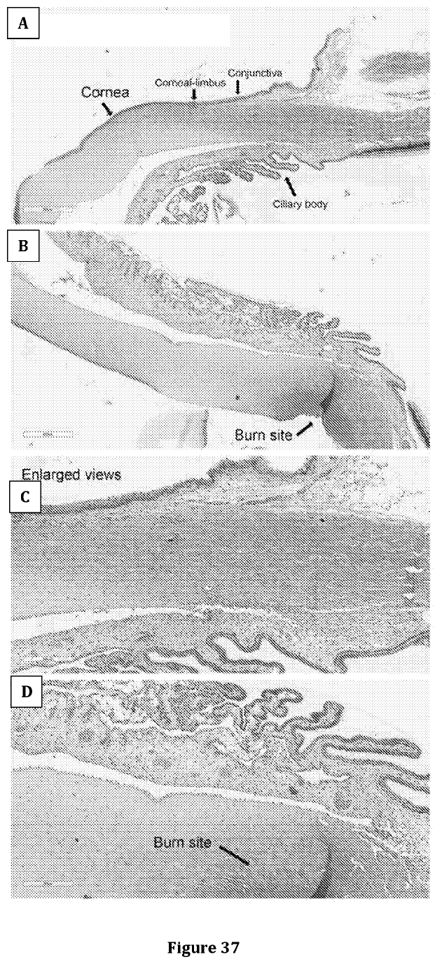

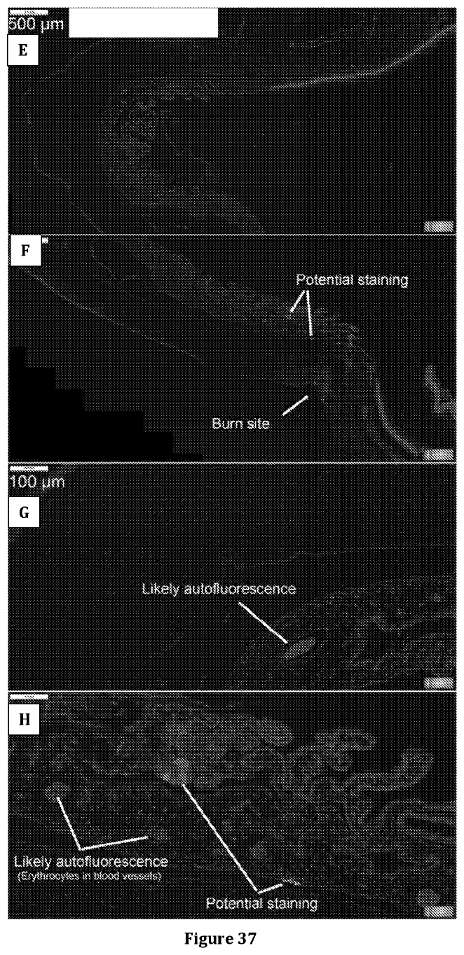

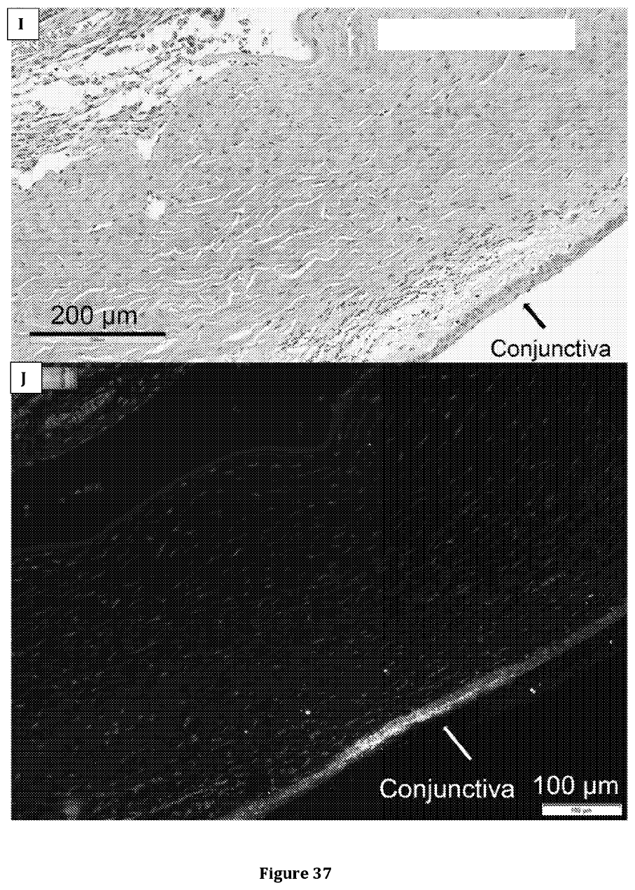

The 9 month period between tissue fixation and processing of this eye is likely to have reduced immunoreactivity. A small area of intense staining is observed on the anterior surface of the ciliary body in Image 1. A slightly larger and more diffuse area of staining is observed in Image 1 within some blood vessels of the ciliary body which appears to be above that associated with the autofluorescence of erythrocytes but might possibly be due to non-specific staining of the fluorescent antibody. FIG. 37 (second fluorescent panel for this eye) displays a bright patch of apparent staining within the conjunctiva. No staining is evident anywhere within the retina or choroid.

### Rabbit 2—Right Eye (AVAS IV):

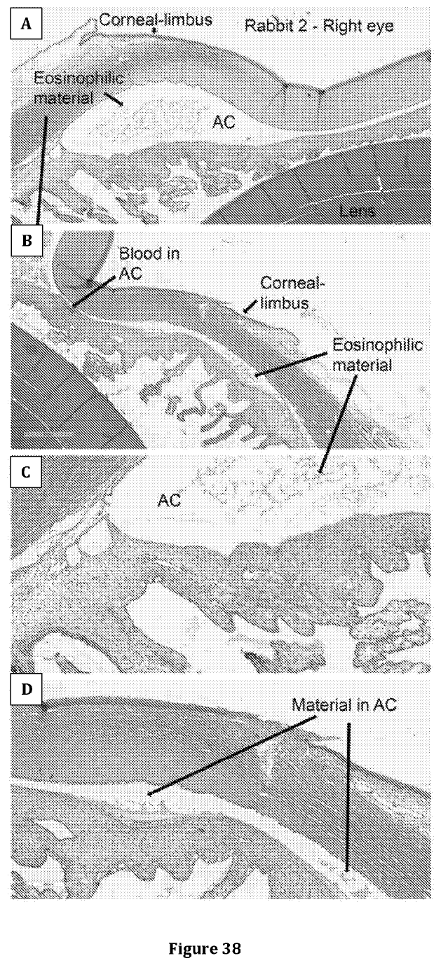

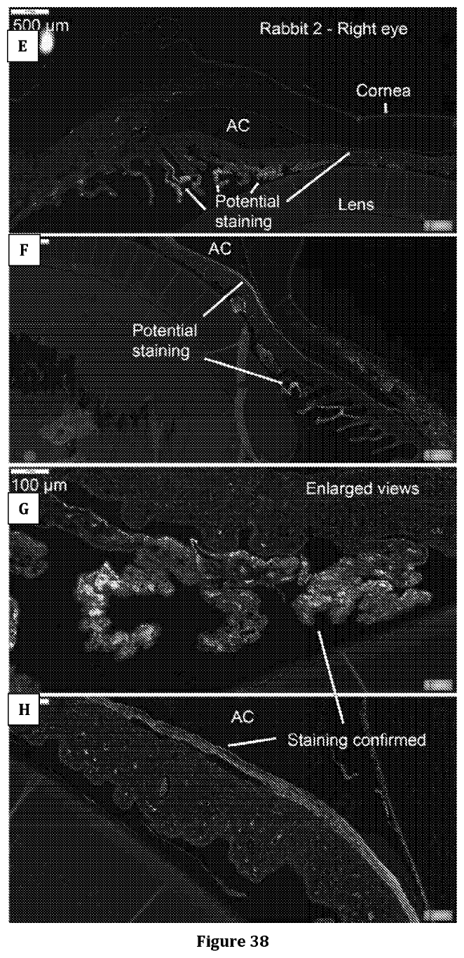

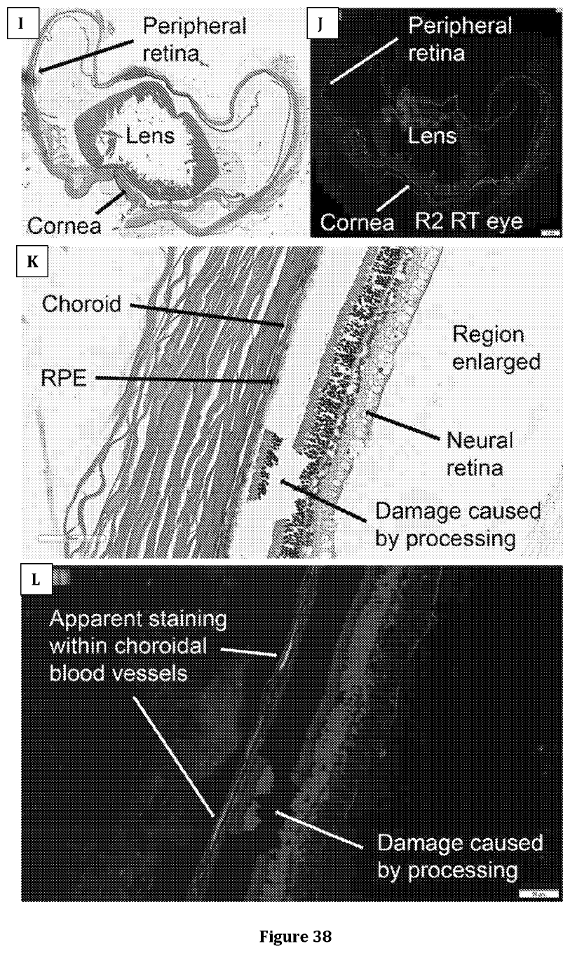

Marked immunostaining is observed throughout blood vessels of the ciliary processes. A thick film of immunoreactive material is present across the anterior surface of the ciliary body and extending onto the iris. Less evidence of staining was noted in areas approximately corresponding to the more diffuse eosinophilic material noted in the anterior chamber (AC), suggesting that this is mostly composed of serum proteins. Some evidence of immunostaining is also seen within choroidal blood vessels (as displayed in the enlarged images in FIG. 38).

### Rabbit 2—Left Eye (AVAS US):

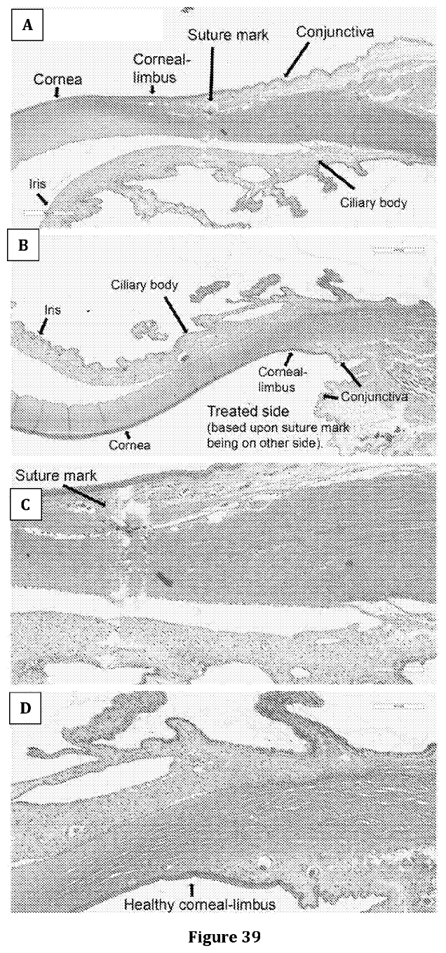

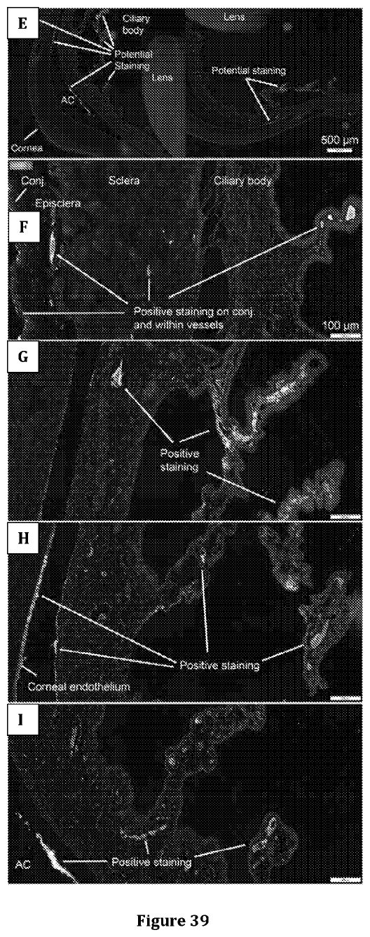

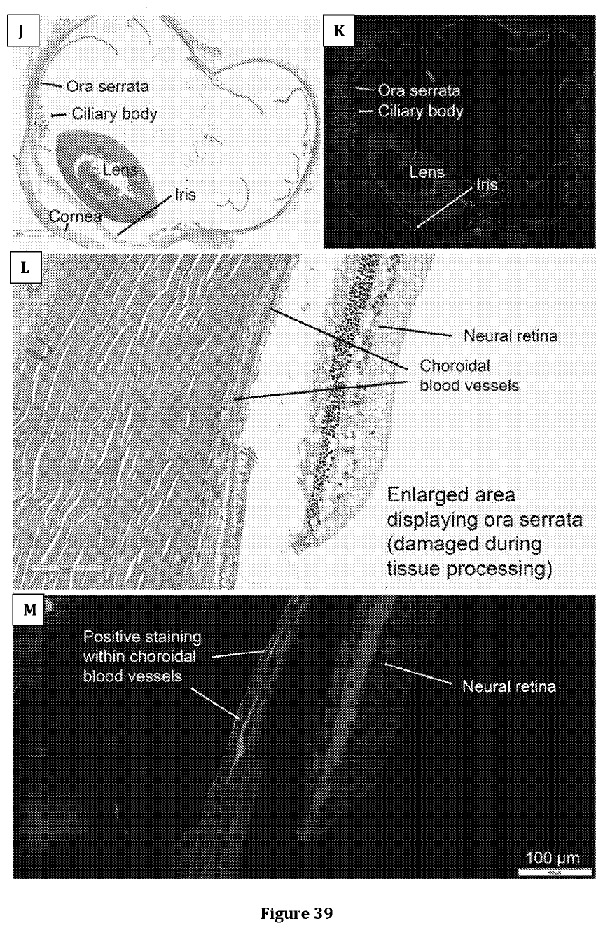

Intense levels of immunoreactivity are observed within multiple discrete areas. At low power view, immunoreactive material is visible within the anterior chamber and ciliary processes. Examination at higher power confirms the presence of positive staining on the conjunctival surface as well as within blood vessels of the episclera, sclera and ciliary body and ciliary processes. A patch of bright staining is also observed within the corneal endothelium and the opposing anterior surface of the iris. Evidence of immunostaining is also seen within choroidal blood vessels (as displayed in the enlarged image of ora serrata—FIG. 39).

### Rabbit 3—Right Eye (i.e., Eye 5):

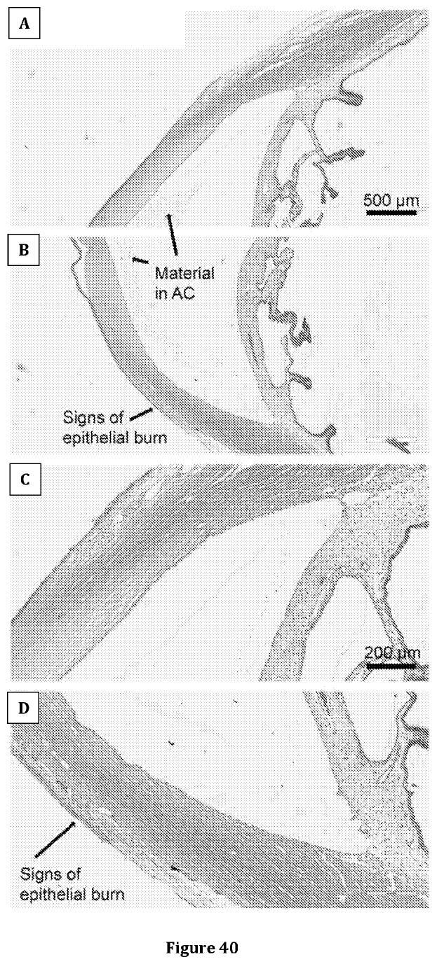

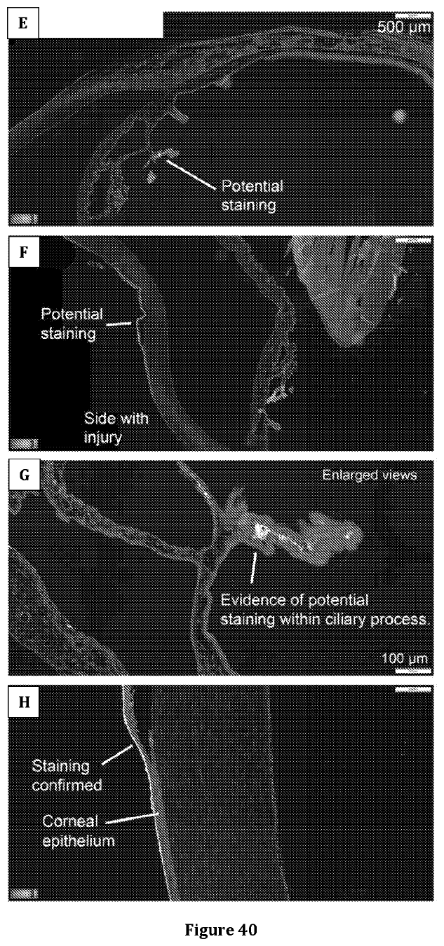

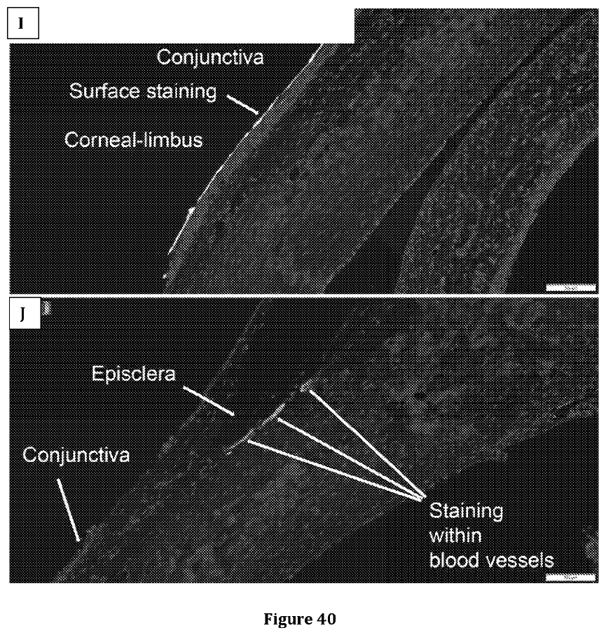

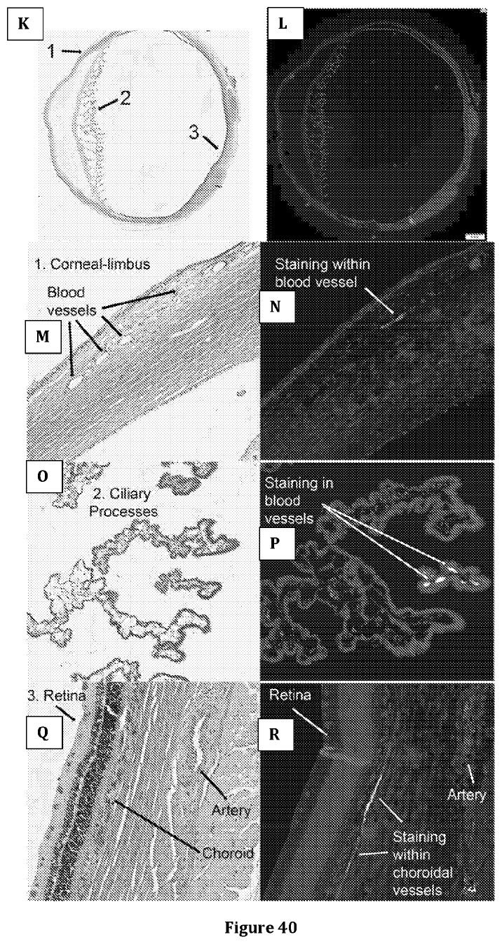

Intense staining for human Ig is present on a portion of the peripheral corneal epithelium, the adjacent conjunctiva (Images 12 to 15) and lesser evidence of staining within ciliary body (noted in at least one ciliary process as displayed in FIG. 40 (a-r)). Other sections from within this series display variable staining, but Image 12 displays evidence of staining across multiple levels including the conjunctiva, ciliary processes and within choroidal blood vessels at the back of the eye (FIG. 40).

### Rabbit 3—Left Eye (i.e., Eye 6):

Some sections display evidence of immunostaining within the ciliary processes (Image 23). Occasional staining is seen attached to the edges of tissue that separated within the choroid (e.g., Image 19). Some of this apparent staining may be due to drying of the fluorescent antibody onto the edges during application, but it is difficult to discount all staining seen as artefact given similar appearance to areas of confirmed staining. The prominent cautery mark in this eye should be taken into account as a potential alternative route by which some Avastin may have entered the eye (i.e., some Avastin may have remained on the surface of the eye following enucleation and then entered the eye via the wound prior to fixation).

### DISCUSSION & CONCLUSIONS

The above analysis provides convincing evidence of positive staining for human Ig within the eye of rabbits. Since Avastin is likely to be the only source of human Ig present, it can be assumed that the staining is due to the presence of this anti-VEGF drug. The above analysis also provides convincing evidence that the device is non-invasive. The intense bevacizumab-immunoreactivity observed within the eyes of animals treated with the device using ultrasound confirmed that bevacizumab was effectively delivered into the blood vessels within the eye. In addition, the positive staining with the fluorescent detection-antibody used to identify the humanised monoclonal antibody, bevacizumab, further confirmed that bevacizumab had retained its protein structure and was not affected by the device or operation of it.

Images derived from the Left eye of Rabbit 2 provides positive evidence for an non-invasive increase in Avastin uptake into rabbit eyes when the ultrasonic device has been applied. Strongest staining is observed within blood vessels, but there is also evidence of free Avastin being present within the anterior chamber (AC) and either adhering to, or being up taken into, adjacent tissues.

Rabbit 1 displays least evidence of Avastin uptake, with no staining being observed within the right eye of this animal and a borderline level of staining observed in the left eye.

The right eye of Rabbit 3 displays evidence of staining at multiple areas. The areas of staining with this Rabbit's left eye are less consistent. Some consideration should be given to potential penetration of the Avastin via the wound created by using the cautery device.

### Example Six: Investigation of Avastin Integrity

Example Six is a prophetic Example.

### 1.1. Coomassie Staining for Protein Integrity Method.

The Coomassie Brilliant Blue dyes are used for the quantification of protein, and work by binding to proteins through Van der Waals attractions and ionic interactions between sulfonic acid groups of the dye and positive protein amine groups.

Coomassie staining will be performed on saline buffer solutions, containing Avastin or Bovine Serum Albimum (BSA) as relevant that at least, is extracted from tips (agent carriers) following the steps provided below:

1. Control BSA tips will be loaded with either 2 mg/ml or 4 mg/mL BSA. Control will involve applying the device for 90 seconds (without ultrasound) or such longer duration as performed in any BSA test described below;

2. Test BSA tips will be loaded with either 2 mg/ml or 4 mg/mL BSA. Tests will involve operating the device (using a device setup as described in the live rabbit trial in Example 4) in durations of at least 15, 30, 45, 60 and 90 seconds;

3. Control Avastin tips will be loaded with 25 mg/ml Avastin. Control will involve applying the device for 90 seconds (without ultrasound) or such longer duration as performed in any Avastin test described below; and

4. Test Avastin tips will be loaded with 25 mg/ml Avastin. Tests will involve operating the device (using a device setup as described in the live rabbit trial in Example 4) in durations of at least 45, 60 and 90 seconds.

Briefly, after Polyacrylamide gel electrophoresis, the polyacrylamide gel will be incubated in the Coomassie Blue solution for 2-4 hours, until the gel will be uniform in colour and then the gel will be distained until background is clear. The gel will be then scanned (e.g. using a Canoscan 8800F) and molecular weight and relative intensities determined by comparison against a known protein standard.

### 1.2. Coomassie Staining for Protein Integrity Results

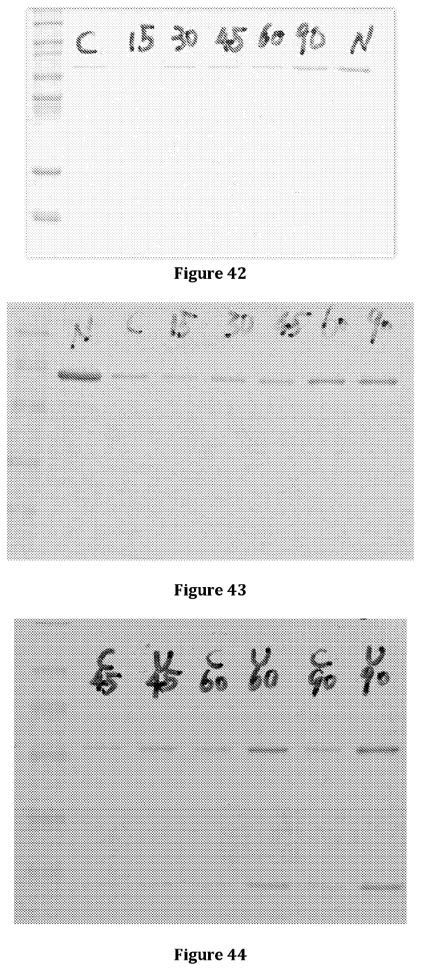

Coomassie blue staining of saline buffer solutions that underwent ultrasound application with 2 mg/ml or 4 mg/ml loaded BSA tips are expected to reveal no fragmentation of the protein due to ultrasound, indicated by absence bands at low molecular weights (expected result provided in FIG. 42 and FIG. 43 below).

Likewise, it is expected with saline buffer solutions that underwent ultrasound application from device with 12.5 mg/ml loaded Avastin tips, Coomassie blue staining of the polyacrylamide gels, indicate no abnormal fragmentation of the Avastin immunoglobulin aside from unavoidable SDS denaturation (expected result provided in FIG. 44 below).

### Example Seven: Determination of Whether Administration of LAIV Using the MuPharma Device Generates an Elevated Vaccine Immune Response

Example Seven is a prophetic Example.
Aim: To determine whether the administration of live attenuated influenza vaccine (LAIV) generates influenza specific immunity following delivery with the MuPharma device

### Timeline of Mouse Experiment

### Day 0

Group 1: (n=3) Naïve
Group 2: (n=5) LAIV—intranasal delivery (10⁴ PFU/10 μl)
Group 3: (n=10) LAIV—muPharma delivery (10⁴ PFU/10 μl)

### Day 20

Harvest nasal tissue, lung, spleen, and lymph node tissue for quantification of influenza vaccine specific T cells
Collect serum—store for measuring antibody response

### Outcome:

We will be able to show the size of the influenza specific T cell response elicited by LAIV following delivery with the muPharma device.

TABLE 12

| Deliverables | Positive Outcomes |
|---|---|
| Quantitate the number and type | Minimum: Compared to naive control, |
| of influenza reactive (NP and PA- | Mu Pharma device elevated tissue |
| specific) CD8⁺ memory T cells in | resident influenza specific T cells in the |
| the upper and lower respiratory | lung, T cells in the upper airways, and |
| tract and lymphoid tissue (spleen | T cells in the spleen. |
| and lymph nodes) | |
| Quantitate the number and type | Additional positive outcome |
| of influenza reactive (NP and PA- | In addition to the above minimum |
| specific) CD8⁺ memory T cells in | outcome, a positive outcome would |
| the upper and lower respiratory | also involve MuPharma device |
| tract and lymphoid tissue (spleen | generating ≥10⁴ tissue resident |
| and lymph nodes) | influenza specific T cells in the lung, |
| | ≥10³ T cells in the upper airways, and |
| | ≥10⁴ T cells in the spleen. |
| | Previous studies (not involving the |
| | Mu Pharma device) have shown that |
| | lodging flu specific T cell pools of this |
| | size, in these tissue compartments, is |
| | protective against influenza virus |
| | challenge. |
| Collect blood and separate serum | Store serum samples for future analysis |
| | if required |

### Example Eight: Live Mouse Chlamydia Vaccination Trial

Example Eight is a prophetic Example.

The purpose of this experiment is to determine whether the MuPharma device is capable of generating an immune response through performing a live mouse Chlamydia vaccination trial. This trial will at least involve a comparison of the MuPharma device loaded with a one protein antigen (plus an adjuvant) that is applied to the buccal mucosa versus an intraperitoneal injection of an adjuvant without such protein. Such trial will at least involve:
testing for reduced shedding of chlamydia in the subsequent vaginal challenge;
test for pathology outcome of the vaccination-challenge model compared to controls by the gross measure of hydrosalpinx;
test for mucosal IgG and IgG in vaginal lavage samples during and after vaccination and during the challenge;
collect and store blood and various tissues relevant to future analysis; and
option for immediate T cell responses from local reproductive tracts at experimental end point (pooled for all 10 animals for each separate group).

### Work Outline

It is expected that the work program described above will at least involve the following:
mouse vaccination running a total of 70 days.
The mice are allowed to settle for 1 week. On:
   day 7—test group will receive by the mupharma device applied to the buccal mucosa, a vaccination prime (possibly a major outer membrane protein (MOMP)) antigen plus adjuvant (possibly cpg)) and the control group will receive by a intraperitoneal injection an adjuvant only (same as used in test group) without antigen);
   day 21—each group will receive a repeat of their respective doses given on day 7;
   day 28—progesterone dose will be given to each group to synchronise menses; and
   day 35—vaginal infection challenge to each group with purified live Chlamydia Muridarum.
The key output is to observe a reduced vaginal shedding of chlamydia in total and at the infection peak. This will be monitored by swabbing and measuring the vaginal shedding of chlamydia every 3 days during peak and later stages 5 days for 35 days until day 70.
Pathological responses to chlamydial vaccines is also a risk that needs to be measured as an output during vaccine trials, so at completion of the experiment, gross reproductive pathology will be examined (measure of any hydrosalpinx). This can be visually measured with an option to dissect out the reproductive tissues for histological or molecular analysis.
A report showing the graph and data of vaginal shedding of chlamydia and gross pathology by organ measures (hydrosalpinx) between the two treatments. An interim dataset of day 6 shedding, the peak of infection, will be provided to facilitate further analyses (e.g. T cells).

### Timing of Work

The following Table shows the timing of the work:

TABLE 13
projected timing of the work

| | Month | Month | Month | Month |
|---|---|---|---|---|
| | 1 | 2 | 3 | 4 |
| Phase 1: Mice Vaccinations | | | | |
| Task 1.a Mouse model-5 weeks settle and vaccinate | | | | |
| Task 1.b Progress Report will consist of overall well | | | | |
| being of mice and tail bleed and vaginal lavage | | | | |
| antibody titres to the antigen. | | | | |
| Phase 2: Challenge Model | | | | |
| Task 2.a Day 6 shedding comparison dataset to be | | | | |
| provided | | | | |
| Task 2.b Surgery, final analysis and data | | | | |
| Task 3 Final report | | | | |
| 1. Clearance of vaginal chlamydia in the challenge | | | | |
| model, testing for a difference in mice vaccinated | | | | |
| with Mu Pharma device delivered antigens to those | | | | |
| vaccinated with buffer. | | | | |
| 2. Reproductive pathology gross measures- | | | | |
| hydrosaplinx or not and differences between the two | | | | |
| groups | | | | |
| 3. Antigen specific IgG and IgA antibody responses | | | | |
| in the vaginal lavage at 3 time points | | | | |

### Expected Positive Outcomes

The MuPharma device vaccination regimen shows significantly reduced shedding of chlamydia from the infected mice, when compared to the unvaccinated control, demonstrating the device elicited a protective mucosal immune response.
No difference or reduced size and reduced number of mice with visible hydrosalpinx in the MuPharma device vaccine group compared to control, indicating after vaccination with the MuPharma device the mice were not more likely (or even less likely) to have reproductive tract pathology in response to a chlamydial infection challenge.
In the test group, elevated (versus control group) presence of antibodies against the antigen in the reproductive secretions demonstrating that the MuPharma device vaccine delivery regimen elicits a B cell response specific to the antigens that can be detected at the reproductive mucosa.

### Example Nine: Evaluation of Intraocular Delivery of Avastin Using a Non-Invasive Ultrasonic Delivery Device

This project was designed to test whether MuPharma's proprietary ultrasonic delivery device delivers the Avastin antibody into the eye. Avastin is conventionally delivered to the eye for the treatment of retinal diseases by clinicians using an intra-vitreal injection into the vitreous fluid within the centre of the affected eye. There is a clear need for less invasive and safer methods of delivery that get therapeutic amounts of Avastin to the choroid and retina where retinal disease manifests.

### Aims:

1. To radiolabel and PET image Avastin administered to rabbit eyes.
2. To test whether MuPharma's proprietary ultrasonic delivery device improves Avastin antibody delivery into the eye.
3. To quantitate the amount of Avastin delivered at the eye.

### Methodology and Results:

Bioconjugation of NOTA-NCS to Avastin

Clinical Avastin Sample: 0.5 mg (25 mg mL⁻¹)

### Buffer: 80 μL 0.05 M HEPES, pH 8.6

NOTA-NCS (Macrocyclics, USA): 0.05 mg in 10 μL DMSO, 0.02 μmol (6 equivalents)
Reaction: room temperature for 1 h
Purification: 30 kDa centrifugal filter units (Amicon® Ultra, 500 μL, Merck Millipore, Billerica, Mass.).
Final volume: 25 μL
Final concentration: 20 mg ml
Labelling Reaction: 10 μl of NOTA-NCS Avastin with 1 ml of eluted Ga-68 for 30 minutes
Spin down 30 kDa centrifugal spin column

### Make to 25 μl

TABLE 14
Radiochemistry parameters

| | Rabbit 1 | | Rabbit 2 | | Rabbit 3 | |
|---|---|---|---|---|---|---|
| Eye | Left | Right | Left | Right | Left | Right |
| Ga- (MBq68 Activity) | 6.82 | 5.44 | 2.98 | 4.38 | 13.1 | 12.9 |
| Time | 10:09 | 10:08 | 13:12 | 13:12 | 16:06 | 16:04 |
| Avastin (μg) | 200 | 200 | 200 | 200 | 200 | 200 |
| Specific Activity (MBq/μg) | 0.0341 | 0.0272 | 0.0149 | 0.0219 | 0.0645 | 0.0655 |
| Cold Avastin (μg) | 0 | | 0 | | 0 | |
| Total Avastin (μg) | 200 | 200 | 200 | 200 | 200 | 200 |
| Total Volume (μl) | 25 | 25 | 25 | 25 | 25 | 25 |
| Volume in device (μl) | 7 | 7 | 7 | 7 | 7 | 7 |
| Total Avastin in device (μg) | 56 | 56 | 56 | 56 | 56 | 56 |

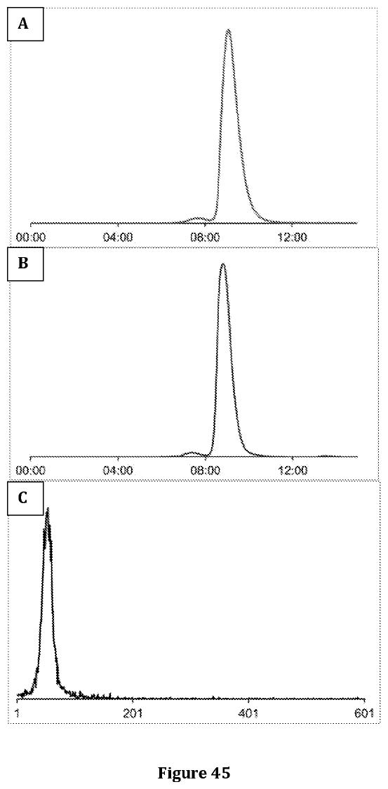

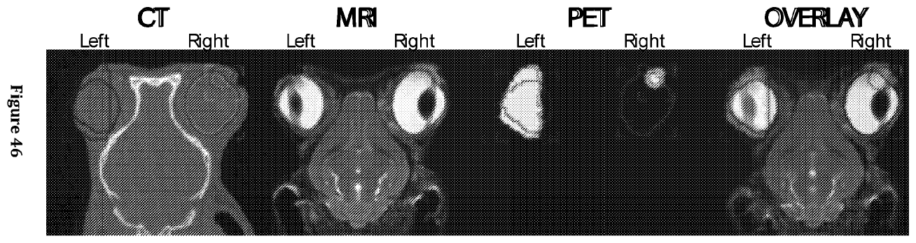

The radiopurity of the Avastin radiolabelling was checked with high performance liquid chromatography (HPLC). All traces show a single peak at the expected antibody elution time on an Agilent AvanceBio size-exclusion column. There is no evidence of antibody aggregation or free Ga-68, suggesting all the activity is associated with the antibody (FIG. 45). Avastin was eluted in buffer prepared with standard distilled water or standard saline depending upon requirements.

Ultrasound Delivery

The rabbits were anaesthetised with a mixture of medetomidine (0.2 mg/kg) and ketamine (15 mg/kg) by intramuscular injection. Anaesthesia was maintained using isoflurane between 1-3% in medical grade oxygen for the duration of the studies. All procedures relating to anaesthesia were carried out by a qualified Veterinary anaesthesiologist.

Radiolabelled Avastin solutions were loaded into each tip of the muPharma device as outlined in Table 15. For ultrasound test eyes, a constant power setting of 30 peak to peak voltage (Vpp) was used for the duration of the relevant eye. Control eyes involved no application of ultrasound to the relevant eye. The treatments applied to each eye are outlined in Table 15. The device was filled with Ga-68 radioactive Avastin for all studies and applied for the indicated time at the conjunctiva overlying sclera. Theoretically, a small amount of Avastin within the device may mix with tear film and consequently remain topically on the surface of the eye following application of the device. For all eyes following device application, a cautery device was used to mark a location approximately 3 mm away from the edge of the device tip application site and the edge of the eye was gently mopped with an absorbent sponge. The eye was closed with tape and the rabbit proceeded to the imaging bed. Other than for cautery burns, there were no visible damage, marks on or discoloration of the eye following administration. All radioactive material was initially counted and recovered post application, including the excess unloaded solution, the device tip and absorbent sponges and counted to determine residual and applied dose. The applied dose was obtained by decay correcting all values to a single time and then subtracting the initial radioactivity of Avastin in a vial where it was extracted and loaded into the device tip from the radioactivity remaining in the device tip following application to the eye and the radioactivity of sponges used to mop the eye following device application.

TABLE 15
Treatments applied to each eye.

| | Left Eye | | Right Eye | |
|---|---|---|---|---|
| | Treatment | Time (min) | Treatment | Time (min) |
| Rabbit 1 | | | | |
| Avastin | Ultrasound | 1:51 | Ultrasound | 4:30 |
| Rabbit 2 | | | | |
| Avastin | Control | 1:54 | Ultrasound | 3:00 |
| Rabbit 3 | | | | |
| Avastin | Ultrasound | 5:00 | Control | 5:00 |

Ultrasound indicates the transducer in the muPharma device was driven at 30 Vpp, Control indicates the device was filled and placed on the eye but no power was applied.

Imaging

Following device administration, each anesthetised animals was placed onto the positron emission tomography (PET) imaging bed and anesthesia was maintained with 1-3% isoflurane in 100% oxygen for the duration of the imaging session. The PET and computed tomography (CT) images were acquired on an Inveon PET-SPECT-CT instrument (Siemens). The head of the animal was positioned in the centre of the PET field of view and the animal was moved into the PET ring and list mode data was collected continuously for 60 minutes. The animal was moved and a co-localised CT image covering the PET field of view was acquired to generate PET attenuation correction maps. Following the CT scan the rabbits were moved to a 9.4 T magnetic resonance imaging (MRI) instrument (Hybrid Agilent and Bruker) and a 10 minute T2 RARE scan was completed on each animal's head using a Bruker 86 mm volume coil. The MRI parameters were; TR 4500 ms, TE 36 ms, RARE Factor 8 and 1 Average. The field of view was 80×64 mm with 48×1 mm slices and resolution of 0.25 mm. The CT data was reconstructed using a Feldkamp back projection algorithm and attenuation maps were generated using the Inveon acquisition workstation (IAW) software. The PET data was binned into 10 minute timeframes and reconstructed at 1 mm resolution, all radiation data was decay corrected to the PET start time or the time of measurement. To determine the relative amounts of Avastin a series of regions of interest (ROI) were drawn. Each eye was manually segmented from the overlaid MRI, PET and CT images as outlined in FIG. 2. The Inveon Research Workstation (IAW) software was used to co-register the CT, MRI and PET images and to analyse all the PET data. The presence of a positive PET signal was determined by thresholding signal twice the mean background of a region outside the rabbit. The eye was manually segmented from the MRI image as shown in FIG. 46 to ensure the activity was at the eye and not within any other region.

### Results

Radiation Results:

The radiation results following device application are outlined in Table 16. All were decay corrected to 55 minutes after the start of the PET imaging as this is the time the PET results were quantitated. The applied dose generally increases with the duration of the ultrasound, however, the applied dose for the 4:30 minute test eye was approximately comparable to the 1:54 minute control (no ultrasound) eye. Possible causes for the elevated control level are detailed in “Discussion of PET Quantitation” section in page 8. Table 3 outlines all the measured radiation parameters and related calculations including the maximum dose of radiolabelled Avastin potentially delivered by the device to a relevant eye through calculating the amount of radiation that is unaccounted for (applied dose). This was corrected for the 7 ul loaded into the tip of the device and then compared to a standard clinical preparation of Avastin. With the device active for 5 minutes there is the potential to deliver a dose of up to 9.61 ug of Avastin to the eye if a standard clinical formulation was loaded into the device and the applied dose represented the total administered dose. This also implies that the more concentrated clinical Avastin preparation would be delivered at the same rate as the radiolabelled more dilute Avastin samples used in this study.

TABLE 16
Radiation data at 55 minutes post PET imaging start time (Applied dose MBq).

| | Control | Control | US | US | US | US |
|---|---|---|---|---|---|---|
| | 2 min | 5 min | 2 min | 3 min | 4.5 min | 5 min |
| Activity | 1.0914 | 4.3751 | 1.5107 | 1.6042 | 1.9135 | 4.2209 |
| Residual | 0.9561 | 4.3577 | 1.4857 | 1.4489 | 1.7935 | 3.9893 |
| Applied Dose | 0.1353 | 0.0174 | 0.0250 | 0.1552 | 0.1199 | 0.2316 |
| Sp. Act. MBq/μg | 0.0054 | 0.0219 | 0.0096 | 0.008 | 0.0075 | 0.0211 |
| S. Act. Applied μg | 24.790 | 0.795 | 2.613 | 19.349 | 15.872 | 10.974 |
| Dose in tip μg | 200 | 200 | 200 | 200 | 200 | 200 |
| % Applied | 12.39 | 0.39 | 1.3 | 9.67 | 7.94 | 5.49 |
| Avastin μg in 7 μl | 175 | 175 | 175 | 175 | 175 | 175 |
| Max. dose μg | 19.39 | 0.668 | 2.275 | 16.92 | 13.88 | 9.61 |

All activities were decay corrected to 55 minutes post PET start time, the Activity is the initial activity of the vial for each application, residual is the measure of remaining activity after application, applied dose is the activity-residual, Sp. Act. is the specific activity following Ga68-Avastin labelling, S. Act. Applied is the applied dose divided by the specific activity in μg, dose in tip is the amount of labelled Avastin in the 7 μl loaded into the device tip, the % applied is the percentage of the applied dose from the 7 μl loaded into the tip, Avastin in 7 μl is the amount of Avastin contained in 7 μl of a standard intra-vitreal injection (conventional clinical Avastin delivery method), Max. dose is the % applied dose as a proportion of the amount of Avastin in a 7 μl clinical preparation.

PET Images and Quantitation:

### Time Post Administration:

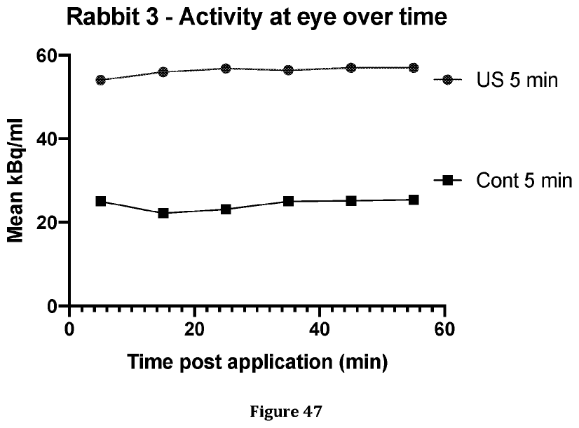

For each rabbit, there was a typical delay of between 10-20 minutes to transition from the device application bed to the PET imaging start-time due to the device application periods and the time required to stabilise the animals for PET imaging. All radioactive calculations are corrected to the time of sampling or the application time. To determine if there was a change in the amount of Avastin post-application over the 60 minute imaging duration, the data was binned into 10 minute frames. The decay-corrected representative results from rabbit 3 are shown in FIG. 47 below, there was very little change in the amount of Avastin at the eye during the imaging time course post-administration. It is unknown whether the distribution of Avastin changes immediately post-application (due to ocular clearance mechanisms), but it is clear it remains static for the period of 15-80 minutes post application. This stable distribution was observed for all rabbit eyes studied.

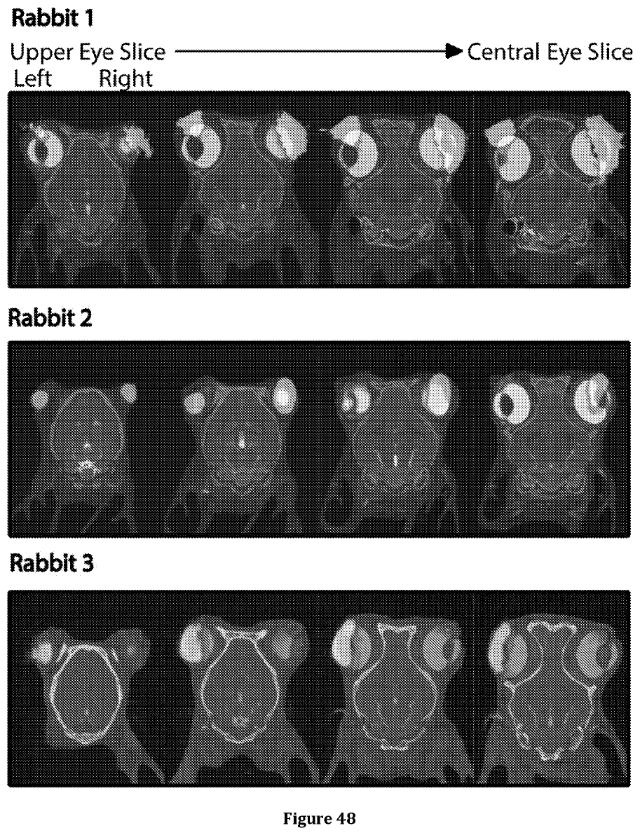

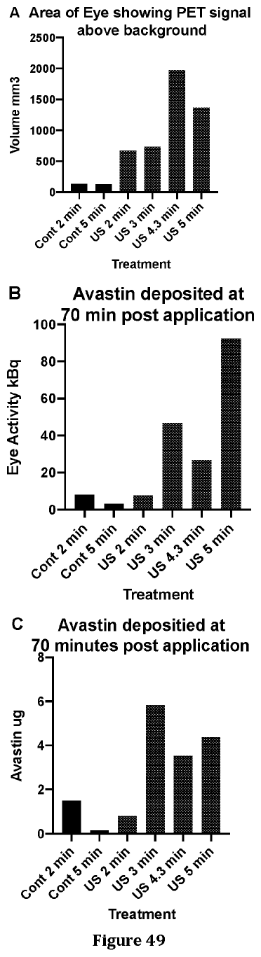

As the signal above or within the eye showed little deviation, the following images and quantitation used the 50-60 minute timeframe post PET acquisition (approximately 70-85 minutes post application) to show the maximal distribution of Avastin over time. The imaging clearly showed a large region of positive signal at the top of each eye indicating a portion of Avastin remains at the application site (FIGS. 47 and 48). The lower portion of the eye was not positive, only the region of application with a limited range of diffusion over or through the eye. There was minimal evidence of Avastin within the drainage channels of the eye (FIG. 48). Activating the ultrasound clearly deposits a larger quantity of radiation onto the eye at the application site and the longer the device is active also increases deposition (FIGS. 48 and 49). There was little evidence in the PET signal of activity that was not at the deposition site, there was no significant activity at the rear of the eye, although it should be mentioned that it is unknown if the Avastin antibody binds the rabbit vascular endothelial growth factor (VEGF) protein or whether this would even be expressed in the healthy rabbits used in the study.

The results in Table 16 indicated a theoretical maximal amount of Avastin that the ultrasonic device could apply to the eye under the different conditions tested. This was based on the missing activity as measured by the difference between the decay corrected initial and residual radiation measures. In Table 17, the PET data as mean MBq/ml was multiplied by the size of each region of interest on the eye in mm³ to obtain an activity value for the total activity remaining at the eye 55 minutes after the beginning of the PET acquisition. This value was corrected with the specific activity to calculate the μg at the eye for each treatment. The PET value was divided by the Avastin amount loaded into the device (56 μg, see Table 14) to calculate the % loaded dose. This was used to generate a theoretical maximal amount of Avastin that would be applied if a clinical Avastin sample (175 μg in 7 μl) was loaded into the tip of the device. It appears that applying the device for 5 minutes can deposit up to 13.66 μg of Avastin assuming that the more concentrated clinical Avastin sample is delivered at the same rate as the more dilute radiolabelled sample used in this study.

TABLE 17
Avastin measures from quantitation of PET activity.

| | Control | Control | US | US | US | US |
|---|---|---|---|---|---|---|
| | 2 min | 5 min | 2 min | 3 min | 4.3 min | 5 min |
| PET MBθ | 0.00813 | 0.00332 | 0.007685 | 0.0468 | 0.02664 | 0.09229 |
| S. Act. MBq/μg | 0.0054 | 0.0219 | 0.0096 | 0.008 | 0.0075 | 0.0211 |
| Avastin PET μg | 1.4899 | 0.1515 | 0.8033 | 5.8389 | 3.5267 | 4.3731 |
| Avastin in 7 μl (μg) | 56 | 56 | 56 | 56 | 56 | 56 |
| % loaded dose | 2.6605 | 0.2709 | 1.4344 | 10.426 | 6.2976 | 7.809 |
| Avastin μg in 7 μl | 175 | 175 | 175 | 175 | 175 | 175 |
| Max. dose μg | 4.65 | 0.48 | 2.51 | 18.24 | 11.02 | 13.66 |

The PET eye measure is the estimated activity (MBq) in each eye ROI, S. Act. Is the specific activity of each preparation, Avastin PET μg is the PET MBq/specific activity, the Avastin in 7 μl is the amount of Avastin in 7 μl of a clinical preparation of Avastin, and the Max. Dose is the maximum theoretical dose if a clinical sample of Avastin was loaded into the device.

### Discussion of PET Quantitation

Accurate quantification of the activity entering the eye at the site of application is difficult as the clearance kinetics could not be determined in this study. Standard PET methods such as standardised uptake values and other measures are meaningless as there is no even distribution within the animal. To fully demonstrate clearance kinetics the time from application to imaging will need to be shortened, standards will need to be run with the animal and blood measurements taken. There could be rapid clearance (particularly with the choroid) of free Avastin penetrating the eye tissues during and immediately after application. Due to the 1 mm resolution limit of PET imaging, it cannot determined in what tissues Avastin was detected including at the device application site, however, the stable distribution over time may indicate that detection is associated with tissue structures that are hampering Avastin diffusion throughout other tissues which theoretically could be due to local drug penetration. Also, it is a possibility that some topical radiolabelled Avastin from the device remaining after the application entered into the tissue through the cautery burn sites. It is clear that the majority of Avastin remained on the ocular surface or within the eye tissues, this may have been due to the rabbit being asleep with the eyes closed, thus minimising mechanical clearance by normal blinking.

### CONCLUSIONS

The resolution of the PET was able to differentiate the application site within the eye and other cranial structures indicating it is a suitable methodology to determine Avastin biodistribution over time within the head and eyes. There is clear evidence that muPharma device ultrasound mediated delivery enhances the deposition of Avastin on to the eye compared to muPharma device non-ultrasonic delivery. Increasing the duration of ultrasound delivery increases the amount of Avastin deposited at the eye. The deposited Avastin antibody remains at the site of application for at least 80 minutes post-delivery with little loss of drug and only minimal diffusion. The maximal achievable dose of a clinical preparation was quantitated by determining the applied dose to try to estimate how much the device can deliver. The Avastin signal within the eye was independently quantitated using the radioactive data and the PET data from the positive regions of interest and calibration of the scanner with Gallium-68. Correcting both of these measures for the known specific activity indicates that the maximal delivered dose is in the low microgram range and is significantly increased with the addition of ultrasound. The typical clinical preparation of Avastin is much more concentrated that that used in this study and correcting for this factor indicates the device can potentially deliver up to 13.66 μg of Avastin to the eye after a 5 minute application time (Tables 16 and 17).

1. A device, comprising:
an agent carrier comprising an agent transfer surface for non-invasive delivery of an agent into a tissue, and a plurality of nanoscale channels extending partially or wholly through the agent carrier to the agent transfer surface enabling retention of the agent and/or transportation of the agent to the tissue, wherein the agent carrier comprises or is acoustically couplable to a piezoelectric substrate;
an electrode electrically couplable to the piezoelectric substrate; and
a controller electrically couplable to the electrode and configured to apply an electrical signal to the electrode to propagate an acoustic wave on and/or in the piezoelectric substrate which is capable of delivering the agent from the agent carrier into the tissue.

2. The device of claim 1, wherein at least: 50%, 60%, 70%, 80%, 90%, 95%; or all of the plurality of nanoscale channels, have a maximum width exceeding the maximum width of the agent by no more than: 1.2 fold (1.2×), 1.5-fold (×1.5), two-fold (×2), three-fold (×3), four-fold (×4), five-fold (×5), ten-fold (×10), twenty-fold (×20), thirty-fold (×30), forty-fold (×40), or fifty-fold (×50).

3. The device of claim 1 or claim 2, wherein at least: 50%, 60%, 70%, 80%, 90%, 95%; or all of the plurality of nanoscale channels, have a maximum width exceeding the maximum width of the agent by no more than: 1%, 2%, 3%, 4%, 5%, 10%, or 20%.

4. The device of any one of claims 1 to 3, wherein:
the plurality of nanoscale channels terminate as pores at the agent transfer surface; and the pores have a maximum width exceeding the maximum width of the agent by no more than: 1.2 fold (1.2×), 1.5-fold (×1.5), two-fold (×2), three-fold (×3), four-fold (×4), five-fold (×5), ten-fold (×10), twenty-fold (×20), thirty-fold (×30), forty-fold (×40), or fifty-fold (×50).

5. The device of any one of claims 1 to 4, wherein:
the plurality of nanoscale channels terminate as pores at the agent transfer surface; and
the pores have a maximum width exceeding the maximum width of the agent by no more than: 1%, 2%, 3%, 4%, 5%, 10%, or 20%.

6. The device of any one of claims 1 to 5, wherein at least: 50%, 60%, 70%, 80%, 90%, 95%; or all of the plurality of nanoscale channels, have a maximum width of below: 65 nm, 55 nm, 50 nm, 24 nm or 10 nm; or a maximum width of between 160 nm and 999 nm, 160 nm and 300 nm, 160 nm and 450 nm, 160 nm and 600 nm, 160 nm and 750 nm, 160 nm and 900 nm, or 160 nm and 999 nm.

7. The device of any one of claims 1 to 6, wherein the plurality of nanoscale channels range in maximum width (e.g. diameter) from between 1 nm and 55 nm, from between 1 nm and 50 nm, from between 1 nm and 45 nm, from between 1 nm and 40 nm, from between 1 nm and 35 nm, from between 5 nm and 55 nm, from between 5 nm and 50 nm, from between 5 nm and 45 nm, from between 5 nm and 40 nm, from between 5 nm and 35 nm, from between 10 nm and 55 nm, from between 10 nm and 50 nm, from between 10 nm and 45 nm, from between 10 nm and 40 nm, and from between 10 nm and 35 nm.

8. The device of any one of claims 1 to 7, wherein the porosity of the plurality of nanoscale channels accounts for up to 60%, 70%, 80% or 85% of the total agent carrier volume.

9. The device of any one of claims 1 to 8, wherein:
the plurality of nanoscale channels extend from the interior of the agent carrier body and terminate as pores at the agent transfer surface.

10. The device of any one of claims 1 to 9, wherein at least 80%, at least 90%, at least 95% or all of the pores have a maximum width below: 65 nm, 55 nm, 50 nm, 24 nm or 10 nm.

11. The device of any one of claims 1 to 10, wherein the plurality of nanoscale channels are provided in an amount of at least: 5000, 6000, 7000, 8000, 9000, 10000, 11000, 12000, 13000, 14000, 15000, 20000, 25000, 50000, 75000 or 100000 channels/cm² of the agent carrier body.

12. The device of any one of claims 1 to 11, wherein the plurality of nanoscale channels is fabricated using any one or more of: silicon, porated silicon, germanium, graphene, synthetic polymer or a combination thereof.

13. The device of any one of claims 1 to 11, wherein the plurality of nanoscale channels is fabricated using three-dimensional (3D) printing of a material selected from the group consisting of: polymeric material, metallic material, ceramic material, and any combination thereof.

14. The device of any one of claims 1 to 13, further comprising an acoustic generator capable of generating a secondary acoustic excitation frequency capable of modulating a primary acoustic excitation frequency generated by the piezoelectric substrate, wherein the secondary acoustic excitation frequency is less than or equal to the primary acoustic excitation frequency.

15. The device of any one of claims 1 to 14, wherein the device:
does not comprise an electrode for contacting the tissue surface, and/or
is not configured to utilise repulsive electromotive force to transport a charged agent into and/or through the tissue in contact with the agent transfer surface.

16. The device of any one of claims 1 to 15, wherein:
the agent carrier comprises the piezoelectric substrate,
the piezoelectric substrate comprises the agent transfer surface, and
the agent is present on the agent transfer surface.

17. The device of claim 16, wherein the agent is functionalised and/or lyophilised on the agent transfer surface.

18. The device of any one of claims 1 to 17, wherein the device is non-invasive, and the agent transfer surface does not comprise microneedles.

19. A method for delivering an agent to an internal layer within a target tissue, the method comprising:
contacting the target tissue with the agent transfer surface of the device of any one of claims 1 to 18, and
applying an electrical signal to the electrode of the device to propagate acoustic waves on and/or in the piezoelectric substrate of the device, and thereby deliver the agent from the agent transfer surface to the internal layer of the target tissue.

20. The method of claim 19, wherein the method comprises delivering the agent into or through any one or more of: epithelium, sub-epithelium, mucosa, sub-mucosa, mucous membrane vasculature, nasal septum, cornea, corneal epithelium, Bowman's membrane, corneal stroma, corneal endothelium, conjunctiva, Tenon's fascia, episclera, sclera, choroid, choriocapillaris, Bruch's membrane, retinal pigment epithelium, neural retina, retinal blood vessels, internal limiting membrane, vitreous humour, a component of the gastro-intestinal system, a component of the genito-urinary, a component of the reproductive system (e.g. vagina, uterus, testes), a component of the respiratory system, a component of the ocular system, a component of the auditory system, an eye, an ear, and a lip.

21. The method of claim 19 or claim 20, wherein:
the target tissue is intact tissue, and
the agent transfer surface is configured to inhibit or prevent mechanical penetration of a surface of the target tissue and to prevent piercing or destruction of the tissue by ultrasonic waves emanating from the device, when in contact with the tissue during standard use of the device.

22. The method of any one of claims 19 to 21, wherein the target tissue is mucosal tissue, or the eye.

23. The method of claim 22, wherein the mucosal tissue is intact, the agent transfer surface does not penetrate an intact epithelial layer of the mucosal tissue during standard use of the device, and wherein delivery of a therapeutically effective amount of the agent into the mucosal tissue induces an immune response in the subject.

24. The method of claim 23, wherein the immune response is at least a mucosal immune response.

25. The method of claim 24, wherein the mucosal immune response is induced by controlling the amount of agent delivered into an epithelial layer of the mucosal tissue, or into the epithelial and sub-epithelial layers of a mucous membrane.

26. The method of claim 23, wherein the immune response is a systemic immune response.

27. The method of claim 26, wherein delivery of the agent to induce a systemic immune response is by controlling the amount of agent delivered into and through the epithelial and sub-epithelial tissue.

28. The method of claim 22, wherein the target tissue is the eye, and the method comprises contacting the agent transfer surface with corneal epithelium and delivering a target amount of the agent into the cornea of the eye.

29. The method of claim 28, wherein:
the agent is delivered for the treatment of myopia or keratoconus,
the agent is a therapeutically effective amount of any one or more of riboflavin-5-phosphate sodium salt, glutaraldehyde, grape seed extract, and/or genipin, and
the method further comprises exposing the cornea to ultraviolet light following delivery of the therapeutic amount of the agent to the cornea for a time period sufficient to induce collagen crosslinking in the cornea.

30. The method of claim 29, further comprising repeating the delivery of the therapeutically effective amount and the exposure to ultraviolet light within 1, 2, 3, 4, 5, 6, 7, 14, 21, 28, 42 or 60 days.

31. The method of claim 28, wherein:
the agent comprises a therapeutic amount of the agent for treating a condition or disease upon delivery to the posterior segment of the eye,
and the therapeutically effective amount of the agent
is delivered through the corneal epithelium, Bowman's membrane, Corneal stroma, Descemet's membrane and Corneal endothelium, into aqueous humor,
circulates within the aqueous humor through the pupil and around the lens into the posterior chamber,
contacts one or more of: vitreous humor, ciliary body blood vessels, uveal blood vessels in the pars plana, and
is distributed via the choroidal vasculature to the posterior segment of the eye.

32. The method of claim 22, wherein:
the agent comprises a therapeutic amount of the agent for treating a condition or disease upon delivery to the posterior segment of the eye,
and the therapeutically effective amount of the agent
is delivered through the conjunctiva overlying the sclera, and the sclera,
enters the uveal tract of the eye,
is distributed via the choroidal vasculature to the choroid and retina in the posterior segment of the eye.

33. The method of claim 31 or claim 32, wherein the therapeutically effective amount of the agent comprises anti-Vascular Endothelial Growth Factor (anti-VEGF) agents, nucleic acids, and/or an anti-inflammatory drug, and is delivered for the treatment of Age Related Macular Degeneration, Diabetic Eye Disease, or Posterior Choroiditis.

34. The method of any one of claims 19 to 33, wherein propagating the acoustic wave comprises generating ultrasonic power in the range 0.05 to 5.25 Wcm⁻², or 0.05 to 0.7 Wcm⁻², for the delivery of the agents into the target tissue.

35. The method of any one of claims 19 to 34, comprising generating a primary acoustic excitation frequency on and/or in the piezoelectric substrate of less than 1 mHz, between 35 kHz and 50 kHZ, 35 kHZ and 55 kHZ, or above 1 mHz.

36. The method of claim 35, further comprising generating one or more secondary acoustic excitation frequencies on and/or in the piezoelectric substrate to thereby modulate the primary acoustic excitation on and/or in the piezoelectric substrate.

37. The method of claim 36, wherein the secondary acoustic excitation frequency is less than or equal to the primary acoustic excitation frequency.

38. The device of any one of claims 1 to 18, wherein the delivering comprises transportation of the agent through the nanoscale channels by the acoustic waves to the agent transfer surface.

39. The device of any one of claim 1 to 18 or 38, wherein the delivering comprises continuous operation of the device over a time period of more than: one minute, two minutes, three minutes, four minutes, 5 minutes or 10 minutes.

40. The method of any one of claims 19 to 37, wherein the delivering comprises transportation of the agent through the nanoscale channels by the acoustic waves to the agent transfer surface.

41. The method of any one of claims to 19 to 37 or 40, wherein the delivering comprises continuous operation of the device over a time period of more than: one minute, two minutes, three minutes, four minutes, 5 minutes or 10 minutes.

42. The device of any one of claim 1 to 18, 38 or 39, wherein the device comprises an internal reservoir in fluid communication with the nanoscale channels and comprising some or all of the agent.

43. The method of any one of claim 19 to 37, 40 or 41, wherein the device comprises an internal reservoir in fluid communication with the nanoscale channels and comprising some or all of the agent.
